SEQUENCE LISTING (159 sequences)

SEQ ID NO: 1 - protein, 481 aa, synthetic construct
  REGION 1..481: note = amino acid sequence of human full-length CD33 (MDT000208; hsCD33-mmFc; Immunogen for human full-length)
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGGGSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 2 - protein, 244 aa, synthetic construct
  REGION 1..244: note = amino acid sequence of human DeltaE2 version of CD33 with a mouse Fc domain
MPLLLLLPLLWADLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGGGSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTIS

SEQ ID NO: 3 - protein, 480 aa, synthetic construct
  REGION 1..480: note = amino acid sequence of Macaca fascicularis CD33 full-length with a mouse Fc domain
MPLLLLPLLWAGALAMDPRVRLEVQESVTVQEGLCVLVPCTFFHPVPYHTRNSPVHGYWFREGAIVSLDSPVATNKLDQEVQEETQGRFRLLGDPSRNNCSLSIVDARRRDNGSYFFRMEKGSTKYSYKSTQLSVHVTDLTHRPQILIPGALDPDHSKNLTCSVPWACEQGTPPIFSWMSAAPTSLGLRTTHSSVLIITPRPQDHGTNLTCQVKFPGAGVTTERTIQLNVSYASQNPRTDIFLGDGSGRKARKQGGGSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 4 - protein, 489 aa, synthetic construct
  REGION 1..489: note = amino acid sequence of the extracellular domain of mouse CD33 full length where the C2-set Ig-like domain from mouse has been replaced with the C2-set Ig-like domain from human CD33, combined with a with a Fc region from human IgG1
MLWPLPLFLLCAGSLAQDLEFQLVAPESVTVEEGLCVHVPCSVFYPSIKLTLGPVTGSWLRKGVSLHEDSPVATSDPRQLVQKATQGRFQLLGDPQKHDCSLFIRDAQKNDTGMYFFRVVREPFVRYSYKKSQLSLHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 280 aa, synthetic construct
  REGION 1..280: note = amino acid sequence of the extracellular domain of CD33 full-length with a 6-histidine-avidin tag
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGSHHHHHHGSGLNDIFEAQKIEWHE

SEQ ID NO: 6 - protein, 279 aa, synthetic construct
  REGION 1..279: note = amino acid sequence of the extracellular domain of Macaque fascicularis CD33 full-length with a 6-histidine-avidin tag
MPLLLLPLLWAGALAMDPRVRLEVQESVTVQEGLCVLVPCTFFHPVPYHTRNSPVHGYWFREGAIVSLDSPVATNKLDQEVQEETQGRFRLLGDPSRNNCSLSIVDARRRDNGSYFFRMEKGSTKYSYKSTQLSVHVTDLTHRPQILIPGALDPDHSKNLTCSVPWACEQGTPPIFSWMSAAPTSLGLRTTHSSVLIITPRPQDHGTNLTCQVKFPGAGVTTERTIQLNVSYASQNPRTDIFLGDGSGRKARKQGSHHHHHHGSGLNDIFEAQKIEWHE

SEQ ID NO: 7 - protein, 153 aa, synthetic construct
  REGION 1..153: note = amino acid sequence of the extracellular domain of CD33DeltaE2 with a 6-histidine-avidin tag
MPLLLLLPLLWADLTHRPKILIPGTLEPGHSKNLTCSVSWASEQGTPPIFSWLSAAPTSLGPRTTHSSVLIITPRPQDHGTNLTCQVKFAGAGVTTERTIQLNVTYVPQNPTTGIFPGDGSGKQETRAGSHHHHHHGSGLNDIFEAQKIEWHE

SEQ ID NO: 8 - protein, 234 aa, synthetic construct
  REGION 1..234: note = amino acid sequence of the light chain of IgG2a anti-CD33 antibody 5D12
METDTLLLWVLLLWVPGSTGDIKMTQSPSSIYASLGERVTINCKASQDIKSYLSWYQQKPWKSPKTLIYYATTLADGVPSRFSGSGSGQDYSLTISSLESDDTATYYCLHHGESPWTFGEGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 471 aa, synthetic construct
  REGION 1..471: note = amino acid sequence of the heavy chain of IgG2a anti-CD33 antibody 5D12
METDTLLLWVLLLWVPGSTGQVQLQQSGAEVVKPGASVKISCRASGYAFSNYWMNWVKQRPGKGLEWIGQIYPGNFNTDYNGQFKGKATLTVDKSSNTAYMQLSSLTSEDSAVYFCARFFDFGAYFTLDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 240 aa, synthetic construct
  REGION 1..240: note = amino acid sequence of the light chain of IgG2a anti-CD33 antibody 8F5
METDTLLLWVLLLWVPGSTGDIVMSQSPSSLPVSVGEKVTLSCKSSQSLLYSRNQYNFLAWYQQRPGQSPKLLIYWASTRESGVPDRFTGSGSGTDFTLTISSVKAEDLAVYYCQQYYSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 469 aa, synthetic construct
  REGION 1..469: note = amino acid sequence of the heavy chain of IgG2a anti-CD33 antibody 8F5
METDTLLLWVLLLWVPGSTGEVKLVESGGGLVQPGGSLKLSCAASGFTFSDFYMYWVRQTPEKRLEWVAFISNAGVTTYYPDTVEGRFTISRDNAKNTLYLQMSRLMSEDTAMYYCTKSDYDGAWFPYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 12 - protein, 239 aa, synthetic construct
  REGION 1..239: note = amino acid sequence of the light chain of IgG2b anti-CD33 antibody 12B12
METDTLLLWVLLLWVPGSTGDIVMTQAAFSNPVTLGTSASISCRSSQSLLHSNGITYLYWYLQKPGQSPQLLIYQMSNLASGVPDRFSSSGSGTDFTLRISRVEAEDVGVYYCAQNLELPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - protein, 473 aa, synthetic construct
  REGION 1..473: note = amino acid sequence of the heavy chain of IgG2b anti-CD33 antibody 12B12
METDTLLLWVLLLWVPGSTGEVQLQQSGTVLARPGASVKMSCKASGYTFTTYWMHWIKQSPGQGLEWIGAIYPGNSDTSYNQKFKGKAKLTAVTSASTAYMELSSLTNEDSAVYYCEIYDGYHFIYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 14 - protein, 238 aa, synthetic construct
  REGION 1..238: note = amino acid sequence of the light chain of IgG1 anti-CD33 antibody 4H10
METDTLLLWVLLLWVPGSTGDVVMTQTPLTLSVTIGQPASISCKSSQSLLYSNGKTYLHWFLQRPGQSPKRLIYLVSKLDSGVPDRFTGSGSGTDFTLKISRVEAEDLGVYYCVQGTHFPRTFGGGTKLEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 15 - protein, 464 aa, synthetic construct
  REGION 1..464: note = amino acid sequence of the heavy chain of IgG1 anti-CD33 antibody 4H10
METDTLLLWVLLLWVPGSTGQVQLQQSGAELVRPGTSVKVSCKASGYAFTNYLIEWVKQRPGQGLEWIGVIHPGNNSTSYNAKFRGKATLTADRSSSTAYMQLSSLTSEDSAVYFCARYGYDERNAMDYWGQGTSVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 16 - protein, 239 aa, synthetic construct
  REGION 1..239: note = amino acid sequence of the light chain of IgG1 anti-CD33 antibody 11D5
METDTLLLWVLLLWVPGSTGDIVMTQAAFSNPVTLGTSASISCRSNKSLLHSNGITYLYWYLQKPGQSPQLLIYQMSNLASGVPDRFSSSGSGTDFTLRISRVEAEDVGVYYCAQNLELPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - protein, 461 aa, synthetic construct
  REGION 1..461: note = amino acid sequence of the heavy chain of IgG1 anti-CD33 antibody 11D5
METDTLLLWVLLLWVPGSTGEVQFQQSETVLARPGTSVKLSCKASGYTFTSYWMHWLKQRPGQGLEWIGAIYCGNSDTSYNQKFKGKAKLTAVTSATTAYMELSSLTNEDSAVYYCKIYDGYHFDYWGQGTTLTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 18 - protein, 227 aa, synthetic construct
  REGION 1..227: note = amino acid sequence of the light chain of IgG1 anti-CD33 antibody 13E11
METDTLLLWVLLLWVPGSTGDIVLTQSPVSLAVSLGQRATISCKASHGVEYAGAHYMNWYQQKPGQPPKLLIYAASNLGSGTDFTLNIHPVEEEDSATYYCQQSNEDPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 460 aa, synthetic construct
  REGION 1..460: note = amino acid sequence of the heavy chain of IgG1 anti-CD33 antibody 13E11
METDTLLLWVLLLWVPGSTGKVQLQQSGAELVKPGASVKLSCKASGYTFTDYTLHWLKQRSGQGLEWIGWFYPTSGSINYNERFKDKATLTADKSSSTVYMELSRLTSVDSAVYFCARHKFGFDYWGQGTTLTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 20 - protein, 234 aa, synthetic construct
  REGION 1..234: note = amino acid sequence of the light chain of IgG1 anti-CD33 antibody 5E10.7
METDTLLLWVLLLWVPGSTGDIKMTQSPSSIYASLGERVTITCKASQDIKSYLSWYQQKPRKSPKTLIYYATTLADGVPSRFSGSGSGQDFSLTISSLESDDTATYYCLHHSESPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 462 aa, synthetic construct
  REGION 1..462: note = amino acid sequence of the heavy chain of IgG1 anti-CD33 antibody 5E10.7
METDTLLLWVLLLWVPGSTGQVQLQQPGAEFVKPGASVKLSCKASGYTFTSYWMQWVKQRPGQGLEWIGEIDSSDSYTNYNQKFKGMATLTVDRSSSTAYMQLSSLTSEDSAVYYCARSRPYDWFPYWGQGTLVTVSTAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 22 - protein, 234 aa, synthetic construct
  REGION 1..234: note = amino acid sequence of the light chain of IgG1 anti-CD33 antibody 11D11
METDTLLLWVLLLWVPGSTGDIQMTQTTSSLSASLGDRVTISCRASQDISNYLNWYQQKPDGTVKLLIYYTSRLHSGVPSRFSGSGSGTDYSLTISNLEQEDIATYFCQQGSTLPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 23 - protein, 462 aa, synthetic construct
  REGION 1..462: note = amino acid sequence of the heavy chain of IgG1 anti-CD33 antibody 11D11
METDTLLLWVLLLWVPGSTGEVNLVESGGGLVQSGRSLRLSCATSGFTFSDFYMEWVRQAPGKGLEWIAASRNKANDYTTEYKASVKGRFIVSRDTSQSILYLQMNALRAEDTAIYYCTRDTGPMDYWGQGTSVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 24 - protein, 239 aa, synthetic construct
  REGION 1..239: note = amino acid sequence of the light chain of IgG1 anti-CD33 antibody 7E7
METDTLLLWVLLLWVPGSTGDVVMTQTPLILSVTIGQPASISCKSSQSLLDSDGKTYLSWLLQRPGQSPKRLIHLVSKLDSGVPDRFTGSGSGTDFTLKISRVEAEDLGVYYCWQGTHFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - protein, 463 aa, synthetic construct
  REGION 1..463: note = amino acid sequence of the heavy chain of IgG1 anti-CD33 antibody 7E7
METDTLLLWVLLLWVPGSTGQVTLKESGPGILQPSQTLSLTCSFSGFSLNSYGMGIGWIRQPSGKGLEWLAHIWWDDNKYYKPDLKSRLTVSKDTSKNQVFLKIANVDTTDTATYFCARDGGYSLFAYWGQGTLVTVSVAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 26 - protein, 117 aa, synthetic construct
  REGION 1..117: note = amino acid sequence of human V-set Ig-like domain
PNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLS

SEQ ID NO: 27 - protein, 84 aa, synthetic construct
  REGION 1..84: note = amino acid sequence of human C2-set Ig-like domain
PKILIPGTLEPGHSKNLTCSVSWACEQGTPPIFSWLSAAPTSLGPRTTHSSVLIITPRPQDHGTNLTCQVKFAGAGVTTERTIQ

SEQ ID NO: 28 - protein, 19 aa, synthetic construct
  REGION 1..19: note = amino terminal peptide (neoepitope) in CD33 confirmed by mass spectrophotometry (MS)
MPLLLLLPLLWADLTHRPK

SEQ ID NO: 29 - protein, 19 aa, synthetic construct
  REGION 1..19: note = amino terminal peptide (neoepitope) in CD33 confirmed by mass spectrophotometry (MS)
  MOD_RES 1: note = Met is oxidized
MPLLLLLPLLWADLTHRPK

SEQ ID NO: 30 - protein, 17 aa, synthetic construct
  REGION 1..17: note = amino terminal peptide (neoepitope) in CD33 confirmed by mass spectrophotometry (MS)
LLLLLPLLWADLTHRPK

SEQ ID NO: 31 - protein, 18 aa, synthetic construct
  REGION 1..18: note = amino terminal peptide (neoepitope) in CD33 confirmed by mass spectrophotometry (MS)
PLLLLLPLLWADLTHRPK

SEQ ID NO: 32 - protein, 364 aa, synthetic construct
  REGION 1..364: note = amino acid sequence of human full-length CD33
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 33 - protein, 237 aa, synthetic construct
  REGION 1..237: note = amino acid sequence of the DeltaE2 version of CD33
MPLLLLLPLLWADLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 34 - protein, 310 aa, synthetic construct
  REGION 1..310: note = amino acid sequence of CD33E7a which lacks most of the intracellular domain of human CD33 (C-terminal truncation)
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPVR

SEQ ID NO: 35 - protein, 282 aa, synthetic construct
  REGION 1..282: note = amino acid sequence of cynomolgus CD33
MPLLLLPLLWAGALAMDPRVRLEVQESVTVQEGLCVLVPCTFFHPVPYHTRNSPVHGYWFREGAIVSLDSPVATNKLDQEVQEETQGRFRLLGDPSRNNCSLSIVDARRRDNGSYFFRMEKGSTKYSYKSTQLSVHVTDLTHRPQILIPGALDPDHSKNLTCSVPWACEQGTPPIFSWMSAAPTSLGLRTTHSSVLIITPRPQDHGTNLTCQVKFPGAGVTTERTIQLNVSYASQNPRTDIFLGDGSGRKARKQGVVQGAIGGAGVTVLLALCLCLIFFTVQ

SEQ ID NO: 36 - protein, 283 aa, synthetic construct
  REGION 1..283: note = amino acid sequence of human CD33 (HsCD33_P20138) shown in the alignment in FIG. 2
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVQ

SEQ ID NO: 37 - protein, 267 aa, synthetic construct
  REGION 1..267: note = amino acid sequence of murine CD33 (MmCD33_Q63994) shown in the alignment in FIG. 2
MLWPLPLFLLCAGSLAQDLEFQLVAPESVTVEEGLCVHVPCSVFYPSIKLTLGPVTGSWLRKGVSLHEDSPVATSDPRQLVQKATQGRFQLLGDPQKHDCSLFIRDAQKNDTGMYFFRVVREPFVRYSYKKSQLSLHVTSLSRTPDIIIPGTLEAGYPSNLTCSVPWACEQGTPPTFSWMSTALTSLSSRTTDSSVLTFTPQPQDHGTKLTCLVTFSGAGVTVERTIQLNVTRKSGQMRELVLVAVGEATVKLLILGLCLVFLIVMF

SEQ ID NO: 38 - protein, 10 aa, synthetic construct
  REGION 1..10: note = CDRL1 of anti-CD3 antibody OKT3
SASSSVSYMN

SEQ ID NO: 39 - protein, 11 aa, synthetic construct
  REGION 1..11: note = CDRL2 of anti-CD3 antibody OKT3
RWIYDTSKLAS

SEQ ID NO: 40 - protein, 9 aa, synthetic construct
  REGION 1..9: note = CDRL3 of anti-CD3 antibody OKT3
QQWSSNPFT

SEQ ID NO: 41 - protein, 13 aa, synthetic construct
  REGION 1..13: note = CDRH1 of anti-CD3 antibody OKT3
KASGYTFTRYTMH

SEQ ID NO: 42 - protein, 16 aa, synthetic construct
  REGION 1..16: note = CDRH2 of anti-CD3 antibody OKT3
INPSRGYTNYNQKFKD

SEQ ID NO: 43 - protein, 10 aa, synthetic construct
  REGION 1..10: note = CDRH3 of anti-CD3 antibody OKT3
YYDDHYCLDY

SEQ ID NO: 44 - protein, 242 aa, synthetic construct
  REGION 1..242: note = amino acid sequence of a scFv derived from OKT3 which retains the capacity to bind CD3
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 45 - protein, 11 aa, synthetic construct
  REGION 1..11: note = CDRL1 of anti-CD3 antibody 20G6-F3
QSLVHNNGNTY

SEQ ID NO: 46 - skipped

SEQ ID NO: 47 - protein, 9 aa, synthetic construct
  REGION 1..9: note = CDRL3 of anti-CD3 antibody 20G6-F3
GQGTQYPFT

SEQ ID NO: 48 - protein, 8 aa, synthetic construct
  REGION 1..8: note = CDRH1 of anti-CD3 antibody 20G6-F3
GFTFTKAW

SEQ ID NO: 49 - protein, 10 aa, synthetic construct
  REGION 1..10: note = CDRH2 of anti-CD3 antibody 20G6-F3
IKDKSNSYAT

SEQ ID NO: 50 - protein, 12 aa, synthetic construct
  REGION 1..12: note = CDRH3 of anti-CD3 antibody 20G6-F3
RGVYYALSPFDY

SEQ ID NO: 51 - protein, 11 aa, synthetic construct
  REGION 1..11: note = CDRL1 of anti-CD3 antibody 4B4-D7
QSLVHDNGNTY

SEQ ID NO: 52 - skipped

SEQ ID NO: 53 - protein, 9 aa, synthetic construct
  REGION 1..9: note = CDRL3 of anti-CD3 antibody 4B4-D7
GQGTQYPFT

SEQ ID NO: 54 - protein, 8 aa, synthetic construct
  REGION 1..8: note = CDRH1 of anti-CD3 antibody 4B4-D7
GFTFSNAW

SEQ ID NO: 55 - protein, 10 aa, synthetic construct
  REGION 1..10: note = CDRH2 of anti-CD3 antibody 4B4-D7
IKARSNNYAT

SEQ ID NO: 56 - protein, 12 aa, synthetic construct
  REGION 1..12: note = CDRH3 of anti-CD3 antibody 4B4-D7
RGTYYASKPFDY

SEQ ID NO: 57 - protein, 11 aa, synthetic construct
  REGION 1..11: note = CDRL1 of anti-CD3 antibody 4E7-C9
QSLEHNNGNTY

SEQ ID NO: 58 - skipped

SEQ ID NO: 59 - protein, 9 aa, synthetic construct
  REGION 1..9: note = CDRL3 of anti-CD3 antibody 4E7-C9
GQGTQYPFT

SEQ ID NO: 60 - protein, 8 aa, synthetic construct
  REGION 1..8: note = CDRH1 of anti-CD3 antibody 4E7-C9
GFTFSNAW

SEQ ID NO: 61 - protein, 10 aa, synthetic construct
  REGION 1..10: note = CDRH2 of anti-CD3 antibody 4E7-C9
IKDKSNNYAT

SEQ ID NO: 62 - protein, 13 aa, synthetic construct
  REGION 1..13: note = CDRH3 of anti-CD3 antibody 4E7-C9
RYVHYGIGYAMDA

SEQ ID NO: 63 - protein, 11 aa, synthetic construct
  REGION 1..11: note = CDRL1 of anti-CD3 antibody 18F5-H10
QSLVHTNGNTY

SEQ ID NO: 64 - skipped

SEQ ID NO: 65 - protein, 9 aa, synthetic construct
  REGION 1..9: note = CDRL3 of anti-CD3 antibody 18F5-H10
GQGTHYPFT

SEQ ID NO: 66 - protein, 8 aa, synthetic construct
  REGION 1..8: note = CDRH1 of anti-CD3 antibody 18F5-H10
GFTFTNAW

SEQ ID NO: 67 - protein, 9 aa, synthetic construct
  REGION 1..9: note = CDRH2 of anti-CD3 antibody 18F5-H10
KDKSNNYAT

SEQ ID NO: 68 - protein, 13 aa, synthetic construct
  REGION 1..13: note = CDRH3 of anti-CD3 antibody 18F5-H10
RYVHYRFAYALDA

SEQ ID NO: 69 - protein, 11 aa, synthetic construct
  REGION 1..11: note = CDRL1 of anti-CD8 antibody OKT8
RTSRSISQYLA

SEQ ID NO: 70 - protein, 7 aa, synthetic construct
  REGION 1..7: note = CDRL2 of anti-CD8 antibody OKT8
SGSTLQS

SEQ ID NO: 71 - protein, 9 aa, synthetic construct
  REGION 1..9: note = CDRL3 of anti-CD8 antibody OKT8
QQHNENPLT

SEQ ID NO: 72 - protein, 6 aa, synthetic construct
  REGION 1..6: note = CDRH1 of anti-CD8 antibody OKT8
GFNIKD

SEQ ID NO: 73 - protein, 9 aa, synthetic construct
  REGION 1..9: note = CDRH2 of anti-CD8 antibody OKT8
RIDPANDNT

SEQ ID NO: 74 - protein, 9 aa, synthetic construct
  REGION 1..9: note = CDRH3 of anti-CD8 antibody OKT8
GYGYYVFDH

SEQ ID NO: 75 - protein, 109 aa, synthetic construct
  REGION 1..109: note = amino acid sequence of a variable light chain region of the sequence of a binding domain that binds and blocks the NK cell inhibitory receptors KIR2DL1 and KIR2DL2/3
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRT

SEQ ID NO: 76 - protein, 123 aa, synthetic construct
  REGION 1..123: note = amino acid sequence of a variable heavy chain region of the sequence of a binding domain that binds and blocks the NK cell inhibitory receptors KIR2DL1 and KIR2DL2/3
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSS

SEQ ID NO: 77 - protein, 11 aa, synthetic construct
  REGION 1..11: note = linker
GGSGGGSGGSG

SEQ ID NO: 78 - protein, 10 aa, synthetic construct
  REGION 1..10: note = linker
GGSGGGSGSG

SEQ ID NO: 79 - protein, 8 aa, synthetic construct
  REGION 1..8: note = linker
GGSGGGSG

SEQ ID NO: 80 - protein, 10 aa, synthetic construct
  REGION 1..10: note = linker
GGGGSGGGGS

SEQ ID NO: 81 - protein, 8 aa, synthetic construct
  REGION 1..8: note = linker
GGGSGGGS

SEQ ID NO: 82 - protein, 6 aa, synthetic construct
  REGION 1..6: note = linker
GGSGGS

SEQ ID NO: 83 - protein, 467 aa, synthetic construct
  REGION 1..467: note = amino acid sequence of the heavy chain of anti-CD33 antibody 1H7
METDTLLLWVLLLWVPGSTGQVQLQQSGAELVKPGASVKISCKASGYAFSNYWMNWVKQRPGKGLEWIGQINPGDGDTNYNGKFKGKATLTADKSSSTAYMQLSSLTSEDSAVYFCAREDRDYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 84 - protein, 234 aa, synthetic construct
  REGION 1..234: note = amino acid sequence of the light chain of anti-CD33 antibody 1H7
METDTLLLWVLLLWVPGSTGDIQMTQTTSSLSASLGDRVTISCRASQDINYYLNWYQQKPDGTVKLLIYYSSRLHSGVPSRFSGSGSGTDFSLTISNLEQEDIATYFCQQDDALPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 85 - protein, 11 aa, synthetic construct
  REGION 1..11: note = CDRL1 of anti-CD33 antibody 1H7
RASQDINYYLN

SEQ ID NO: 86 - protein, 8 aa, synthetic construct
  REGION 1..8: note = CDRL2 of anti-CD33 antibody 1H7
YSSRLHSG

SEQ ID NO: 87 - protein, 9 aa, synthetic construct
  REGION 1..9: note = CDRL3 of anti-CD33 antibody 1H7
QQDDALPYT

SEQ ID NO: 88 - protein, 10 aa, synthetic construct
  REGION 1..10: note = CDRH1 of anti-CD33 antibody 1H7
GYAFSNYWMN

SEQ ID NO: 89 - protein, 13 aa, synthetic construct
  REGION 1..13: note = CDRH2 of anti-CD33 antibody 1H7
QINPGDGDTNYNG

SEQ ID NO: 90 - protein, 8 aa, synthetic construct
  REGION 1..8: note = CDRH3 of anti-CD33 antibody 1H7
EDRDYFDY

SEQ ID NO: 91 - protein, 519 aa, synthetic construct
  REGION 1..519: note = amino acid sequence of a 1H7-CD3 BiTE (based on SEQ ID 83/84)
METDTLLLWVLLLWVPGSTGDIQMTQTTSSLSASLGDRVTISCRASQDINYYLNWYQQKPDGTVKLLIYYSSRLHSGVPSRFSGSGSGTDFSLTISNLEQEDIATYFCQQDDALPYTFGGGTKLEIKGGGGSGGGGSGGGGSQVQLQQSGAELVKPGASVKISCKASGYAFSNYWMNWVKQRPGKGLEWIGQINPGDGDTNYNGKFKGKATLTADKSSSTAYMQLSSLTSEDSAVYFCAREDRDYFDYWGQGTTLTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 92 - protein, 504 aa, synthetic construct
  REGION 1..504: note = amino acid sequence of a light chain 1H7-CD3 engager (based on SEQ ID 84)
METDTLLLWVLLLWVPGSTGDIQMTQTTSSLSASLGDRVTISCRASQDINYYLNWYQQKPDGTVKLLIYYSSRLHSGVPSRFSGSGSGTDFSLTISNLEQEDIATYFCQQDDALPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 93 - protein, 467 aa, synthetic construct; identical to SEQ ID NO: 83
  REGION 1..467: note = amino acid sequence of a heavy chain 1H7-CD3 engager (based on SEQ ID 83)

SEQ ID NO: 94 - protein, 10 aa, synthetic construct
  REGION 1..10: note = CDRH1 of anti-CD33 antibody 5D12
GYAFSNYWMN

SEQ ID NO: 95 - protein, 17 aa, synthetic construct
  REGION 1..17: note = CDRH2 of anti-CD33 antibody 5D12
QIYPGNFNTDYNGQFKG

SEQ ID NO: 96 - protein, 12 aa, synthetic construct
  REGION 1..12: note = CDRH3 of anti-CD33 antibody 5D12
FFDFGAYFTLDY

SEQ ID NO: 97 - protein, 11 aa, synthetic construct
  REGION 1..11: note = CDRL1 of anti-CD33 antibody 5D12
KASQDIKSYLS

SEQ ID NO: 98 - protein, 7 aa, synthetic construct
  REGION 1..7: note = CDRL2 of anti-CD33 antibody 5D12
YATTLAD

SEQ ID NO: 99 - protein, 9 aa, synthetic construct
  REGION 1..9: note = CDRL3 of anti-CD33 antibody 5D12
LHHGESPWT

SEQ ID NO: 100 - protein, 11 aa, synthetic construct
  REGION 1..11: note = CDRH1 of anti-CD33 antibody 8F5
SGFTFSDFYMY

SEQ ID NO: 101 - protein, 17 aa, synthetic construct
  REGION 1..17: note = CDRH2 of anti-CD33 antibody 8F5
FISNAGVTTYYPDTVEG

SEQ ID NO: 102 - protein, 10 aa, synthetic construct
  REGION 1..10: note = CDRH3 of anti-CD33 antibody 8F5
SDYDGAWFPY

SEQ ID NO: 103 - protein, 17 aa, synthetic construct
  REGION 1..17: note = CDRL1 of anti-CD33 antibody 8F5
KSSQSLLYSRNQYNFLA

SEQ ID NO: 104 - protein, 7 aa, synthetic construct
  REGION 1..7: note = CDRL2 of anti-CD33 antibody 8F5
WASTRES

SEQ ID NO: 105 - protein, 9 aa, synthetic construct
  REGION 1..9: note = CDRL3 of anti-CD33 antibody 8F5
QQYYSYPYT

SEQ ID NO: 106 - protein, 10 aa, synthetic construct
  REGION 1..10: note = CDRH1 of anti-CD33 antibody 12B12
GYTFTTYWMH

SEQ ID NO: 107 - protein, 13 aa, synthetic construct
  REGION 1..13: note = CDRH2 of anti-CD33 antibody 12B12
AIYPGNSDTSYNQ

SEQ ID NO: 108 - protein, 7 aa, synthetic construct
  REGION 1..7: note = CDRH3 of anti-CD33 antibody 12B12
YDGYHFI

SEQ ID NO: 109 - protein, 16 aa, synthetic construct
  REGION 1..16: note = CDRL1 of anti-CD33 antibody 12B12
RSSQSLLHSNGITYLY

SEQ ID NO: 110 - protein, 7 aa, synthetic construct
  REGION 1..7: note = CDRL2 of anti-CD33 antibody 12B12
QMSNLAS

SEQ ID NO: 111 - protein, 9 aa, synthetic construct
  REGION 1..9: note = CDRL3 of anti-CD33 antibody 12B12
AQNLELPPT

SEQ ID NO: 112 - protein, 10 aa, synthetic construct
  REGION 1..10: note = CDRH1 of anti-CD33 antibody 4H10
GYAFTNYLIE

SEQ ID NO: 113 - protein, 13 aa, synthetic construct
  REGION 1..13: note = CDRH2 of anti-CD33 antibody 4H10
VIHPGNNSTSYNA

SEQ ID NO: 114 - protein, 11 aa, synthetic construct
  REGION 1..11: note = CDRH3 of anti-CD33 antibody 4H10
YGYDERNAMDY

SEQ ID NO: 115 - protein, 13 aa, synthetic construct
  REGION 1..13: note = CDRL1 of anti-CD33 antibody 4H10
QSLLYSNGKTYLH

SEQ ID NO: 116 - protein, 16 aa, synthetic construct
  REGION 1..16: note = CDRL2 of anti-CD33 antibody 4H10
VPDRFTGSGSGTDFTL

SEQ ID NO: 117 - protein, 9 aa, synthetic construct
  REGION 1..9: note = CDRL3 of anti-CD33 antibody 4H10
GTHFPRTFG

SEQ ID NO: 118 - protein, 10 aa, synthetic construct
  REGION 1..10: note = CDRH1 of anti-CD33 antibody 11D5
GYTFTSYWMH

SEQ ID NO: 119 - protein, 13 aa, synthetic construct
  REGION 1..13: note = CDRH2 of anti-CD33 antibody 11D5
AIYCGNSDTSYNQ

SEQ ID NO: 120 - protein, 8 aa, synthetic construct
  REGION 1..8: note = CDRH3 of anti-CD33 antibody 11D5
YDGYHFDY

SEQ ID NO: 121 - protein, 16 aa, synthetic construct
  REGION 1..16: note = CDRL1 of anti-CD33 antibody 11D5
RSNKSLLHSNGITYLY

SEQ ID NO: 122 - protein, 7 aa, synthetic construct
  REGION 1..7: note = CDRL2 of anti-CD33 antibody 11D5
QMSNLAS

SEQ ID NO: 123 - protein, 9 aa, synthetic construct
  REGION 1..9: note = CDRL3 of anti-CD33 antibody 11D5
AQNLELPPT

SEQ ID NO: 124 - protein, 10 aa, synthetic construct
  REGION 1..10: note = CDRH1 of anti-CD33 antibody 13E11
GYTFTDYTLH

SEQ ID NO: 125 - protein, 13 aa, synthetic construct
  REGION 1..13: note = CDRH2 of anti-CD33 antibody 13E11
WFYPTSGSINYNE

SEQ ID NO: 126 - protein, 7 aa, synthetic construct
  REGION 1..7: note = CDRH3 of anti-CD33 antibody 13E11
HKFGFDY

SEQ ID NO: 127 - protein, 15 aa, synthetic construct
  REGION 1..15: note = CDRL1 of anti-CD33 antibody 13E11
KASHGVEYAGAHYMN

SEQ ID NO: 128 - protein, 7 aa, synthetic construct
  REGION 1..7: note = CDRL2 of anti-CD33 antibody 13E11
AASNLGS

SEQ ID NO: 129 - protein, 9 aa, synthetic construct
  REGION 1..9: note = CDRL3 of anti-CD33 antibody 13E11
QQSNEDPRT

SEQ ID NO: 130 - protein, 10 aa, synthetic construct
  REGION 1..10: note = CDRH1 of anti-CD33 antibody 5E10.7
GYTFTSYWMQ

SEQ ID NO: 131 - protein, 13 aa, synthetic construct
  REGION 1..13: note = CDRH2 of anti-CD33 antibody 5E10.7
EIDSSDSYTNYNQ

SEQ ID NO: 132 - protein, 9 aa, synthetic construct
  REGION 1..9: note = CDRH3 of anti-CD33 antibody 5E10.7
SRPYDWFPY

SEQ ID NO: 133 - protein, 11 aa, synthetic construct
  REGION 1..11: note = CDRL1 of anti-CD33 antibody 5E10.7
KASQDIKSYLS

SEQ ID NO: 134 - protein, 7 aa, synthetic construct
  REGION 1..7: note = CDRL2 of anti-CD33 antibody 5E10.7
YATTLAD

SEQ ID NO: 135 - protein, 9 aa, synthetic construct
  REGION 1..9: note = CDRL3 of anti-CD33 antibody 5E10.7
LHHSESPWT

SEQ ID NO: 136 - protein, 11 aa, synthetic construct
  REGION 1..11: note = CDRH1 of anti-CD33 antibody 11D11
SGFTFSDFYME

SEQ ID NO: 137 - protein, 13 aa, synthetic construct
  REGION 1..13: note = CDRH2 of anti-CD33 antibody 11D11
ASRNKANDYTTEY

SEQ ID NO: 138 - protein, 7 aa, synthetic construct
  REGION 1..7: note = CDRH3 of anti-CD33 antibody 11D11
DTGPMDY

SEQ ID NO: 139 - protein, 11 aa, synthetic construct
  REGION 1..11: note = CDRHL1 of anti-CD33 antibody 11D11
RASQDISNYLN

SEQ ID NO: 140 - protein, 7 aa, synthetic construct
  REGION 1..7: note = CDRHL2 of anti-CD33 antibody 11D11
YTSRLHS

SEQ ID NO: 141 - protein, 9 aa, synthetic construct
  REGION 1..9: note = CDRHL3 of anti-CD33 antibody 11D11
QQGSTLPPT

SEQ ID NO: 142 - protein, 12 aa, synthetic construct
  REGION 1..12: note = CDRH1 of anti-CD33 antibody 7E7
GFSLNSYGMGIG

SEQ ID NO: 143 - protein, 16 aa, synthetic construct
  REGION 1..16: note = CDRH2 of anti-CD33 antibody 7E7
HIWWDDNKYYKPDLKS

SEQ ID NO: 144 - protein, 9 aa, synthetic construct
  REGION 1..9: note = CDRH3 of anti-CD33 antibody 7E7
DGGYSLFAY

SEQ ID NO: 145 - protein, 16 aa, synthetic construct
  REGION 1..16: note = CDRL1 of anti-CD33 antibody 7E7
KSSQSLLDSDGKTYLS

SEQ ID NO: 146 - protein, 7 aa, synthetic construct
  REGION 1..7: note = CDRL2 of anti-CD33 antibody 7E7
VSKLDSG

SEQ ID NO: 147 - protein, 9 aa, synthetic construct
  REGION 1..9: note = CDRL3 of anti-CD33 antibody 7E7
WQGTHFPLT

SEQ ID NO: 148 - protein, 10 aa, Mus musculus
GYTFTDYDMH

SEQ ID NO: 149 - protein, 15 aa, Mus musculus
AIDPETGGTAYNQNF

SEQ ID NO: 150 - protein, 7 aa, Mus musculus
DYDYFGV

SEQ ID NO: 151 - protein, 11 aa, Mus musculus
KASQNVGTNVV

SEQ ID NO: 152 - protein, 7 aa, Mus musculus
SASDRYS

SEQ ID NO: 153 - protein, 9 aa, Mus musculus
QQYNIYPYT

SEQ ID NO: 154 - protein, 128 aa, Mus musculus
MESQTQVFVYMLLWLSGVDGDIVMTQSQKFMSTSVGDRVSVTCKASQNVGTNVVWYHKKPGQSPKGLIYSASDRYSGVPDRFTGSGSGTDFTLTINNVQSEDLAEYFCQQYNIYPYTFGGGTKLEIKR

SEQ ID NO: 155 - protein, 135 aa, Mus musculus
MEWSWVCLFLLSVIAGVQSQVQLQQSGAELVRPGASVTLSCKASGYTFTDYDMHWVKQTPVHGLEWIGAIDPETGGTAYNQNFKGKAILTVDKSSRIAYMELRSLTSEDSAVFYCTSDYDYFGVWGTGTTVTVSS

SEQ ID NO: 156 - protein, 44 aa, synthetic construct
  REGION 1..44: note = engingeered protein containing signal peptide from full length CD33 fused to c-terminal end of CD33deltaE2
MPLLLLLPLLWAGALAMDLTHRPKILIPGTLEPGHSKNLTCSVS

SEQ ID NO: 157 - protein, 48 aa, synthetic construct
  REGION 1..48: note = engingeered protein containing signal peptide from CD33deltaE2 fused to c-terminal end of full length CD33
MPLLLLLPLLWADLTHRPKILIPNFWLQVQESVTVQEGLCVLVPCTFF

SEQ ID NO: 158 - protein, 15 aa, synthetic construct
  REGION 1..15: note = glycine-serine linker
GGGGSGGGGSGGGGS

SEQ ID NO: 159 - protein, 5 aa, synthetic construct
  REGION 1..5: note = glycine-serine linker
GGGGS

(19) United States
(12) Patent Application Publication
(10) Pub. No.: US 2025/0034250 A1
(43) Pub. Date: Jan. 30, 2025

(54) ANTI-CD33 ANTIBODIES AND USES THEREOF

(71) Applicant: Fred Hutchison Cancer Center, Seattle, WA (US)

(72) Inventors: Roland B. Walter, Seattle, WA (US); Christopher Mehlin, Seattle, WA (US); George S. Laszlo, Seattle, WA (US); Colin E. Correnti, Seattle, WA (US)

(73) Assignee: Fred Hutchinson Cancer Center, Seattle, WA (US)

(21) Appl. No.: 18/913,796

(22) Filed: Oct. 11, 2024

Related U.S. Application Data
(62) Division of application No. 16/617,445, filed on Nov. 26, 2019, now Pat. No. 12,116,406.
(60) Provisional application No. 62/532,772, filed on Jul. 14, 2017.
(60) Provisional application No. 62/511,792, filed on May 26, 2017.

(51) Int. Cl. C07K 16/28; A61P 35/02

(52) U.S. Cl. CPC C07K 16/2803; A61P 35/02; C07K 2317/31; C07K 2317/73; C07K 2317/92

(57) ABSTRACT

Anti-CD33 antibodies are described. The anti-CD33 antibodies can bind within the V-set Ig-like domain or the C2-set Ig-like domain of CD33. Epitopes on the C2-set Ig-like domain can provide a “pan-binding” site, to which the cognate antibody will bind regardless of whether the CD33 molecule also contains the V-set domain (as in, for example, CD33^{FL}) or not (as in, for example, CD33^{ΔE2}). Alternative epitopes on the C2-set Ig-like domain are accessible for binding if the V-set domain is absent (e.g., as in CD33^{ΔE2}). Antibodies that bind an epitope on the C2-set Ig-like domain (whether they exhibit pan or V-set absent binding) are directed at novel therapeutic targets and can increase therapeutic efficacy against CD33-related disorders. Also described are molecules comprising a binding-competent domain from at least one described anti-CD33 antibody, including scFvs, bi-specific antibody molecules, chimeric antigen receptors, and immunoconjugates. Methods of use are also provided.

### CROSS-REFERENCE TO RELATED APPLICATIONS

This application is a divisional of U.S. patent application Ser. No. 16/617,445, filed on Nov. 26, 2019, which is a U.S. National Stage application based on International Patent Application No. PCT/US2018/034743, filed on May 25, 2018, which claims priority to U.S. Provisional Patent Application No. 62/511,792 filed May 26, 2017, and to U.S. Provisional Patent Application No. 62/532,772 filed Jul. 14, 2017, each of which is incorporated herein by reference in its entirety as if fully set forth herein.

### STATEMENT REGARDING FEDERALLY SPONSORED RESEARCH OR DEVELOPMENT

This invention was made with government support under grant CA100632 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 3B35191.XML. The text file is 212,992 bytes, was created on Sep. 27, 2024, and is being submitted electronically via Patent Center.

### FIELD OF THE DISCLOSURE

Anti-CD33 antibodies are described. The antibodies can bind CD33 proteins containing the V-set Ig-like domain or CD33 proteins containing the C2-set Ig-like domain (alone or together with V-set domain). Antibodies that bind the C2-set Ig-like domain (for instance, in CD33^{ΔE2} or in both CD33^{FL} and CD33^{ΔE2}) are directed at novel therapeutic targets and can increase the therapeutic efficacy against CD33-expressing disorders.

### BACKGROUND OF THE DISCLOSURE

There are ˜20,000 new cases of acute myeloid leukemia (AML) per year in the United States. Treatment outcomes remain unsatisfactory for many, with a 5-year relative survival of only 40% for patients younger than 45 years of age and only 5% for patients >65 years of age. The median age at diagnosis of AML is 67 years.

CD33 is a member of the sialic acid binding, immunoglobulin-like lectin protein family. It is a 67-kDa glycosylated transmembrane protein. Full-length CD33 (CD33^{FL}) is a myeloid differentiation antigen that is found at least on some leukemic cells in almost all patients with acute myeloid leukemia (AML) and, perhaps, on AML stem cells in some cases. Based on this broad expression pattern, CD33^{FL} has been widely pursued as a therapeutic target in AML. Recent data from several randomized studies have demonstrated that the CD33 antibody-drug conjugate, gemtuzumab ozogamicin (GO), improves survival when added to chemotherapy in defined subsets of patients with newly diagnosed AML. This data has validated CD33^{FL} as the first (and so far, only) target for immunotherapy in AML.

CD33^{FL} includes a V-set Ig-like domain and a C2-set Ig-like domain (see FIGS. 1A, 1C, and 2). At the mRNA level, a splice variant of CD33 that lacks exon 2 (CD33^{ΔE2}) has also been identified (see FIG. 1B). However, commercial antibodies that are currently available recognize immune-dominant epitope(s) on the V-set Ig-like domain that is encoded by exon 2 of the CD33gene. Because of that, CD33^{ΔE2} and other CD33 proteins that lack the V-set domain are currently not recognized by any available antibody.

### SUMMARY OF THE DISCLOSURE

The current disclosure provides antibodies that bind/recognize 1) the V-set domain of CD33 (including full-length CD33, CD33^{FL}; see FIG. 1A); 2) the C2-set domain only in CD33 proteins that lack the V-set domain (e.g., CD33^{ΔE2} is an example of such a CD33 protein; see FIG. 1B); and 3) the C2-set domain in CD33 proteins regardless of whether or not the V-set domain is present (e.g. can recognize CD33^{FL} and CD33^{ΔE2}; see FIG. 1C). The disclosed antibodies can be used alone or in various combinations to target unwanted CD33-expressing cells (e.g., CD33-expressing cancer cells). In combination, the disclosed antibodies can target a higher percentage of CD33-expressing cells because they can bind target cells expressing CD33 variants (such as CD33^{ΔE2}) as well as CD33^{FL}. Further, the development of antibodies specifically binding CD33 only if the V-set domain is not present (e.g., CD33^{ΔE2}) provides therapeutic targeting for cells that express variants, but that do not express protein(s) that contain the V-set domain (e.g., CD33^{FL}).

### BRIEF DESCRIPTION OF THE SEVERAL VIEWS OF THE DRAWINGS

Many of the drawings submitted herein are better understood in color, which is not available in patent application publications at the time of filing. Applicant considers the color versions of the drawings as part of the original submission and reserve the right to present color images of the drawings in later proceedings.

FIGS. 1A-1C. Diagrams schematically illustrating binding of anti-CD33 antibodies that bind different domains of CD33 and related isoforms. FIG. 1A illustrates an example of an antibody that binds the V-set domain. FIG. 1B illustrates an example of an antibody that binds a novel epitope presented by the CD33ΔE2 isoform. FIG. 1C. illustrates an example of an antibody that binds the C2-set domain in the context of the full-length receptor or the CD33ΔE2 receptor.

FIG. 2. Sequence alignment of human, murine and cynomolgus monkey (cyno) CD33.Sequences are annotated to show secretion signals, domains, disulfide pairing and transmembrane regions: N-linked glycosylation sites are shown with asterisks. Cysteines that form disulfide bonds are shown with bars. Conserved disulfide bonds are formed between residues 41 and 101, 36 and 170, and 164 and 213; numbered as in the alignment.

FIGS. 3A-3D. CD33^{ΔE2} Neoepitope. Extracellular versions of CD33^{FL} (SEQ ID NO: 5) and the CD33^{ΔE2} (SEQ ID NO: 7) isoforms were expressed, purified, and characterized. Size exclusion chromatography (FIG. 3A) and SDS-PAGE gel (FIG. 3B) analyses show the purified recombinant proteins. FIG. 3C shows signal peptide prediction using the program SignalP. The sequence shown in FIG. 3C corresponds to positions 1-70 of SEQ ID NO: 2. FIG. 3D shows peptide sequences (SEQ ID NOs: 28-31) derived from the N-terminus of the recombinant CD33^{ΔE2} isoform as determined by mass spectrometry. Recombinant protein was reduced, alkylated and trypsinized to generate the data set.

FIGS. 4A-4C. Expression and characterization of two V-set binding (detected on CD33^{FL}) and one C2-set binding (detected on CD33^{ΔE2}) specific antibodies. Affinities are characterized by Biacore using purified antibodies and the corresponding soluble ectodomains.

FIG. 5. CD33 V-set domain binding antibodies. Flow cytometry binding assay showing antibodies that bind CD33^{FL} compared with P67.6. Most, if not all, therapeutic and diagnostic antibodies are specific for the membrane distal V-set Ig-like domain of CD33 and therefore do not recognize the C2-set Ig-like domain and consequently also do not recognize the CD33^{ΔE2} isoform. REH=human CD33^{neg} lymphoid cells.

FIG. 6. CD33 C2-set Ig-like domain specific CD33 antibodies. Binding assay showing novel antibodies that bind the CD33^{ΔE2} isoform and not CD33^{FL}. Antibodies specific for CD33^{ΔE2} have not been described reliably in the literature and will be useful for determining expression profiles of this isoform across healthy and diseased individuals. REH=human CD33^{neg} lymphoid cells.

FIG. 7. “Pan-specific” CD33 antibody. Binding assay showing novel antibodies that bind the C2-set Ig-like domain and therefore recognize both CD33^{FL} and CD33^{ΔE2} , as well as other naturally occurring splice isoforms (e.g., CD33^{Δ7a}, CD33^{ΔE2,7a}). These antibodies and others with similar specificities are superior to those described in the literature because (1) they can target a higher density of CD33 antigen and (2) they bind a membrane proximal epitope which is predicted to yield enhanced antibody effector functions, for example, increased activity of immune cell-engaging bispecific antibodies and chimeric antigen receptor (CAR)-modified immune cells, which could also benefit from a membrane proximal epitope. REH=human CD33^{neg} lymphoid cells.

FIG. 8. Binding of commercial V-set domain CD33 antibody (P67.6) as well as in-house-made C2-set domain CD33PAN antibodies to parental (CD33-negative) acute leukemia cell line (REH cells; black) and REH sublines expressing either CD33^{FL} (grey) or CD33^{ΔE2} (dashed).

FIG. 9. Schemes of naturally-occurring CD33 isoforms and artificial CD33 proteins to characterize CD33 antibodies in this application.

FIG. 10. Cytotoxicity of CD33PAN/CD3 bispecific antibody and AMG 330 (a previously published investigational V-set domain-directed CD33/CD3 bispecific T-cell engager) on parental (CD33-negative) REH cells (black) and REH cells transduced with CD33^{FL} (grey) or CD33^{ΔE2} (cross hatched); mean±SEM (n=4).

FIG. 11. Cytotoxicity of CD33FL/CD3 bispecific antibody AMG 330 on parental (CD33-negative) RS4;11 cells (black) and RS4;11 cells transduced with CD33^{FL} (grey) or CD33^{ΔE3-4} (cross hatched); mean±SEM (n=3).

FIGS. 12A-12B. Schematic showing the format of the 1H7 bispecific antibody T-cell engager (FIG. 12A), along with the amino acid sequence and SDS-PAGE gel of purified product (FIG. 12B). In FIG. 12A, (G4S) 3 is SEQ ID NO: 158 and G4S is SEQ ID NO: 159.

FIGS. 13A-13B. Schematic showing the format of the 1H7 Ig-bispecific antibody T-cell engager (FIG. 13A), along with the amino acid sequence of the light and heavy chains and SDS-PAGE gel of purified product (FIG. 13B).

FIGS. 14A-14C. Schematic showing signal peptide cleavage of naturally-occurring CD33 isoforms and engineered hybrid isoforms (14A), along with characterization of two unique CD33 C2-set Ig-like domain specific CD33 antibodies with unique specificities for CD33^{ΔE2} protein. In order, the sequences shown in FIG. 14A correspond to:
residues 1-44 of SEQ ID NO: 1 (exon 1=positions 1-12);
residues 1-39 of SEQ ID NO: 2 (exon 1=positions 1-12);
SEQ ID NO: 156 (exon 1=positions 1-12);
SEQ ID NO: 157 (exon 1=positions 1-12);
residues 18-44 of SEQ ID NO: 1;
residues 1-39 of SEQ ID NO: 2 (exon 1=positions 1-12);
residues 140-166 of SEQ ID NO: 1 (which is also 13-39 of SEQ ID NO: 2); and
SEQ ID NO: 157 (exon 1=positions 1-12).

### DETAILED DESCRIPTION

There are 20,000 new cases of acute myeloid leukemia (AML) per year in the United States. Treatment outcomes remain unsatisfactory for many, with a 5-year relative survival of only 40% for patients younger than 45 years of age and only 5% for patients >65 years of age. The median age at diagnosis of AML is 67 years.

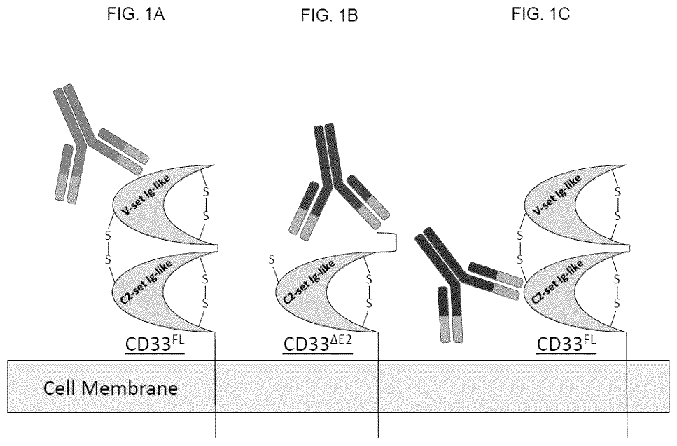

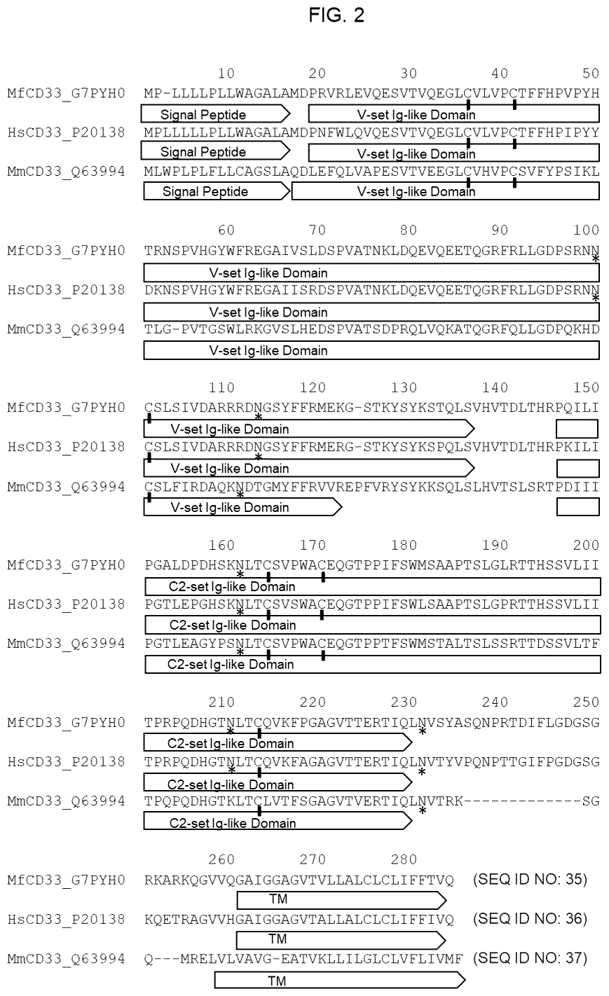

CD33^{FL} is a transmembrane glycoprotein that is characterized by an amino-terminal, membrane-distant V-set immunoglobulin (Ig)-like domain and a membrane-proximal C2-set Ig-like domain in its extracellular portion (see FIGS. 1A, 1B, and 2). CD33^{FL} is primarily displayed on maturing and mature cells of the myeloid lineage, with initial expression on multipotent myeloid precursors. It is not found outside the hematopoietic system and is not thought to be expressed on pluripotent hematopoietic stem cells. Consistent with its role as a myeloid differentiation antigen, CD33^{FL} is widely expressed on malignant cells in patients with myeloid neoplasms; e.g., in acute myeloid leukemia (AML), it is found on at least a subset of the AML blast cells in almost all cases and possibly leukemic stem cells in some. Because of this expression pattern, CD33^{FL} has been widely exploited as an antigen for targeted therapy of AML. (Walter et al., Blood 119(26): 6198-6208, 2012; Cowan et al., Front. Biosci. (Landmark Ed) 18:1311-1334, 2013; Laszlo et al., Blood Ref. 28 (4): 143-153, 2014. ) While unconjugated monoclonal CD33 antibodies proved ineffective in the clinic, several recent randomized trials with the CD33 antibody-drug conjugate (ADC) gemtuzumab ozogamicin (GO) have demonstrated improved survival in subsets of patients with AML, establishing the value of antibodies in this disease and validating CD33^{FL} as the first, and so far only, therapeutic target for immunotherapy of AML (Laszlo et al., Blood Rev. 28(4): 143-153, 2014; Godwin et al., Leukemia 31(9) 31 (9): 1855-1868, 2017).

In addition to CD33^{FL}, a splice variant that misses exon 2 (CD331E2) has also been identified at the mRNA level in normal hematopoietic cells as well as leukemia cells. Regarding the latter, CD33^{ΔE2} mRNA was identified in 29 of 29 tested AML patient specimens, indicating universal expression in human AML. CD33^{ΔE2} contains the C2-set Ig-like domain but not the V-set Ig-like domain of CD33 (see FIG. 1B). Since all commercial diagnostic CD33 antibodies and current CD33 antibody-based therapeutics recognize the immune-dominant V-set Ig-like domain that is encoded by exon 2 (see FIG. 1A), CD33^{ΔE2} and other CD33 proteins that lack the V-set domain are not recognized by any available CD33 antibody (previous studies suggested that one CD33 antibody [clone Him3-4] specifically recognizes the C2-set Ig-like domain, but other reports indicate Him3-4 only recognizes CD33^{FL}; Laszlo et al., Oncotarget 7 (28): 43281-43294, 2016). Additional splice variants, identified at the mRNA level, include CD33^{ΔE7a} and CD334E2/E7a. CD33^{ΔE7a} uses an alternate exon 7 (E7a) which results in a truncation of the intracellular domain of CD33. CD33^{ΔE2/E7a} lacks exon 2 and also has the truncation of the intracellular domain of CD33. Other CD33 proteins may exist naturally.

Antibodies that recognize only CD33 proteins that lack the V-set domain (the CD33^{ΔE2} isoform being one example) (FIG. 1B) provide a novel therapeutic approach as such isoform can provide a “stand-alone” isoform-specific therapeutic target, which current CD33 antibodies are unable to bind. Additionally, antibodies that recognize both bind the C2-set domain and recognize CD33 proteins regardless of the presence/absence of the V-set domain (e.g. antibodies that bind the CD33^{ΔE2} and CD33^{FL} isoforms) (FIG. 1C; “pan” antibodies) can provide better anti-CD33 effects than current CD33 antibodies because (1) they recognize a higher target antigen density due to binding to both isoforms, and/or (2) they bind closer to the membrane. For several therapeutic targets, the specifics of the targeted epitope have been shown to be critically important for antibody-based therapy, with membrane-proximal epitopes resulting in more potent anti-tumor effects than membrane-distal ones, as shown for CD20, CD22, CD25, and EpCAM. See, for instance, Cleary et al., J Immunol. 2017;198 (10): 3999-4011; Lin, Pharmgenomics Pers Med. 2010;3:51-59; Haso et al., Blood. 2013;121 (7): 1165-1174; and Bluemel et al., Cancer Immunol Immunother. 2010;59 (8): 1197-1209.

In particular embodiments, antibodies disclosed herein bind to CD33 and have one or more of the following characteristics:
(a) bind to recombinant human CD33;
(b) bind to recombinant cynomolgus monkey CD33;
(c) bind to endogenous CD33 on the surface of human peripheral blood mononuclear cells (PBMCs);
(d) bind to endogenous CD33 on the surface of cynomolgus monkey PBMCs;
(e) bind to endogenous CD33 on the surface of a cancer cell;
(f) bind to endogenous CD33 on the surface of an AML cancer cell;
(g) bind to an epitope within the CD33 V-set Ig-like domain;
(h) bind to an epitope within the CD33 C2-set Ig-like domain in the presence of a V-set Ig-like domain (e.g., in CD33^{FL});
(i) bind to an epitope within the CD33 C2-set Ig-like domain in the absence of a V-set Ig-like domain (e.g., in CD33^{ΔE2});
(j) bind to a neoepitope on CD33^{ΔE2} due to a retained signal peptide on the amino terminal of CD33^{ΔE2}.
(k) bind to a membrane proximal epitope of CD33;
(l) include a VL chain of SEQ ID NO: 8 and a VH chain of SEQ ID NO: 9;
(m) include a VL chain of SEQ ID NO: 10 and a VH chain of SEQ ID NO: 11;
(n) include a VL chain of SEQ ID NO: 12 and a VH chain of SEQ ID NO: 13;
(o) include a VL chain of SEQ ID NO: 14 and a VH chain of SEQ ID NO: 15;
(p) include a VL chain of SEQ ID NO: 16 and a VH chain of SEQ ID NO: 17;
(q) include a VL chain of SEQ ID NO: 18 and a VH chain of SEQ ID NO: 19;
(r) include a VL chain of SEQ ID NO: 20 and a VH chain of SEQ ID NO: 21;
(s) include a VL chain of SEQ ID NO: 22 and a VH chain of SEQ ID NO: 23;
(t) include a VL chain of SEQ ID NO: 24 and a VH chain of SEQ ID NO: 25;
(u) include a VL chain of SEQ ID NO: 84 and a VH chain of SEQ ID NO: 83;
(v) include a VL chain of SEQ ID NO: 154 and a VH chain of SEQ ID NO: 155;
(w) include CDRs including SEQ ID NO: 85; SEQ ID NO: 86; SEQ ID NO: 87; SEQ ID NO: 88; SEQ ID NO: 89; and SEQ ID NO: 90;
(x) include CDRs including SEQ ID NO: 130; SEQ ID NO: 131; SEQ ID NO: 132; SEQ ID NO: 133; SEQ ID NO: 134; and SEQ ID NO: 135;
(y) include CDRs including SEQ ID NO: 106, SEQ ID NO: 107, SEQ ID NO: 108, SEQ ID NO: 109, SEQ ID NO: 110, and SEQ ID NO: 111;
(z) include CDRs including SEQ ID NO: 112, SEQ ID NO: 113, SEQ ID NO: 114, SEQ ID NO: 115, SEQ ID NO: 116, and SEQ ID NO: 117;
(aa) include CDRs including SEQ ID NO: 118, SEQ ID NO: 119, SEQ ID NO: 120, SEQ ID NO: 121, SEQ ID NO: 122, and SEQ ID NO: 123;
(bb) include CDRs including SEQ ID NO: 124, SEQ ID NO: 125, SEQ ID NO: 126, SEQ ID NO: 127, SEQ ID NO: 128, and SEQ ID NO: 129;
(cc) include CDRs including SEQ ID NO: 136, SEQ ID NO: 137, SEQ ID NO: 138, SEQ ID NO: 139, SEQ ID NO: 140, and SEQ ID NO: 141;
(dd) include CDRs including SEQ ID NO: 142, SEQ ID NO: 143, SEQ ID NO: 144, SEQ ID NO: 145, SEQ ID NO: 146, and SEQ ID NO: 147
(ee) include CDRs including SEQ ID NO: 94, SEQ ID NO: 95, SEQ ID NO: 96, SEQ ID NO: 97, SEQ ID NO: 98, and SEQ ID NO: 99;
(ff) include CDRs including SEQ ID NO: 100, SEQ ID NO: 101, SEQ ID NO: 102, SEQ ID NO: 103, SEQ ID NO: 104, and SEQ ID NO: 105;
(gg) include CDRs including SEQ ID NO: 148, SEQ ID NO: 149, SEQ ID NO: 150, SEQ ID NO: 151, SEQ ID NO: 152, and SEQ ID NO: 153;
(hh) includes a humanized sequence of an antibody disclosed herein; and/or
(ii) binds to endogenous human CD33 with a Kp of less than 10 nM.

Having highlighted key aspects of the current disclosure, the following more detailed description is now provided.

CD33 refers to any native, mature CD33 which results from processing of a CD33 precursor protein in a cell. A CD33-positive cell refers to any cell that expresses CD33 on its surface. A CD33-positive cancer refers to a cancer including one or more cells that express CD33 on their surface. Examples of CD33-positive cancers include leukemia, myeloid sarcoma, and lymphoma (e.g., Hodgkin's and non-Hodgkin's lymphoma). More particular examples of such cancers include acute myeloid leukemia (AML), myelodysplastic syndrome (MDS), chronic myelogenous leukemia (CML), chronic myelomonocytic leukemia (CMML), acute promyelocytic leukemia (APL), myeloproliferative neoplasms, megakaryocytic leukemia, B-cell acute lymphoblastic leukemia (B-ALL), T-cell acute lymphoblastic leukemia (T-ALL), multiple myeloma (MM) and other plasma cell dyscrasias, mast cell disease, mast cell leukemia, mast cell sarcoma, myeloid sarcomas, lymphoid leukemia, and undifferentiated leukemia.

The current disclosure provides antibodies against various CD33 epitopes.

Naturally occurring antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md., 1987 and 1991), or Chothia & Lesk, J. Mol. Biol., 196:901-917, 1987; Chothia et al., Nature, 342:878-883, 1989.

Definitive delineation of a CDR and identification of residues including the binding site of an antibody can be accomplished by solving the structure of the antibody and/or solving the structure of the antibody-epitope complex. In particular embodiments, this can be accomplished by methods such as X-ray crystallography. Alternatively, CDRs are determined by comparison to known antibodies (linear sequence) and without resorting to solving a crystal structure. To determine residues involved in binding, a co-crystal structure of the Fab (antibody fragment) bound to the target can optionally be determined.

The carboxy-terminal portion of each chain defines a constant region, which can be responsible for effector function particularly in the heavy chain (the Fc). Examples of effector functions include: C1q binding and complement dependent cytotoxicity (CDC); antibody- dependent cell-mediated cytotoxicity (ADCC); phagocytosis; down regulation of cell surface receptors (e.g. B-cell receptors); and B-cell activation.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2.

As indicated, antibodies bind epitopes on antigens. The term antigen refers to a molecule or a portion of a molecule capable of being bound by an antibody. An epitope is a region of an antigen that is bound by the variable region of an antibody. Epitope determinants can include chemically active surface groupings of molecules such as amino acids, sugar side chains, phosphoryl or sulfonyl groups, and can have specific three-dimensional structural characteristics, and/or specific charge characteristics. When the antigen is a protein or peptide, the epitope includes specific amino acids within that protein or peptide that contact the variable region of an antibody.

In particular embodiments, an epitope denotes the binding site on CD33 bound by a corresponding variable region of an antibody. The variable region either binds to a linear epitope, (e.g., an epitope including a stretch of 5 to 12 consecutive amino acids), or the variable region binds to a three-dimensional structure formed by the spatial arrangement of several short stretches of the protein target. Three-dimensional epitopes recognized by a variable region, e.g. by the epitope recognition site or paratope of an antibody or antibody fragment, can be thought of as three-dimensional surface features of an epitope molecule. These features fit precisely (in) to the corresponding binding site of the variable region and thereby binding between the variable region and its target protein (more generally, antigen) is facilitated. In particular embodiments, an epitope can be considered to have two levels: (i) the “covered patch” which can be thought of as the shadow an antibody variable region would cast on the antigen to which it binds; and (ii) the individual participating side chains and backbone residues that facilitate binding. Binding is then due to the aggregate of ionic interactions, hydrogen bonds, and hydrophobic interactions.

Epitopes of the currently disclosed antibodies (that is, epitopes to which the antibodies bind) are found within the V-set Ig-like domain or the C2-set Ig-like domain of CD33. When present on the C2-set Ig-like domain, the epitope can provide a “pan binding” site, meaning that the antibody will bind regardless of whether the CD33 molecule also contains the V-set domain (as in, for example, is CD33^{FL}) or not (as in, for example, CD33^{ΔE2}) (FIG. 1C). Alternatively, the epitope on the C2-set Ig-like domain in some embodiments can be only accessible for binding if the V-set domain is absent as in, for example, in the CD33^{ΔE2} splice variant (FIG. 1B). In particular embodiments, epitopes on the C2-set Ig-like domain are membrane-proximal epitopes. In particular embodiments, membrane membrane-proximal epitopes are within 115 residues of the transmembrane region; within 100 residues of the transmembrane region; within 75 residues of the transmembrane region; within 50 residues of the CD33^{PAN} transmembrane region; within 25 residues of the transmembrane region or within 15 residues of the transmembrane region (see FIG. 2). Epitopes within the V-set Ig-like domain are also targeted by certain antibodies of the current disclosure (FIG. 1A).

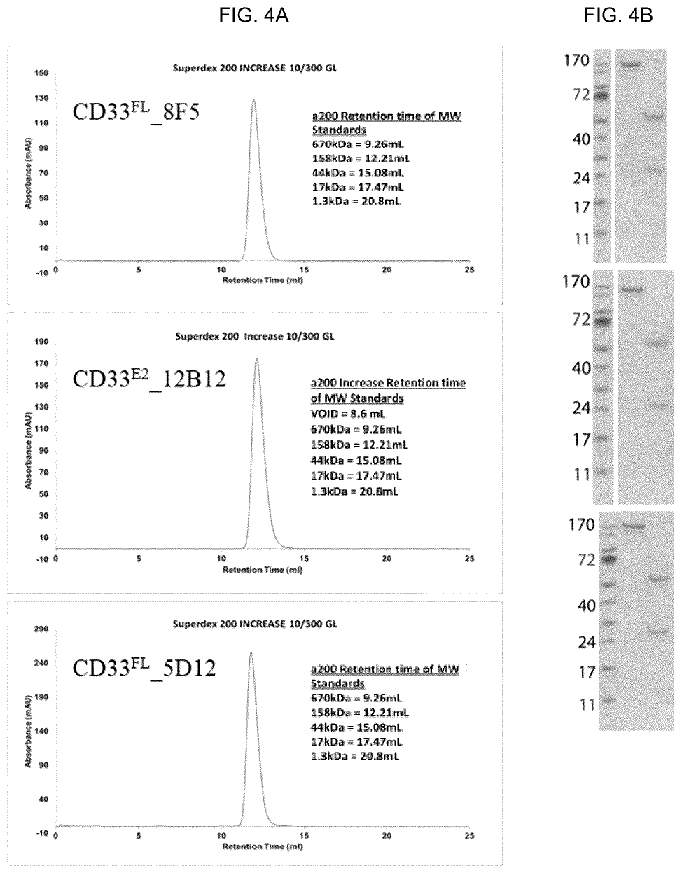

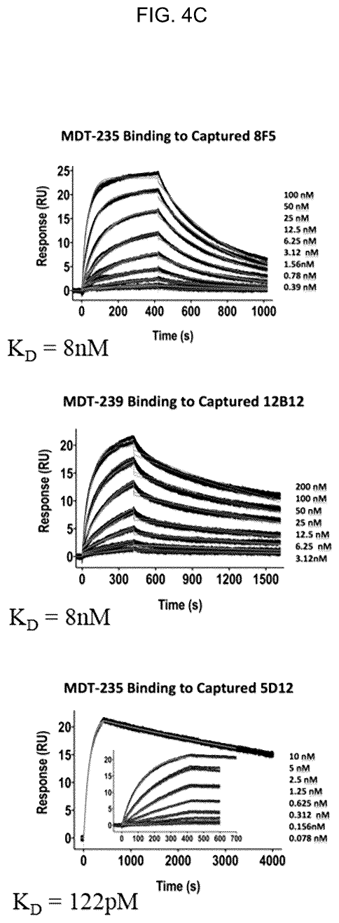

In particular embodiments, “bind” means that the variable region associates with its target epitope with a dissociation constant (Kd or KD) of 10⁻⁸ M or less, in particular embodiments of from 10⁻⁵ M to 10⁻¹³ M, in particular embodiments of from 10⁻⁵ M to 10⁻¹⁰ M, in particular embodiments of from 10⁻⁵ M to 10⁻⁷M, in particular embodiments of from 10⁻⁸ M to 10⁻¹³ M, or in particular embodiments of from 10⁻⁹ M to 10⁻¹³ M. The term can be further used to indicate that the variable region does not bind to other biomolecules present (e.g., it binds to other biomolecules with a dissociation constant (Kd) of 10⁻⁴ M or more, in particular embodiments of from 10⁻⁴ M to 1 M). Binding can also reflect values as depicted in FIG. 4C (K_{D}=8 nM (8F5, 12B12); 122 pM (5D12)).

In particular embodiments, Kd can be characterized using BIAcore. For example, in particular embodiments, Kd can be measured using surface plasmon resonance assays using a BIACORE@-2000 or a BIACORE®-3000 (BIAcore, Inc., Piscataway, N.J.) at 25° C. with immobilized antigen CM5 chips at 10 response units (RU). Briefly, carboxymethylated dextran biosensor chips (CM5, BIACORE, Inc.) can be activated with N-ethyl-N′-(3-dimethylaminopropyl)-carbodiimide hydrochloride (EDC) and N-hydroxysuccinimide (NHS) according to the supplier's instructions. Antigen can be diluted with 10 mM sodium acetate, pH 4.8, to 5 μg/ml (0.2 uM) before injection at a flow rate of 5 ul/minute to achieve y 10 response units (RU) of coupled protein. Following the injection of antigen, 1 M ethanolamine can be injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of 25 ul/min. Association rates (kₒₙ) and dissociation rates (k_{off}) can be calculated using a simple one-to-one Langmuir binding model (BIACORE® Evaluation Software version 3.2) by simultaneously fitting the association and dissociation sensorgrams. The equilibrium dissociation constant (Kd) can be calculated as the ratio k_{off}/kₒn. See, e.g., Chen et al., J. Mol. Biol. 293:865-881, 1999. If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

Unless otherwise indicated, the term “antibody” includes (in addition to antibodies having two full-length heavy chains and two full-length light chains as described above) variants, derivatives, and fragments thereof, examples of which are described below. Furthermore, unless explicitly excluded, antibodies can include monoclonal antibodies, human or humanized antibodies, bispecific antibodies, trispecific antibodies, tetraspecific antibodies, multi-specific antibodies, polyclonal antibodies, linear antibodies, minibodies, domain antibodies, synthetic antibodies, chimeric antibodies, antibody fusions, and fragments thereof, respectively. In particular embodiments, antibodies can include oligomers or multiplexed versions of antibodies.

A monoclonal antibody refers to an antibody obtained from a population of substantially homogeneous antibodies, i.e., the individual antibodies including the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies, and is not to be construed as requiring production of the antibody by any particular method. For example, monoclonal antibodies can be made by a variety of techniques, including the hybridoma method, recombinant DNA methods, phage-display methods, and methods utilizing transgenic animals containing all or part of the human immunoglobulin loci.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al. (supra). In particular embodiments, for the V_{H}, the subgroup is subgroup III as in Kabat et al. (supra).

Human antibodies can be produced using various techniques known in the art. Human antibodies are described generally in van Dijk and van de Winkel, Curr. Opin. Pharmacol. 5:368-74, 2001; and Lonberg, Curr. Opin. Immunol. 20:450-459, 2008. Human antibodies may be prepared by administering an immunogen to a transgenic animal that has been modified to produce intact human antibodies or intact antibodies with human variable regions in response to antigenic challenge. Such animals contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125, 2005. See also, e.g., U.S. Pat. Nos. 6,075,181 and 6,150,584 describing XENOMOUSE™ technology; U.S. Pat. No. 5,770,429 describing HUMAB technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001, 1984; Brodeur et al., Monoclonal Antibody Production Techniques and Applications, pp. 51-63 (Marcel Dekker, Inc., New York, 1987); and Boerner et al., J. Immunol., 147:86, 1991. ) Human antibodies generated via human B-cell hybridoma technology are also described in Li et al., Proc. Natl. Acad. Sci. USA, 103:3557-3562, 2006. Additional methods include those described, for example, in U.S. Pat. No. 7,189,826(describing production of monoclonal human IgM antibodies from hybridoma cell lines) and Ni, Xiandai Mianyixue, 26(4): 265-268, 2006 (describing human-human hybridomas). Human hybridoma technology (Trioma technology) is also described in Vollmers and Brandlein, Histology and Histopathology, 20(3): 927-937, 2005; and Vollmers and Brandlein, Methods and Findings in Experimental and Clinical Pharmacology, 27(3): 185-191, 2005.

A “humanized” antibody refers to a chimeric antibody including amino acid residues from non-human CDRs and amino acid residues from human FRs. In particular embodiments, a humanized antibody will include substantially all of at least one, and typically two, variable domains, in which all or substantially all of the CDRs correspond to those of a non-human antibody, and all or substantially all of the FRs correspond to those of a human antibody. A humanized antibody optionally may include at least a portion of an antibody constant region derived from a human antibody. A “humanized form” of an antibody, e.g., a non-human antibody, refers to an antibody that has undergone humanization.

Humanized antibodies and methods of making them are reviewed, e.g., in Almagro and Fransson, Front. Biosci. 13:1619-1633, 2008, and are further described, e.g., in Riechmann et al., Nature 332:323-329, 1988; Queen et al., Proc. Nat'l Acad. Sci. USA 86:10029-10033, 1989; U.S. Pat. Nos. 5,821,337, 7,527,791, 6,982,321, and 7,087,409; Kashmiri et al., Methods 36:25-34, 2005 (describing SDR (a-CDR) grafting); Padlan, Mol. Immunol. 28:489-498, 1991 (describing “resurfacing”); Dall'Acqua et al., Methods 36:43-60,2005 (describing “FR shuffling”); and Osbourn et al., Methods 36:61-68, 2005 and Klimka et al., Br. J. Cancer, 83:252-260, 2000 (describing the “guided selection” approach to FR shuffling). EP-B-0239400 provides additional description of “CDR-grafting”, in which one or more CDR sequences of a first antibody is/are placed within a framework of sequences not of that antibody, for instance of another antibody.

Human framework regions that may be used for humanization include: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al., Proc. Nati. Acad. Sci. USA, 89:4285, 1992; and Presta et al., J. Immunol., 151:2623, 1993); human mature (somatically mutated) framework regions or human germline framework regions (see, e.g., Almagro and Fransson, Front. Biosci. 13:1619-1633, 2008); and framework regions derived from screening FR libraries (see, e.g., Baca et al., J. Biol. Chem. 272:10678-10684, 1997; and Rosok et al., J. Biol. Chem. 271:22611-22618, 1996).

Referring to the sequences provided herein, the following variable light (V_{L}) and variable heavy (V_{H}) chains, and CDRs, are provided for disclosed antibodies with the indicated specificities:

TABLE 1
Summary of Antibody Sequences

| Antibody Name | Specific For | Chain | SEQ ID NO: |
|---|---|---|---|
| 5D12 | V-set Ig-like domain | V_{L} | 8 |
| | (e.g., in CD33^{FL}) | V_{H} | 9 |
| | | CDRH1 | 94 |
| | | CDRH2 | 95 |
| | | CDRH3 | 96 |
| | | CDRL1 | 97 |
| | | CDRL2 | 98 |
| | | CDRL3 | 99 |
| 8F5 | V-set Ig-like domain | V_{L} | 10 |
| | (e.g., in CD33^{FL}) | V_{H} | 11 |
| | | CDRH1 | 100 |
| | | CDRH2 | 101 |
| | | CDRH3 | 102 |
| | | CDRL1 | 103 |
| | | CDRL2 | 104 |
| | | CDRL3 | 105 |
| 12B12 | C2-set Ig-like domain | V_{L} | 12 |
| | in the absence of V-set | V_{H} | 13 |
| | Ig-like domain (e.g., | CDRH1 | 106 |
| | in CD33^{ΔE2}) | CDRH2 | 107 |
| | | CDRH3 | 108 |
| | | CDRL1 | 109 |
| | | CDRL2 | 110 |
| | | CDRL3 | 111 |
| 4H10 | C2-set Ig-like domain | V_{L} | 14 |
| | in the absence of V-set | V_{H} | 15 |
| | Ig-like domain (e.g., | CDRH1 | 112 |
| | in CD33^{ΔE2}) | CDRH2 | 113 |
| | | CDRH3 | 114 |
| | | CDRL1 | 115 |
| | | CDRL2 | 116 |
| | | CDRL3 | 117 |
| 11D5 | C2-set Ig-like domain | V_{L} | 16 |
| | in the absence of V-set | V_{H} | 17 |
| | Ig-like domain (e.g., | CDRH1 | 118 |
| | in CD33^{ΔE2}) | CDRH2 | 119 |
| | | CDRH3 | 120 |
| | | CDRL1 | 121 |
| | | CDRL2 | 122 |
| | | CDRL3 | 123 |
| 13E11 | C2-set Ig-like domain | V_{L} | 18 |
| | in the absence of V-set | V_{H} | 19 |
| | Ig-like domain (e.g., | CDRH1 | 124 |
| | in CD33^{ΔE2}) | CDRH2 | 125 |
| | | CDRH3 | 126 |
| | | CDRL1 | 127 |
| | | CDRL2 | 128 |
| | | CDRL3 | 129 |
| 5E10.7 | Pan binder, which binds | V_{L} | 20 |
| | C2-set Ig-like domain in | V_{H} | 21 |
| | the presence or absence | CDRH1 | 130 |
| | of V-set Ig-like domain | CDRH2 | 131 |
| | (e.g., in CD33^{FL} | CDRH3 | 132 |
| | and CD33^{ΔE2}) | CDRL1 | 133 |
| | | CDRL2 | 134 |
| | | CDRL3 | 135 |
| 1H7 | Pan binder, which binds | V_{L} | 84 |
| | C2-set Ig-like domain in | V_{H} | 83 |
| | the presence or absence | CDRL1 | 85 |
| | of V-set Ig-like domain | CDRL2 | 86 |
| | (e.g., in CD33^{FL} | CDRL3 | 87 |
| | and CD33^{ΔE2}) | CDRH1 | 88 |
| | | CDRH2 | 89 |
| | | CDRH3 | 90 |
| 11D11 | C2-set Ig-like domain | V_{L} | 22 |
| | in the absence of V-set | V_{H} | 23 |
| | Ig-like domain (e.g., | CDRH1 | 136 |
| | in CD33^{ΔE2}) | CDRH2 | 137 |
| | | CDRH3 | 138 |
| | | CDRL1 | 139 |
| | | CDRL2 | 140 |
| | | CDRL3 | 141 |
| 7E7 | C2-set Ig-like domain | V_{L} | 24 |
| | in the absence of V-set | V_{H} | 25 |
| | Ig-like domain (e.g., | CDRH1 | 142 |
| | in CD33^{ΔE2}) | CDRH2 | 143 |
| | | CDRH3 | 144 |
| | | CDRL1 | 145 |
| | | CDRL2 | 146 |
| | | CDRL3 | 147 |
| 2D5 | Pan binder, which binds | V_{L} | 154 |
| | C2-set Ig-like domain in | V_{H} | 155 |
| | the presence or absence | CDRH1 | 148 |
| | of V-set Ig-like domain | CDRH2 | 149 |
| | (e.g., in CD33^{FL} | CDRH3 | 150 |
| | and CD33^{ΔE2}) | CDRL1 | 151 |
| | | CDRL2 | 152 |
| | | CDRL3 | 153 |

Antibodies disclosed herein can be utilized to prepare various forms of relevant binding domain molecules. For example, particular embodiments can include binding fragments of an antibody, e.g., Fv, Fab, Fab′, F (ab′)₂, Fc, and single chain Fv fragments (scFvs) or any biologically effective fragments of an immunoglobulin that bind specifically to an epitope described herein.

In particular embodiments, an antibody fragment is used. An “antibody fragment” denotes a portion of a complete or full-length antibody that retains the ability to bind to an epitope. Antibody fragments can be made by various techniques, including proteolytic digestion of an intact antibody as well as production by recombinant host-cells (e.g., mammalian suspension cell lines, E. coli or phage), as described herein. Antibody fragments can be screened for their binding properties in the same manner as intact antibodies. Examples of antibody fragments include Fv, scFv, Fab, Fab′, Fab′-SH, F(ab′)₂; diabodies; and linear antibodies.

A single chain variable fragment (scFv) is a fusion protein of the variable regions of the heavy and light chains of immunoglobulins connected with a short linker peptide. Fv fragments include the V_{L} and V_{H} domains of a single arm of an antibody but lack the constant regions. Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). For additional information regarding Fv and scFv, see e.g., Bird, et al., Science 242:423-426, 1988; Huston, et al., Proc. Natl. Acad. Sci. USA 85:5879-5883, 1988; Plueckthun, in The Pharmacology of Monoclonal Antibodies, vol. 113, Rosenburg and Moore (eds.), Springer-Verlag, New York), (1994) 269-315; WO 1993/16185; U.S. Pat. Nos. 5,571,894; and 5,587,458.

A Fab fragment is a monovalent antibody fragment including V_{L}, V_{H}, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90:6444-6448, 1993. Dual affinity retargeting antibodies (DART™; based on the diabody format but featuring a C-terminal disulfide bridge for additional stabilization (Moore et al., Blood 117:4542-51, 2011)) can also be used. Antibody fragments can also include isolated CDRs. For a review of antibody fragments, see Hudson, et al., Nat. Med. 9:129-134, 2003.

In particular embodiments, an antibody disclosed herein can be a bispecific antibody. A bispecific antibody includes an antibody capable of selectively binding two or more epitopes.

Where bispecific antibodies are to be used, these may be conventional bispecific antibodies, which can be manufactured in a variety of ways (Holliger & Winter, Current Opinion Biotechnol. 4, 446-449(1993)), for instance, prepared chemically or from hybrid hybridomas, or may be any of the bispecific antibody fragments mentioned above. scFv dimers or diabodies may be used, rather than whole antibodies. Diabodies and scFv can be constructed without an Fc region, using only variable domains (usually including the variable domain components from both light and heavy chains of the source antibody), potentially reducing the effects of anti-idiotypic reaction. Other forms of bispecific antibodies include the single chain “Janusins” described in Traunecker et al. (Embo Journal, 10, 3655-3659, 1991).

Bispecific antibodies generally include two different binding domains, with each binding domain specifically binding a different epitope either on two different antigens or on the same antigen. If a bispecific antibody is capable of selectively binding two different epitopes (a first epitope and a second epitope), the affinity of the first binding for the first epitope will generally be at least one to two or three or four orders of magnitude lower than the affinity of the first binding domain for the second epitope, and vice versa. The epitopes recognized by the bispecific antibody can be on the same or a different target (e.g., on the same or a different protein). Bispecific antibodies can be made, for example, by combining binding domains that recognize different epitopes of the same antigen.

Some example bispecific antibodies have two heavy chains (each having three heavy chain CDRs, followed by (N-terminal to C-terminal) a CH1 domain, a hinge, a CH2 domain, and a CH3 domain), and two immunoglobulin light chains that confer antigen-binding specificity through association with each heavy chain. However, additional architectures are envisioned, including bi-specific antibodies in which the light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes.

In particular embodiments, a bispecific antibody can include an antibody arm combined with an arm that binds to a triggering molecule on a leukocyte, such as a T-cell receptor molecule (for example, CD3), or Fc receptors for IgG (Fc gamma R), such as Fc gamma RI (CD64), Fc gamma RII (CD32) and Fc gamma RIII (CD 16), so as to focus and localize cellular defense mechanisms to the targeted disease cell. Bispecific antibodies also can be used to localize cytotoxic agents to targeted disease cells.

Bispecific antibodies can be prepared as full-length antibodies or antibody fragments (for example, F(ab′)₂ bispecific antibodies). For example, WO 1996/016673 describes a bispecific anti-ErbB2/anti-Fc gamma RIll antibody; U.S. Pat. No. 5,837,234 describes a bispecific anti-ErbB2/anti-Fc gamma RI antibody; WO 1998/002463 describes a bispecific anti-ErbB2/Fc alpha antibody; U.S. Pat. No. 5,821,337 describes a bispecific anti-ErbB2/anti-CD3 antibody. In particular embodiments, a bispecific antibody can be a bispecific T-cell engaging antibody (developed, for example, by Amgen, Thousand Oaks, CA under the tradename (BiTE®).

In particular embodiments, a bispecific antibody can have an extended half-life. In particular embodiments, half-life extension of a bispecific antibody can be achieved by: increasing the hydrodynamic volume of the antibody by coupling to inert polymers such as polyethylene glycol or other mimetic hydrophilic polymers; fusion or conjugation to large disordered peptides; fusing or coupling the antibody to a ligand, such as an Fc domain or to serum albumin, that binds neonatal Fc receptor (FcRn) directly to take advantage of FcRn-mediated recycling, as used by endogenous substrates such as IgGs and serum albumin; engaging FcRn recycling indirectly by fusion or coupling to a moiety that binds non-covalently to Fc or albumin (e.g., moieties such as IgG-binding domains or albumin-binding moieties, such as organic molecules (AlbuTag), fatty acids (myristic acid), peptides, binding domains from natural sources (Streptococcal protein G) and antibody modular domains (AlbudAbs, nanobodies)). In particular embodiments, the antibody can be fused or coupled to an Fc polypeptide that includes amino acid alterations that extend the in vivo half-life of an antibody that contains the altered Fc polypeptide as compared to the half-life of a similar antibody containing the same Fc polypeptide without the amino acid alterations. Such alterations can be included in an Fc polypeptide that is part of a bispecific antibody described herein. In particular embodiments, Fc polypeptide amino acid alterations can include M252Y, S254T, T256E, M428L, and/or N434S and can be used together, separately or in any combination. These alterations and a number of others are described in U.S. Pat. Nos. 7,083,784, 7,670,600, US Publication No. 2010/0234575, PCT/US2012/070146, and Zwolak, Scientific Reports 7:15521, 2017. In particular embodiments, any substitution at one of the following amino acid positions in an Fc polypeptide can be considered an Fc alteration that extends half-life: 250, 251, 252, 259, 307, 308, 332, 378, 380, 428, 430, 434, 436. Each of these alterations or combinations of these alterations can be used to extend the half-life of a bispecific antibody as described herein. Bispecific antibodies with extended half-lives are described in, for example, U.S. Pat. No. 8,921,528 and US Patent Publication No. 2014/0308285.

Methods for making bispecific antibodies are known in the art. Traditional production of full length bispecific antibodies is based on the co-expression of two immunoglobulin heavy chain-light chain pairs, where the two chains have different specificities (see, for example, Millstein et al. Nature 305:37-39, 1983). Similar procedures are disclosed in, for example, WO 1993/008829, Traunecker et al., EMBO J. 10:3655-3659, 1991.

Techniques for generating bispecific antibodies from antibody fragments also have been described in the literature. For example, bispecific antibodies can be prepared using chemical linkage. For example, Brennan et al. (Science 229:81, 1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated then are converted to thionitrobenzoate (TNB) derivatives. One of the Fab′-TNB derivatives then is reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Binding domains derived from the antibodies disclosed herein can also be utilized within bispecific T-cell engaging antibody constructs. Bispecific T-cell engaging antibody constructs bind both a cancer antigen (e.g., CD33) on tumor cells and an immune cell (e.g., T-cell) activating epitope, with the goal of bringing immune cells to cancer cells to destroy the cancer cells. See, for example, US 2008/0145362. By way of example, SEQ ID NO: 91 is the amino acid sequence of a scFv-based bispecific T-cell engaging antibody construct containing 1H7 (based on SEQ ID NOs: 83 & 84) and a CD3 binding domain; SEQ ID NOs: 92 and 93 are the amino acid sequences of the light chain and heavy chain (respectively; based on SEQ ID NO: 83 and 84) of an IgG-based bispecific T-cell engaging antibody construct containing a CD3 binding domain and 1H7. See Example 7.

In particular embodiments, binding domains derived from the CD33 monoclonal antibodies disclosed herein can be coupled with an immune cell activating epitope to form a bispecific T-cell engaging antibody construct. Immune cells that can be targeted for localized activation by bispecific T-cell engaging antibody constructs of the current disclosure include, for example, T-cells, natural killer (NK) cells, and macrophages.

T-cell activation can be mediated by two distinct signals: those that initiate antigen-dependent primary activation and provide a T-cell receptor like signal (primary cytoplasmic signaling sequences) and those that act in an antigen-independent manner to provide a secondary or co-stimulatory signal (secondary cytoplasmic signaling sequences). Bispecific T-cell engaging antibody constructs disclosed herein can target any T-cell activating epitope that upon binding induces T-cell activation. Examples of such T-cell activating epitopes are on T-cell markers including CD2, CD3, CD7, CD27, CD28, CD30, CD40, CD83, 4-1BB (CD 137), OX40, lymphocyte function-associated antigen-1 (LFA-1), LIGHT, NKG2C, and B7-H3.

CD3 is a primary signal transduction element of T-cell receptors. CD3 is composed of a group of invariant proteins called gamma (γ), delta (Δ), epsilon (Σ), zeta (Z) and eta (H) chains. The γ, Δ, and Σ chains are structurally-related, each containing an Ig-like extracellular constant domain followed by a transmembrane region and a cytoplasmic domain of more than 40 amino acids. The Z and H chains have a distinctly different structure: both have a very short extracellular region of only 9 amino acids, a transmembrane region and a long cytoplasmic tail including 113 and 115 amino acids in the Z and H chains, respectively. The invariant protein chains in the CD3 complex associate to form noncovalent heterodimers of the Σ chain with a γ chain (Σγ) or with a Δ chain (ΣΔ) or of the Z and H chain (ZH), or a disulfide-linked homodimer of two Z chains (ZZ). 90% of the CD3 complex incorporate the ZZ homodimer.

The cytoplasmic regions of the CD3 chains include a motif designated the immunoreceptor tyrosine-based activation motif (ITAM). This motif is found in a number of other receptors including the Ig-α/Ig-β heterodimer of the B-cell receptor complex and Fc receptors for IgE and IgG. The ITAM sites associate with cytoplasmic tyrosine kinases and participate in signal transduction following TCR-mediated triggering. In CD3, the γ, Δ and Σ chains each contain a single copy of ITAM, whereas the Z and H chains harbor three ITAMs in their long cytoplasmic regions. Indeed, the Z and H chains have been ascribed a major role in T-cell activation signal transduction pathways.

In particular embodiments, the CD3 binding domain (e.g., scFv) is derived from the OKT3 antibody (the same as the one utilized in blinatumomab). The OKT3 antibody is described in detail in U.S. Pat. No. 5,929,212. It includes a variable light chain including a CDRL1 sequence including SASSSVSYMN (SEQ ID NO: 38), a CDRL2 sequence including RWIYDTSKLAS (SEQ ID NO: 39), and a CDRL3 sequence including QQWSSNPFT (SEQ ID NO: 40). In particular embodiments, the CD3 T-cell activating epitope binding domain is a human or humanized binding domain (e.g., scFv) including a variable heavy chain including a CDRH1 sequence including KASGYTFTRYTMH (SEQ ID NO: 41), a CDRH2 sequence including INPSRGYTNYNQKFKD (SEQ ID NO: 42), and a CDRH3 sequence including YYDDHYCLDY (SEQ ID NO: 43).

The following sequence is an scFv derived from OKT3 which retains the capacity to bind CD3: QVQLQQSGAELARPGASVKMSCKASGYTFTRYTMHWVKQRPGQGLEWIGYINPSRGYTNYN QKFKDKATLTTDKSSSTAYMQLSSLTSEDSAVYYCARYYDDHYCLDYWGQGTTLTVSSSGGG GSGGGGSGGGGSQIVLTQSPAIMSASPGEKVTMTCSASSSVSYMNWYQQKSGTSPKRWIYD TSKLASGVPAHFRGSGSGTSYSLTISGMEAEDAATYYCQQWSSNPFTFGSGTKLEINR (SEQ ID NO: 44). It may also be used as a CD3 binding domain.

In particular embodiments, the CD3 T-cell activating epitope binding domain is a human or humanized binding domain (e.g., scFv) including a variable light chain including a CDRL1 sequence including QSLVHNNGNTY (SEQ ID NO: 45), a CDRL2 sequence including KVS (SEQ ID NO: 46; not included in Sequence Listing), and a CDRL3 sequence including GQGTQYPFT (SEQ ID NO: 47). In particular embodiments, the CD3 T-cell activating epitope binding domain is a human or humanized binding domain (e.g., scFv) including a variable heavy chain including a CDRH1 sequence including GFTFTKAW (SEQ ID NO: 48), a CDRH2 sequence including IKDKSNSYAT (SEQ ID NO: 49), and a CDRH3 sequence including RGVYYALSPFDY (SEQ ID NO: 50). These reflect CDR sequences of the 20G6-F3 antibody.

In particular embodiments, the CD3 T-cell activating epitope binding domain is a human or humanized binding domain (e.g., scFv) including a variable light chain including a CDRL1 sequence including QSLVHDNGNTY (SEQ ID NO: 51), a CDRL2 sequence including KVS (SEQ ID NO: 52; not included in Sequence Listing), and a CDRL3 sequence including GQGTQYPFT (SEQ ID NO: 53). In particular embodiments, the CD3 T-cell activating epitope binding domain is a human or humanized binding domain (e.g., scFv) including a variable heavy chain including a CDRH1 sequence including GFTFSNAW (SEQ ID NO: 54), a CDRH2 sequence including IKARSNNYAT (SEQ ID NO: 55), and a CDRH3 sequence including RGTYYASKPFDY (SEQ ID NO: 56). These reflect CDR sequences of the 4B4-D7 antibody.

In particular embodiments, the CD3 T-cell activating epitope binding domain is a human or humanized binding domain (e.g., scFv) including a variable light chain including a CDRL1 sequence including QSLEHNNGNTY (SEQ ID NO: 57), a CDRL2 sequence including KVS (SEQ ID NO: 58; not included in Sequence Listing), and a CDRL3 sequence including GQGTQYPFT (SEQ ID NO: 59). In particular embodiments, the CD3 T-cell activating epitope binding domain is a human or humanized binding domain (e.g., scFv) including a variable heavy chain including a CDRH1 sequence including GFTFSNAW (SEQ ID NO: 60), a CDRH2 sequence including IKDKSNNYAT (SEQ ID NO: 61), and a CDRH3 sequence including RYVHYGIGYAMDA (SEQ ID NO: 62). These reflect CDR sequences of the 4E7-C9 antibody.

In particular embodiments, the CD3 T-cell activating epitope binding domain is a human or humanized binding domain (e.g., scFv) including a variable light chain including a CDRL1 sequence including QSLVHTNGNTY (SEQ ID NO: 63), a CDRL2 sequence including KVS (SEQ ID NO: 64; not included in Sequence Listing), and a CDRL3 sequence including GQGTHYPFT (SEQ ID NO: 65). In particular embodiments, the CD3 T-cell activating epitope binding domain is a human or humanized binding domain (e.g., scFv) including a variable heavy chain including a CDRH1 sequence including GFTFTNAW (SEQ ID NO: 66), a CDRH2 sequence including KDKSNNYAT (SEQ ID NO: 67), and a CDRH3 sequence including RYVHYRFAYALDA (SEQ ID NO: 68). These reflect CDR sequences of the 18F5-H10 antibody.

Additional examples of anti-CD3 antibodies, binding domains, and CDRs can be found in WO2016/116626. TR66 may also be used.

CD28 is a surface glycoprotein present on 80% of peripheral T-cells in humans, and is present on both resting and activated T-cells. CD28 binds to B7-1 (CD80) and B7-2 (CD86) and is the most potent of the known co-stimulatory molecules (June et al., Immunol. Today 15:321, 1994; Linsley et al., Ann. Rev. Immunol. 11:191, 1993). In particular embodiments, the CD28 binding domain (e.g., scFv) is derived from CD80, CD86 or the 9D7 antibody. Additional antibodies that bind CD28 include 9.3, KOLT-2, 15E8, 248.23.2, and EX5.3D10. Further, 1YJD provides a crystal structure of human CD28 in complex with the Fab fragment of a mitogenic antibody (5.11A1). In particular embodiments, antibodies that do not compete with 9D7 are selected.

Particular embodiments disclosed herein including binding domains that bind epitopes on CD8. In particular embodiments, the CD8 binding domain (e.g., scFv) is derived from the OKT8 antibody. For example, in particular embodiments, the CD8 T-cell activating epitope binding domain is a human or humanized binding domain (e.g., scFv) including a variable light chain including a CDRL1 sequence including RTSRSISQYLA (SEQ ID NO: 69), a CDRL2 sequence including SGSTLQS (SEQ ID NO: 70), and a CDRL3 sequence including QQHNENPLT (SEQ ID NO: 71). In particular embodiments, the CD8 T-cell activating epitope binding domain is a human or humanized binding domain (e.g., scFv) including a variable heavy chain including a CDRH1 sequence including GFNIKD (SEQ ID NO: 72), a CDRH2 sequence including RIDPANDNT (SEQ ID NO: 73), and a CDRH3 sequence including GYGYYVFDH (SEQ ID NO: 74). These reflect CDR sequences of the OKT8 antibody.

In particular embodiments, a binding domain is a single chain T-cell receptor (scTCR) including V_{α/β} and C_{α/β} chains (e.g., V_{α}-C_{α}, V_{β}-C_{β}, V_{α}-V_{β}) or including V_{α}-C_{α}, V_{β}-C_{β}, V_{α}- V_{β} pair specific for a target epitope of interest. In particular embodiments, T-cell activating epitope binding domains can be derived from or based on a V_{α}, V_{β}, C_{α}, or C_{β} of a known TCR (e.g., a high-affinity TCR).

In particular embodiments natural killer cells (also known as NK-cells, K-cells, and killer cells) are targeted for localized activation by bispecific T-cell engaging antibody constructs. NK cells can induce apoptosis or cell lysis by releasing granules that disrupt cellular membranes, and can secrete cytokines to recruit other immune cells.

Examples of activating proteins expressed on the surface of NK cells include NKG2D, CD8, CD16, KIR2DL4, KIR2DS1, KIR2DS2, KIR3DS1, NKG2C, NKG2E, NKG2D, and several members of the natural cytotoxicity receptor (NCR) family. Examples of NCRs that activate NK cells upon ligand binding include NKp30, NKp44, NKp46, NKp80, and DNAM-1.

Examples of commercially available antibodies that bind to an NK cell receptor and induce and/or enhance activation of NK cells include: 5C6 and 1D11, which bind and activate NKG2D (available from BioLegend® San Diego, CA); mAb 33, which binds and activates KIR2DL4 (available from BioLegend®); P44-8, which binds and activates NKp44 (available from BioLegend®); SK1, which binds and activates CD8; and 3G8 which binds and activates CD16.

In particular embodiments, the bispecific T-cell engaging antibody construct can bind to and block an NK cell inhibitory receptor to enhance NK cell activation. Examples of NK cell inhibitory receptors that can be bound and blocked include KIR2DL1, KIR2DL2/3, KIR3DL1, NKG2A, and KLRG1. In particular embodiments, a binding domain that binds and blocks the NK cell inhibitory receptors KIR2DL1 and KIR2DL2/3 includes a variable light chain region of the sequence EIVLTQSPVTLSLSPGERATLSCRASQSVSSYLAWYQQKPGQAPRLLIYDASNRATGIPARFSG SGSGTDFTLTISSLEPEDFAVYYCQQRSNWMYTFGQGTKLEIKRT (SEQ ID NO: 75) and a variable heavy chain region of the sequence QVQLVQSGAEVKKPGSSVKVS CKASGGTFSFYAISWVRQAPGQGLEWMGGFIPIFGAANYAQKFQGRVTITADESTSTAYMELS SLRSDDTAVYYCARIPSGSYYYDYDMDVWGQGTTVTVSS (SEQ ID NO: 76). Additional NK cell activating antibodies are described in WO/2005/0003172 and U.S. Pat. No. 9,415,104.

In particular embodiments macrophages are targeted for localized activation by bispecific T-cell engaging antibody constructs. Macrophages are a type of leukocyte (or white blood cell) that can engulf and digest cells, cellular debris, and/or foreign substances in a process known as phagocytosis.

The bispecific T-cell engaging antibody constructs can be designed to bind to a protein expressed on the surface of macrophages. Examples of activating proteins expressed on the surface of macrophages (and their precursors, monocytes) include CD11b, CD11c, CD64, CD68, CD119, CD163, CD206, CD209, F4/80, IFGR2 Toll-like receptors (TLRs) 1-9, IL-4Rα, and MARCO. Commercially available antibodies that bind to proteins expressed on the surface of macrophages include M1/70, which binds and activates CD11b (available from BioLegend®); KP1, which binds and activates CD68 (available from ABCAM®, Cambridge, United Kingdom); and ab87099, which binds and activates CD163 (available from ABCAM®).

In particular embodiments, bispecific T-cell engaging antibody constructs can target a pathogen recognition receptor (PRR). PRRs are proteins or protein complexes that recognize a danger signal and activate and/or enhance the innate immune response. Examples of PRRs include the TLR4/MD-2 complex, which recognizes gram negative bacteria; Dectin-1 and Dectin-2, which recognize mannose moieties on fungus and other pathogens; TLR2/TLR6 or TLR2/TLR1 heterodimers, which recognize gram positive bacteria; TLR5, which recognizes flagellin; and TLR9 (CD289), which recognizes CpG motifs in DNA. In particular embodiments, bispecific T-cell engaging antibody constructs can bind and activate TLR4/MD-2, Dectin-1, Dectin-2, TRL2/TLR6, TLR2/TLR1, TLR5, and/or TLR9.

In particular embodiments, bispecific T-cell engaging antibody constructs can target the complement system. The complement system refers to an immune pathway that is induced by antigen-bound antibodies and involves signaling of complement proteins, resulting in immune recognition and clearance of the antibody-coated antigens.

Binding domains of bispecific T-cell engaging antibody constructs may be joined through a linker. A linker is an amino acid sequence which can provide flexibility and room for conformational movement between the binding domains of a bispecific T-cell engaging antibody construct. Any appropriate linker may be used. Examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target. Commonly used flexible linkers include Gly-Ser linkers such as GGSGGGSGGSG (SEQ ID NO: 77), GGSGGGSGSG (SEQ ID NO: 78) and GGSGGGSG (SEQ ID NO: 79). Additional examples include: GGGGSGGGGS (SEQ ID NO: 80); GGGSGGGS (SEQ ID NO: 81); and GGSGGS (SEQ ID NO: 82). Linkers that include one or more antibody hinge regions and/or immunoglobulin heavy chain constant regions, such as CH3 alone or a CH2CH3 sequence can also be used.

In some situations, flexible linkers may be incapable of maintaining a distance or positioning of binding domains needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

In particular embodiments, bispecific T-cell engaging antibody construct molecules from C2-set Ig-like domain-specific antibodies recognizing C2-set Ig-like domain in the absence of V-set Ig-like domain (e.g., in CD33^{ΔE2}) or C2-set Ig-like domain in the presence or absence of V-set Ig-like domain (e.g., in CD33^{FL} and CD33^{ΔE2}; a pan-binding antibody) can be generated. In particular embodiments, bispecific T-cell engaging antibody construct molecules from V-set Ig-like domain-specific antibodies recognizing the V-set Ig-like domain (e.g., in CD33^{FL}) can be generated. Cytolytic properties of bispecific T-cell engaging antibody constructs can be confirmed in comparative in vitro assays. Briefly, for cell line experiments, target cancer cells can be incubated in 96-well round bottom plates at 5-10,000 cells/well containing increasing concentrations of the various bispecific T-cell engaging antibody constructs (e.g., CD33/CD3 bispecific T-cell engaging antibody construct including AMG 330) with/without healthy donor T-cells (used at an E:T cell ratio of 1:1 and 3:1). After 48 hours, cell numbers and drug-induced cytotoxicity, using 4′,6-diamidino-2-phenylindole (DAPI) to detect non-viable cells, can be determined by flow cytometry. In experiments where healthy donor T-cells are added, cancer cells can be identified by forward/side scatter properties and negativity for CellVue Burgundy dye. Experiments can include technical duplicates.

In particular embodiments, an antibody described herein can be included as part of a chimeric antigen receptor (CAR). “Chimeric antigen receptors” or “CARs” refer to synthetically designed receptors including at least a binding domain (for instance, including domains from both a light and heavy chain) and an effector domain, and optionally a spacer domain and/or a transmembrane domain. In particular embodiments, a CAR refers to a recombinant polypeptide including an extracellular antigen binding domain in the form of a scFv, a transmembrane domain, and cytoplasmic signaling domains (also referred to herein as “an intracellular signaling domains”) including a functional signaling domain derived from a stimulatory molecule as defined below. In particular embodiments, a central intracellular signaling domain of a CAR is derived from the CD3 zeta chain that is normally found associated with the TCR complex. As described more fully below, the CD3 zeta signaling domain can be fused with one or more functional signaling domains derived from at least one co-stimulatory molecule such as 4-1BB (i.e., CD137), CD27 and/or CD28.

Binding domains of CARs of the present disclosure. In particular embodiments, the binding domain binds CD33. Sources of binding domains include antibody variable regions from various species (which can be in the form of antibodies, sFvs, scFvs, Fabs, scFv-based grababody, or soluble V_{H} domain or domain antibodies). Specific CD33 binding domains are described herein, including those which bind specifically only to the C2-set domain or only to the V-set domain of CD33. Specific CD33 binding domains include sequences from the antibodies (or V_{L} or V_{H}, or CDRs) shown in Table 1. These antibodies can form antigen-binding regions using both a heavy and light chain variable region (for instance, functionally linked to form a single-chain antibody molecule), or in some instances only a light or only a heavy chain variable region. If only a heavy chain is used, these functional antibodies are homodimers referred to as “heavy chain antibodies” (Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008).

An alternative source of binding domains includes sequences that encode random peptide libraries or sequences that encode an engineered diversity of amino acids in loop regions of alternative non-antibody scaffolds, such as scTCR (see, e.g., Lake et al., Int. Immunol. 11:745, 1999; Maynard et al., J. Immunol. Methods 306:51, 2005; U.S. Pat. No. 8,361,794), fibrinogen domains (see, e.g., Weisel et al., Science 230:1388, 1985), Kunitz domains (see, e.g., U.S. Pat. No. 6,423,498), designed ankyrin repeat proteins (DARPins) (Binz et al., J. Mol. Biol. 332:489, 2003 and Binz et al., Nat. Biotechnol. 22:575, 2004), fibronectin binding domains (adnectins or monobodies) (Richards et al., J. Mol. Biol. 326:1475, 2003; Parker et al., Protein Eng. Des. Selec. 18:435, 2005 and Hackel et al. (2008) J. Mol. Biol. 381:1238-1252), cysteine-knot miniproteins (Vita et al. (1995) Proc. Nat'l. Acad. Sci. (USA) 92:6404-6408; Martin et al. (2002) Nat. Biotechnol. 21:71, 2002 and Huang et al. (2005) Structure 13:755, 2005), tetratricopeptide repeat domains (Main et al., Structure 11:497, 2003 and Cortajarena et al., ACS Chem. Biol. 3:161, 2008), leucine-rich repeat domains (Stumpp et al., J. Mol. Biol. 332:471, 2003), lipocalin domains (see, e.g., WO 2006/095164, Beste et al., Proc. Nat'l. Acad. Sci. (USA) 96:1898, 1999 and Schönfeld et al., Proc. Nat'l. Acad. Sci. (USA) 106:8198, 2009), V-like domains (see, e.g., US Patent Application Publication No. 2007/0065431), C-type lectin domains (Zelensky and Gready, FEBS J. 272:6179, 2005; Beavil et al., Proc. Nat'l. Acad. Sci. (USA) 89:753, 1992 and Sato et al., Proc. Nat'l. Acad. Sci. (USA) 100:7779, 2003), mAb2 or Fcab (Fc antigen binding) (see, e.g., PCT Patent Application Publication Nos. WO 2007/098934 and WO 2006/072620; Wozniak-Knopp et al., Prot. Eng. Des. Select. 23:4, 289-297, 2010), armadillo repeat proteins (see, e.g., Madhurantakam et al., Protein Sci. 21:1015, 2012; PCT Patent Application Publication No. WO 2009/040338), affilin (Ebersbach et al., J. Mol. Biol. 372:172, 2007), affibody, avimers, knottins, fynomers, atrimers, cytotoxic T-lymphocyte associated protein-4 (Weidle et al., Cancer Gen. Proteo. 10:155, 2013) or the like (Nord et al., Protein Eng. 8:601, 1995; Nord et al., Nat. Biotechnol. 15:772, 1997; Nord et al., Euro. J. Biochem. 268:4269, 2001; Binz et al., Nat. Biotechnol. 23:1257, 2005; Boersma and Plückthun, Curr. Opin. Biotechnol. 22:849, 2011).

Effector Domains of CARs of the present disclosure. Effector domains are capable of transmitting functional signals to a cell. In particular embodiments, an effector domain will directly or indirectly promote a cellular response by associating with one or more other proteins that directly promote a cellular response. Effector domains can provide for activation of at least one function of a transduced lymphocyte expressing the CAR upon binding to CD33 on a targeted cell. Activation of the lymphocyte can include one or more of proliferation, differentiation, activation or other effector functions.

An effector domain may include one, two, three or more receptor signaling domains, intracellular signaling domains, costimulatory domains, or combinations thereof. Any intracellular effector domain, costimulatory domain or both from any of a variety of signaling molecules (e.g., signal transduction receptors) may be used in the CARs of this disclosure.

Exemplary effector domains include those from 4-1BB, CD38, CD30, CD3Z, CD27, CD28, CD79A, CD79B, CARD11, DAP10, FcRa, FcRB, FcRy, Fyn, HVEM, ICOS, Lck, LAG3, LAT, LRP, NOTCH1, Wnt, NKG2D, OX40, ROR2, Ryk, SLAMF1, Slp76, pTa, TCRa, TCRB, TRIM, Zap70, PTCH2, or any combination thereof.

T-cell activation can be said to be mediated by two distinct classes of cytoplasmic signaling sequence: those that initiate antigen-dependent primary activation and provide a T-cell receptor like signal (primary cytoplasmic signaling sequences) and those that act in an antigen-independent manner to provide a secondary or co-stimulatory signal (secondary cytoplasmic signaling sequences). Primary cytoplasmic signaling sequences that act in a stimulatory manner may contain signaling motifs which are known as immunoreceptor tyrosine-based activation motifs or ITAMs. Examples of ITAMs containing primary cytoplasmic signaling sequences include those derived from CD3 zeta, FcR gamma, CD3 gamma, CD3 delta, CD3 epsilon, CD5, CD22, CD79a, CD79b, and CD66d.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a costimulatory factor, or any combination thereof.

Examples of intracellular signaling domains include the cytoplasmic sequences of the CD3zeta chain, and/or co-receptors that act in concert to initiate signal transduction following CAR engagement, as well as any derivative or variant of these sequences and any synthetic sequence that has the same functional capability. In particular embodiments, an intracellular signaling domain of a CAR can be designed to include an intracellular signaling domain combined with any other desired cytoplasmic domain(s). For example, the intracellular signaling domain of a CAR can include an intracellular signaling domain and a costimulatory signaling region. The costimulatory signaling region refers to a portion of the CAR including the intracellular domain of a costimulatory molecule. A costimulatory molecule is a cell surface molecule other than the expressed marker ligand that is required for a response of lymphocytes to a marker. Examples of such molecules include CD27, CD28, 4-1BB (CD 137), OX40, CD30, CD40, lymphocyte function-associated antigen-1 (LFA-1), CD2, CD7, LIGHT, NKG2C, B7-H3, and a ligand that specifically binds with CD83.

CARs disclosed herein can also include spacer region(s). Spacer regions can be customized for individual markers on targets to optimize target recognition. In particular embodiments, a spacer length can be selected based upon the location of a marker epitope, affinity of an antibody for the epitope, and/or the ability of the lymphocytes expressing the CAR to proliferate in vitro and/or in vivo in response to marker recognition.

Typically, a spacer region is found between the binding domain and a transmembrane domain of the CAR. Spacer regions can provide for flexibility of the binding domain and allows for high expression levels in the modified cells. In particular embodiments, a spacer region can have at least 10 to 250 amino acids, at least 10 to 200 amino acids, at least 10 to 150 amino acids, at least 10 to 100 amino acids, at least 10 to 50 amino acids or at least 10 to 25 amino acids and including any integer between the endpoints of any of the listed ranges. particular embodiments, a spacer region has 250 amino acids or less; 200 amino acids or less, 150 amino acids or less; 100 amino acids or less; 50 amino acids or less; 40 amino acids or less; 30 amino acids or less; 20 amino acids or less; or 10 amino acids or less.

In particular embodiments, spacer regions can be derived from a hinge region of an immunoglobulin like molecule, for example all or a portion of the hinge region from a human IgG1, human IgG2, a human IgG3, or a human IgG4. Hinge regions can be modified to avoid undesirable structural interactions such as dimerization. In particular embodiments, all or a portion of a hinge region can be combined with one or more domains of a constant region of an immunoglobulin. For example, a portion of a hinge region can be combined with all or a portion of a CH2 or CH3 domain or variant thereof.

CARs disclosed herein can also include transmembrane domains. The transmembrane domain provides for anchoring of the CAR in the lymphocyte membrane. The transmembrane domain may be derived either from a natural or a synthetic source. When the source is natural, the domain may be derived from any membrane-bound or transmembrane protein. Transmembrane regions include at least the transmembrane region(s) of) the alpha, beta, or zeta chain of the T-cell receptor, CD28, CD3, CD45, CD4, CD8, CD9, CD16, CD22; CD33, CD37, CD64, CD80, CD86, CD134, CD137, and CD154. In particular embodiments, synthetic or variant transmembrane domains include predominantly hydrophobic residues such as leucine and valine.

Different potential CAR nucleic acids that encode different CD33 binding domains, different spacer region lengths, different intracellular binding domains and/or different transmembrane domains, can be tested in vivo (for instance, in an animal model) and/or in vitro to identify CAR(s) with improved function over other CARs.

Variants of antibodies described herein are also included. Variants of antibodies can include those having one or more conservative amino acid substitutions or one or more non-conservative substitutions that do not adversely affect the binding of the protein as indicated in the accompanying FIGs.

In particular embodiments, a conservative amino acid substitution may not substantially change the structural characteristics of the reference sequence (e.g., a replacement amino acid should not tend to break a helix that occurs in the reference sequence, or disrupt other types of secondary structure that characterizes the reference sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden & J. Tooze, eds., Garland Publishing, New York, N.Y. (1991)); and Thornton et al., Nature, 354:105(1991).

In particular embodiments, a “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gln); Group 4: Arginine (Arg), Lysine (Lys), Histidine (His); Group 5: Isoleucine (lle), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and lle. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, lle, Val, and Cys; and large aromatic residues: Phe, Tyr, and Trp. Additional information is found in Creighton (1984) Proteins, W.H. Freeman and Company.

In particular embodiments, a V_{L} region can be derived from or based on a disclosed VL and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the disclosed VL. An insertion, deletion or substitution may be anywhere in the VL region, including at the amino-or carboxy-terminus or both ends of this region, provided that each CDR includes zero changes or at most one, two, or three changes and provided an antibody including the modified V_{L} region can still specifically bind its target epitope with an affinity similar to the wild type binding domain.

In particular embodiments, a V_{H} region can be derived from or based on a disclosed V_{H} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V_{H} disclosed herein. An insertion, deletion or substitution may be anywhere in the V_{H} region, including at the amino-or carboxy-terminus or both ends of this region, provided that each CDR includes zero changes or at most one, two, or three changes and provided an antibody including the modified V_{H} region can still specifically bind its target epitope with an affinity similar to the wild type binding domain.

In particular embodiments including bispecific T-cell engaging antibody constructs, T-cell activating epitope binding domains include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V₆₀ , V_{β}, C₆₀ , or C₆₀ of a known TCR. An insertion, deletion or substitution may be anywhere in a V₆₀ , V_{β}, C₆₀ , or C₆₀ region, including at the amino-or carboxy-terminus or both ends of these regions, provided that each CDR includes zero changes or at most one, two, or three changes and provided a binding domain including a modified V₆₀ , V_{β}, C₆₀ , or C₆₀ region can still specifically bind its target with an affinity similar to wild type.

In particular embodiments, one or more amino acid modifications may be introduced into the Fc region of an antibody, thereby generating an Fc region variant. The Fc region variant may include a human Fc region sequence (e.g., a human IgG1, IgG2, IgG3 or IgG4 Fc region) including an amino acid modification (e.g., a substitution) at one or more amino acid positions.

In particular embodiments, variants have been modified from a reference sequence to produce an administration benefit. Exemplary administration benefits can include (1) reduced susceptibility to proteolysis, (2) reduced susceptibility to oxidation, (3) altered binding affinity for forming protein complexes, (4) altered binding affinities, (5) reduced immunogenicity; and/or (6) extended half-live.

In particular embodiments the antibodies can be mutated to increase the half-life of the antibodies in serum. M428L/N434S is a pair of mutations that increase the half-life of antibodies in serum, as described in Zalevsky et al., Nature Biotechnology 28, 157-159, 2010.

In particular embodiments the antibodies can be mutated to increase their affinity for Fc receptors. Exemplary mutations that increase the affinity for Fc receptors include: G236A/S239D/A330L/1332E (GASDALIE). Smith et al., Proceedings of the National Academy of Sciences of the United States of America, 109(16), 6181-6186, 2012. In particular embodiments, an antibody variant includes an Fc region with one or more amino acid substitutions which improve ADCC, e.g., substitutions at positions 298, 333, and/or 334 of the Fc region (EU numbering of residues). In particular embodiments, alterations are made in the Fc region that result in altered C1q binding and/or Complement Dependent Cytotoxicity (CDC), e.g., as described in U.S. Pat. No. 6,194,551, WO 99/51642, and Idusogie et al., J. Immunol. 164:4178-4184, 2000.

In particular embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further below. In particular embodiments, residue 5400 (EU numbering) of the heavy chain Fc region is selected. Cysteine engineered antibodies may be generated as described, e.g., in U.S. Pat. No. 7,521,541.

In particular embodiments, a variant includes or is a sequence that has at least 90%, at least 91%, at least 92%, at least 93%, at least 94%, at least 95%, at least 96%, at least 97%, at least 98%, at least 99%, at least 99.5% sequence identity to an antibody sequence disclosed herein. In particular embodiments, a variant includes or is a sequence that has at least 90%, at least 91%, at least 92%, at least 93%, at least 94%, at least 95%, at least 96%, at least 97%, at least 98%, at least 99%, at least 99.5% sequence identity to a light chain variable region (V_{L}) and/or to a heavy chain variable region (V_{H}), or both, wherein each CDR includes zero changes or at most one, two, or three changes, from the reference antibody disclosed herein or fragment or derivative thereof that specifically binds to a targeted CD33 epitope.

Antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at position 297 in the Fc region (Eu numbering of Fc region residues); however, Asn297 may also be located +3 amino acids upstream or downstream of position 297, i.e., between positions 294 and 300, due to minor sequence variations in antibodies. Such fucosylation variants may have improved ADCC function. See, e.g., WO2000/61739; WO 2001/29246; WO2002/031140; US2002/0164328; WO2003/085119; WO2003/084570; US2003/0115614; US2003/0157108; US2004/0093621; US2004/0110704; US2004/0132140; US2004/0110282; US2004/0109865; WO2005/035586; WO2005/035778; WO2005/053742; Okazaki et al. J. Mol. Biol. 336:1239-1249(2004); and Yamane-Ohnuki et al. Biotech. Bioeng. 87:614(2004). Examples of cell lines capable of producing defucosylated antibodies include Lec13 CHO cells deficient in protein fucosylation (Ripka et al. Arch. Biochem. Biophys. 249:533-545, 1986, and knockout cell lines, such as alpha-1,6-fucosyltransferase gene, FUT8, knockout CHO cells (see, e.g., Yamane-Ohnuki et al., Biotech. Bioeng. 87:614, 2004; Kanda et al., Biotechnol. Bioeng., 94(4): 680-688, 2006; and WO2003/085107).

In particular embodiments, modified antibodies include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications also include nitrited constructs.

In particular embodiments, variants include glycosylation variants wherein the number and/or type of glycosylation site has been altered compared to the amino acid sequences of a reference sequence. In particular embodiments, glycosylation variants include a greater or a lesser number of N-linked glycosylation sites than the reference sequence. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (e.g., those that are naturally occurring) are eliminated and one or more new N-linked sites are created. Additional antibody variants include cysteine variants wherein one or more cysteine residues are deleted from or substituted for another amino acid (e.g., serine) as compared to the reference sequence. These cysteine variants can be useful when antibodies must be refolded into a biologically active conformation such as after the isolation of insoluble inclusion bodies. These cysteine variants generally have fewer cysteine residues than the reference sequence, and typically have an even number to minimize interactions resulting from unpaired cysteines.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, A. et al., Cancer Biochem. Biophys., 1984, 7:175-186; Hershfield, M. S. et al., N. Engl. J. Medicine, 1987, 316:589-596; and Meyers, F. J. et al., Clin. Pharmacol. Ther., 49:307-313, 1991). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta 788:248, 1984), alter protein immunogenicity (Abuchowski et al., J. Biol. Chem. 252:3582, 1977), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

In particular embodiments, antibodies disclosed herein are formed using the Daedalus expression system as described in Pechman et al. (Am J Physiol 294: R1234-R1239, 2008). The Daedalus system utilizes inclusion of minimized ubiquitous chromatin opening elements in transduction vectors to reduce or prevent genomic silencing and to help maintain the stability of decigram levels of expression. This system can bypass tedious and time-consuming steps of other protein production methods by employing the secretion pathway of serum-free adapted human suspension cell lines, such as 293 Freestyle. Using optimized lentiviral vectors, yields of 20-100 mg/l of correctly folded and post-translationally modified, endotoxin-free protein of up to 70 kDa in size, can be achieved in conventional, small-scale (100 ml) culture. At these yields, most proteins can be purified using a single size-exclusion chromatography step, immediately appropriate for use in structural, biophysical or therapeutic applications. Bandaranayake et al., Nucleic Acids Res., 39(21) 2011. In some instances, purification by chromatography may not be needed due to the purity of manufacture according to the methods described herein.

In particular embodiments, using variable region CD33 antibody sequences derived from 5′ RACE (rapid cloning of cDNA ends) cloning and the CD3 sequence from AMG 330, bispecific molecules can be assembled by synthesizing each scFv as a DNA fragment with overlapping Gibson assembly-compatible ends in the canonical bispecific T-cell engaging antibody format. Prototypical intervening regions such as 3xG₄S (SEQ ID NO: 158) linkers can be used between paired variable domains and a short 1xG₄S (SEQ ID NO: 159) linker between the two scFvs.

Vector can be mixed with psPAX2 packaging and pMD2. G envelope vectors and complexed with PEI to transfect suspension adapted HEK293T cells in 96-deep well blocks. Lentivirus can be harvested and then used to transduce Freestyle® 293-F cells in 96-deep well format and the cells are allowed to grow until viability begins to decline. IRES-driven GFP reporter expression can be monitored by flow cytometry to track target expression and identify failed transductions or weak expressers. Following incubation, conditioned media can be harvested and proteins can be purified using Protino® 96-sample Ni-NTA purification plates. Typical yields for bispecific T-cell engaging antibody construct proteins at this scale range from 50-200 μg.

In particular embodiments, the antibodies can also be formed as immunoconjugates. Immunoconjugates include an anti-CD33 antibody disclosed herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes (i.e., a radioimmunoconjugate). A toxin can be any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof. Other toxins include, for example, ricin, CC-1065 and analogues, the duocarmycins. Still other toxins include diptheria toxin, and snake venom (e.g., cobra venom). The toxin may be obtained from essentially any source; it may be synthetic or a natural product isolated from a selected source, e.g., a plant, bacterial, insect, mammalian or fungal source. The toxin may also be a synthetically modified natural product or an analogue of a natural product. Frequently used plant toxins are divided into two classes: (1) holotoxins (or class Il ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325(2001). The toxin may also be an antibody or other peptide.

Examples of radioactive isotopes that can be conjugated to antibodies of the present disclosure include iodine-131, indium-111, yttrium-90, and lutetium-177, as well as alpha-emitting radionuclides such as astatine-211 or bismuth-212 or bismuth-213. Methods for preparing radioimmunoconjugates are established in the art. Examples of radioimmunoconjugates are commercially available, including Zevalin™ (DEC Pharmaceuticals), and similar methods can be used to prepare radioimmunoconjugates using the antibodies of the disclosure.

Immunoconjugates allow for the targeted delivery of a drug moiety to cancer cell, and, in particular embodiments intracellular accumulation therein, where systemic administration of unconjugated drugs may result in unacceptable levels of toxicity to normal cells (Polakis P. (2005) Current Opinion in Pharmacology 5:382-387).

Immunoconjugates include antibody-drug conjugates (ADC). ADC are targeted chemotherapeutic molecules which combine properties of both antibodies and cytotoxic drugs by targeting potent cytotoxic drugs to antigen-expressing cancer cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3): 154-169; Chari, R. V. (2008) Acc. Chem. Res. 41:98-107). See also Kamath & lyer (Pharm Res. 32(11): 3470-3479, 2015), which describes considerations for the development of ADCs.

ADC compounds of the disclosure include those with anticancer activity. In particular embodiments, the ADC compounds include an antibody conjugated, i.e. covalently attached, to the drug moiety. In particular embodiments, the antibody is covalently attached to the drug moiety through a linker. A linker can include any chemical moiety that is capable of linking an antibody, antibody fragment (e.g., antigen binding fragments) or functional equivalent to another moiety, such as a drug moiety. Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the antibody remains active. Alternatively, linkers can be substantially resistant to cleavage (e.g., stable linker or noncleavable linker). In some aspects, the linker is a procharged linker, a hydrophilic linker, or a dicarboxylic acidObased linker. The ADCs selectively deliver an effective dose of a drug to cancer cells whereby greater selectivity, i.e. a lower efficacious dose, may be achieved while increasing the therapeutic index (“therapeutic window”).

The drug moiety (D) of the ADC may include any compound, moiety or group that has a cytotoxic or cytostatic effect. Drug moieties may impart their cytotoxic and cytostatic effects by mechanisms including tubulin binding, DNA binding or intercalation, and inhibition of RNA polymerase, protein synthesis, and/or topoisomerase. Exemplary drug moieties include maytansinoid (including monomethyl auristatin E [MMAE]; vedotin), dolastatin, auristatin, calicheamicin, pyrrolobenzodiazepine (PBD), nemorubicin and its derivatives, PNU-159682, anthracycline, duocarmycin, vinca alkaloid, taxane, trichothecene, CC1065, camptothecin, elinafide, and stereoisomers, isosteres, analogs, and derivatives thereof that have cytotoxic activity.

To prepare ADCs, linker-cytotoxin conjugates can be made by conventional methods analogous to those described by Doronina et al. (Bioconjugate Chem. 17:114-124, 2006). Antibodies, e.g., monoclonal antibodies, can be raised against a specific cancer target antigen (e.g., CD33), purified, and characterized. Therapeutic ADCs containing that antibody can be prepared by standard methods for cysteine conjugation, such as by methods analogous to that described in Hamblett et al., Clin. Cancer Res. 10:7063-7070, 2004; Doronina et al., Nat. Biotechnol. 21(7): 778-784, 2003; and Francisco et al., Blood 102:1458-1465. 2003.

Antibody-drug conjugates with multiple (e.g., four) drugs per antibody can be prepared by partial reduction of the antibody with an excess of a reducing reagent such as dithiothreitol (DTT) or tris (2-carboxyethyl) phosphine (TCEP) at 37°° C. for 30 min, then the buffer can be exchanged by elution through the gel filtration polymer SEPHADEX® (Cytiva Bioprocess (Uppsala, Sweden)) G-25 with 1 mM DTPA in Dulbecco's phosphate-buffered saline (DPBS). The eluent can be diluted with further DPBS, and the thiol concentration of the antibody can be measured using 5,5′-dithiobis (2-nitrobenzoic acid) [Ellman's reagent]. An excess, for example 5-fold, of the linker-cytotoxin conjugate can be added at 4° C. for 1 hr, and the conjugation reaction can be quenched by addition of a substantial excess, for example 20-fold, of cysteine. The resulting ADC mixture can be purified on SEPHADEX® G-25 equilibrated in PBS to remove unreacted linker-cytotoxin conjugate, desalted if desired, and purified by size-exclusion chromatography. The resulting ADC can then be sterile filtered, for example, through a 0.2 μm filter, and can be lyophilized if desired for storage.

Any of the antibodies described herein in any exemplary format can be formulated alone or in combination into compositions for administration to subjects. Salts and/or pro-drugs of the antibodies can also be used.

A pharmaceutically acceptable salt includes any salt that retains the activity of the antibody and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

In particular embodiments, the compositions include antibodies of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the antibodies or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, a-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous administration and more particularly by intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid formulations such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of the composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Depot formulations can include a variety of bioerodible polymers including poly(lactide), poly(glycolide), poly (caprolactone) and poly (lactide)-co(glycolide) (PLG) of desirable lactide: glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl) methyl cellulose (HPMC), HPMC acetate, cellulose acetate, pluronics (e.g., F68/F127), polysorbates, Span® (Croda Americas, Wilmington, Delaware), poly (vinyl alcohol) (PVA), Brij® (Croda Americas, Wilmington, Delaware), sucrose acetate isobutyrate (SAIB), salts, and buffers.

Excipients that partition into the external phase boundary of microparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot formulations can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

Methods of Use. Methods disclosed herein include treating subjects (humans, veterinary animals (dogs, cats, reptiles, birds, etc.) livestock (horses, cattle, goats, pigs, sheep, chickens, etc.) and research animals (monkeys, rats, mice, fish, etc.) with compositions disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of a CD33-related disorder's development or progression.

A “prophylactic treatment” includes a treatment administered to a subject who does not display signs or symptoms of a CD33-related (for instance, CD33-expressing) disorder or displays only early signs or symptoms of a CD33-related disorder such that treatment is administered for the purpose of diminishing or decreasing the risk of developing the CD33-related disorder further. Thus, a prophylactic treatment functions as a preventative treatment against a CD33-related disorder.

A “therapeutic treatment” includes a treatment administered to a subject who displays symptoms or signs of a CD33-related disorder and is administered to the subject for the purpose of diminishing or eliminating those signs or symptoms of the CD33-related disorder. The therapeutic treatment can reduce, control, or eliminate the presence or activity of the CD33-related disorder and/or reduce control or eliminate side effects of the CD33-related disorder.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, an increase in life expectancy, induced chemo-or radiosensitivity in cancer cells, inhibited cancer cell proliferation, prolonged subject life, reduced cancer-associated pain, and/or reduced relapse or re-occurrence of cancer following treatment.

In particular embodiments, therapeutically effects amounts induce an immune response. The immune response can be against a cancer cell.

Examples of CD33-related disorders include hematological cancers such as leukemias and lymphomas and other myelo-or lymphoproliferative disorders.

Exemplary leukemias include acute lymphoblastic leukemia (ALL), acute myeloid leukemia (AML), chronic lymphoid leukemia, chronic myelogenous leukemia (CML), chronic myelomonocytic leukemia (CML), lymphoid leukemia, mast cell leukemia, myelodysplastic syndrome (MDS), B-cell acute lymphoblastic leukemia (B-ALL), T-cell acute lymphoblastic leukemia (T-ALL), megakaryocytic leukemia, and undifferentiated leukemia.

Exemplary sub-types of AML include: acute basophilic leukemia, acute erythroid leukemia (AML-M6), acute megakaryoblastic leukemia (AML-M7), acute monoblastic leukemia (AML-M5a), acute monocytic leukemia (AML-M5b), acute myeloblasts leukemia with granulocytic maturation, acute myeloblasts leukemia without maturation, acute myelomonocytic leukemia (AML-M4), acute panmyelosis with myelofibrosis, acute promyelocytic leukemia (APL), erythroleukemia (AML-M6a), minimally differentiated acute myeloblasts leukemia, myelomonocytic leukemia with bone marrow eosinophilia, and pure erythroid leukemia (AML-M6b).

Exemplary lymphomas include Burkitt's lymphoma, diffuse large B-cell lymphoma, follicular lymphoma (small cell and large cell), Hodgkin's and non-Hodgkin's lymphoma, mantle cell lymphoma, and multiple myeloma.

Compositions disclosed herein can also be used to treat a complication or disease related to the above-noted lymphoproliferative disorders and hematological cancers. For example, complications relating to AML may include a preceding myelodysplastic syndrome (MDS, formerly known as “preleukemia”), secondary leukemia, in particular secondary AML, high white blood cell count, and absence of Auer rods. Among others, leukostasis and involvement of the central nervous system (CNS), hyperleukocytosis, residual disease, are also considered complications or diseases related to AML.

Compositions disclosed herein can may also find use in the treatment of other pathological conditions or genetic syndromes associated with the risk of AML such as Down syndrome, trisomy, Fanconi anemia, Bloom syndrome, Ataxia-telangiectasia, Diamond-Blackfan anemia, Schwachman-Diamond syndrome, Li-Fraumeni syndrome, Neurofibromatosis type 1, Severe congenital neutropenia (also called Kostmann syndrome).

Compositions disclosed herein may also find use in the treatment of Alzheimer's disease.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of CD33-related disorder, stage of CD33-related disorder, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 g/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2months, every 3 months, every 4 months, every 5 months, every 6 months, every 7 months, every 8 months, every 9 months, every 10 months, every 11 months or yearly).

The pharmaceutical compositions described herein can be administered by injection, inhalation, infusion, perfusion, lavage or ingestion. Routes of administration can include intravenous, intradermal, intraarterial, intraparenteral, intranasal, intranodal, intralymphatic, intraperitoneal, intralesional, intraprostatic, intravaginal, intrarectal, topical, intrathecal, intratumoral, intramuscular, intravesicular, oral, subcutaneous, and/or sublingual administration and more particularly by intravenous, intradermal, intraarterial, intraparenteral, intranasal, intranodal, intralymphatic, intraperitoneal, intralesional, intraprostatic, intravaginal, intrarectal, topical, intrathecal, intratumoral, intramuscular, intravesicular, oral, subcutaneous, and/or sublingual injection.

The Exemplary Embodiments and Example below are included to demonstrate particular, non-limiting embodiments of the disclosure. Those of ordinary skill in the art will recognize in light of the present disclosure that many changes can be made to the specific embodiments disclosed herein and still obtain a like or similar result without departing from the spirit and scope of the disclosure.

### Exemplary Embodiments

1. An isolated antibody or binding-competent fragment thereof comprising a binding domain specific for an epitope in a CD33 domain, and comprising at least one of:
(i) a V_{H} domain comprising the following CDRs:
   SEQ ID NO: 85, SEQ ID NO: 86, and SEQ ID NO: 87; or
   SEQ ID NO: 130, SEQ ID NO: 131, and SEQ ID NO: 132; or
   SEQ ID NO: 148, SEQ ID NO: 149, and SEQ ID NO: 150; or
   SEQ ID NO: 106, SEQ ID NO: 107, and SEQ ID NO: 108; or
   SEQ ID NO: 112, SEQ ID NO: 113, and SEQ ID NO: 114; or
   SEQ ID NO: 118, SEQ ID NO: 119, and SEQ ID NO: 120; or
   SEQ ID NO: 124, SEQ ID NO: 125, and SEQ ID NO: 126; or
   SEQ ID NO: 136, SEQ ID NO: 137, and SEQ ID NO: 138; or
   SEQ ID NO: 142, SEQ ID NO: 143, and SEQ ID NO: 144; or
   SEQ ID NO: 94, SEQ ID NO: 95, and SEQ ID NO: 96; or
   SEQ ID NO: 100, SEQ ID NO: 101, ad SEQ ID NO: 102;
and
(ii) a V_{L} domain comprising the following CDRs:
   SEQ ID NO: 88, SEQ ID NO: 89, and SEQ ID NO: 90; or
   SEQ ID NO: 133, SEQ ID NO: 134, and SEQ ID NO: 135; or
   SEQ ID NO: 151, SEQ ID NO: 152, and SEQ ID NO: 153; or
   SEQ ID NO: 109, SEQ ID NO: 110, and SEQ ID NO: 111; or
   SEQ ID NO: 115, SEQ ID NO: 116, and SEQ ID NO: 117; or
   SEQ ID NO: 121, SEQ ID NO: 122, and SEQ ID NO: 123; or
   SEQ ID NO: 127, SEQ ID NO: 128, and SEQ ID NO: 129; or
   SEQ ID NO: 139, SEQ ID NO: 140, and SEQ ID NO: 141; or
   SEQ ID NO: 145, SEQ ID NO: 146, and SEQ ID NO: 147; or
   SEQ ID NO: 97, SEQ ID NO: 98, and SEQ ID NO: 99; or
   SEQ ID NO: 103, SEQ ID NO: 104, and SEQ ID NO: 105.
2. The antibody or fragment thereof of embodiment 1, which binds the C2-set Ig-like domain of CD33, comprising:
   SEQ ID NO: 85, SEQ ID NO: 86, SEQ ID NO: 87, SEQ ID NO: 88, SEQ ID NO: 89, and SEQ ID NO: 90; or
   SEQ ID NO: 130, SEQ ID NO: 131, SEQ ID NO: 132, SEQ ID NO: 133, SEQ ID NO: 134, and SEQ ID NO: 135; or
   SEQ ID NO: 148, SEQ ID NO: 149, SEQ ID NO: 150, SEQ ID NO: 151, SEQ ID NO: 152, and SEQ ID NO: 153.
3. The antibody or fragment thereof of embodiment 2, comprising SEQ ID NO: 83 and SEQ ID NO: 84.
4. The antibody or fragment thereof of embodiment 2, comprising SEQ ID NO: 20 and SEQ ID NO: 21.
5. The antibody or fragment thereof of embodiment 2, comprising SEQ ID NO: 154 and SEQ ID NO: 155.
6. The antibody of embodiment 2, comprising 1H7, 5E10.7, or 2D5.
7. The antibody or fragment thereof of embodiment 1, which binds the C2-set Ig-like domain only in the absence of the V-set Ig-like domain, comprising:
   SEQ ID NO: 106, SEQ ID NO: 107, SEQ ID NO: 108, SEQ ID NO: 109, SEQ ID NO: 110, and SEQ ID NO: 111;
   SEQ ID NO: 112, SEQ ID NO: 113, SEQ ID NO: 114, SEQ ID NO: 115, SEQ ID NO: 116, and SEQ ID NO: 117;
   SEQ ID NO: 118, SEQ ID NO: 119, SEQ ID NO: 120, SEQ ID NO: 121, SEQ ID NO: 122, and SEQ ID NO: 123; SEQ ID NO: 124, SEQ ID NO: 125, SEQ ID NO: 126, SEQ ID NO: 127, SEQ ID NO: 128, and SEQ ID NO: 129; SEQ ID NO: 136, SEQ ID NO: 137, SEQ ID NO: 138, SEQ ID NO: 139, SEQ ID NO: 140, and SEQ ID NO: 141; or
   SEQ ID NO: 142, SEQ ID NO: 143, SEQ ID NO: 144, SEQ ID NO: 145, SEQ ID NO: 146, and SEQ ID NO: 147
8. The antibody or fragment thereof of embodiment 7, comprising SEQ ID NO: 12 and SEQ ID NO: 13.
9. The antibody or fragment thereof of embodiment 7, comprising SEQ ID NO: 14 and SEQ ID NO: 15.
10. The antibody or fragment thereof of embodiment 7, comprising SEQ ID NO: 16 and SEQ ID NO: 17.
11. The antibody or fragment thereof of embodiment 7, comprising SEQ ID NO: 18 and SEQ ID NO: 19.
12. The antibody or fragment thereof of embodiment 7, comprising SEQ ID NO: 22 and SEQ ID NO: 23.
13. The antibody or fragment thereof of embodiment 7, comprising SEQ ID NO: 24 and SEQ ID NO: 25.
14. The antibody of embodiment 7, comprising 12B12, 4H10, 11D5, 13E11, 11D11, or 7E7.
15. The antibody or fragment thereof of embodiment 1, which binds the V-set Ig-like domain of CD33, comprising:
   SEQ ID NO: 94, SEQ ID NO: 95, SEQ ID NO: 96, SEQ ID NO: 97, SEQ ID NO: 98, and SEQ ID NO: 99; or
   SEQ ID NO: 100, SEQ ID NO: 101, SEQ ID NO: 102, SEQ ID NO: 103, SEQ ID NO: 104, and SEQ ID NO: 105.
16. The antibody or fragment thereof of embodiment 15, comprising SEQ ID NO: 8 and SEQ ID NO: 9.
17. The antibody or fragment thereof of embodiment 15, comprising SEQ ID NO: 10 and SEQ ID NO: 11.
18. The antibody of embodiment 15, comprising 5D12 or 8F5.
19. The antibody or fragment thereof of any of embodiments 1-18 which is in the form of a whole antibody.
20. The antibody or fragment thereof of any one of embodiments 1-5, 7-13, or 15-17 which is in the form of a scFv.
21. A bi-specific antibody construct comprising a binding domain comprising an antibody fragment of any one of embodiment 1-5, 7-13, or 15-17.
22. The bi-specific antibody construct of embodiment 21, further comprising an immune cell activation epitope binding domain.
23. A chimeric antigen receptor (CAR) comprising a binding domain comprising an antibody fragment of any one of embodiments 1-5, 7-13, or 15-17, and an effector domain.
24. An scFV comprising a binding domain of (i) 5E10.7, 1H7, 12B12, 4H10, 11D5, 13E11, 11D11, 7E7, 5D12, 8F5, or 2D5; and (ii) an immune cell activator.
25. The scFV of embodiment 24, wherein the immune cell activator binding domain binds an epitope on CD3, CD28, or CD8.
26. The scFV of embodiment 24 wherein the immune cell activator binding domain comprises a CDR sequence of OKT3, OKT8, or 9D7.
27. The scFV of embodiment 24, comprising SEQ ID NO: 91.
28. An IgG-based bispecific antibody construct, comprising a binding domain of (i) 5E10.7, 1H7, 12B12, 4H10, 11D5, 13E11, 11D11, 7E7, 5D12, 8F5, or 2D5; and (ii) an immune cell activator.
29. The IgG-based bispecific antibody construct of embodiment 28, wherein the immune cell activator binding domain binds an epitope on CD3, CD28, or CD8.
30. The IgG-based bispecific antibody construct of embodiment 28, comprising SEQ ID NOs: 92and 93.
31. A chimeric antigen receptor (CAR) comprising a binding domain of (i) 5E10.7, 1H7, 12B12, 4H10, 11D5, 13E11, 11D11, 7E7, 5D12, 8F5, or 2D5; and (ii) an effector domain.
32. The CAR of embodiment 23 or embodiment 31, comprising an effector domain from one or more of: 4-1BB, CD3ε, CD3δ, CD3ζ, CD27, CD28, CD79A, CD79B, CARD11, DAP10, FcRα, FcRβ, FcRγ, Fyn, HVEM, ICOS, Lck, LAG3, LAT, LRP, NOTCH1, Wnt, NKG2D, OX40, ROR2, Ryk, SLAMF1, Slp76, pTα, TCRα, TCRγ, TRIM, Zap70, or PTCH2.
33. An antibody or binding-competent fragment thereof that binds a membrane-proximal epitope on CD33 that comprises at least one of residues 145-260 of SEQ ID NO: 36.
34. An anti-CD33 antibody or binding-competent fragment thereof essentially as described herein.
35. An immunoconjugate comprising an antibody, fragment, CAR, IgG-based bispecific antibody construct, or scFV of any of embodiments 1-35 conjugated to a cytotoxic agent.
36. A composition comprising an antibody, fragment, CAR, IgG-based bispecific antibody construct, scFV, or immunoconjugate of any of embodiments 1-36.
37. The composition of embodiment 36, further comprising a pharmaceutically acceptable excipient.
38. A method of treating a CD33-related disorder in a subject in need thereof, the method comprising administering a therapeutically amount of a composition of embodiment 36 or embodiment 37 to the subject, thereby treating the CD33-related disorder in the subject.
39. The method of embodiment 38, wherein the CD33-related disorder is a lymphoma or leukemia.
40. The method of embodiment 39, wherein the leukemia is acute myeloid leukemia (AML).

### Example 1. Identification and Characterization of CD33-specific Antibodies

FIGS. 4A-4C illustrate the expression (FIGS. 4A, 4B) and characterization of two CD33^{FL} specific (8F5 and 5D12) and one CD33^{ΔE2} specific (12B12) antibodies. Affinities for these antibodies (shown in FIG. 4C) were characterized by Biacore (Biacore, A&G Pharmaceutical, Inc., Columbia, MD) using purified antibodies and the corresponding soluble ectodomains. K_{D} for 8F5 is 8 nM; K_{D} for 12B12 is 8 nM, and Kp for 5D12 is 112 pM.

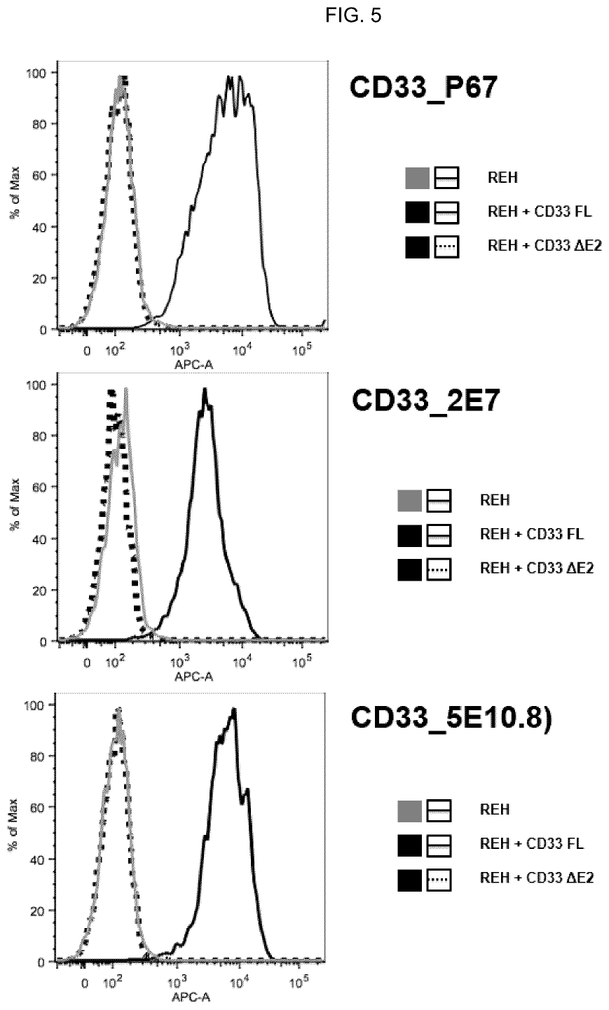

Most, if not all, therapeutic and diagnostic antibodies available prior to the developments are specific for the membrane distal V-set Ig-like domain of CD33 and therefore do not recognize the C2-set Ig-like domain and consequently also do not recognize the CD33^{ΔE2} isoform. FIG. 5 illustrates flow cytometry binding assay characterization of full-length CD33-specific antibodies (that is, which bind CD33^{FL}), compared with the commercially available anti-CD33 antibody P67.6. REH=human CD33^{neg} lymphoid cells.

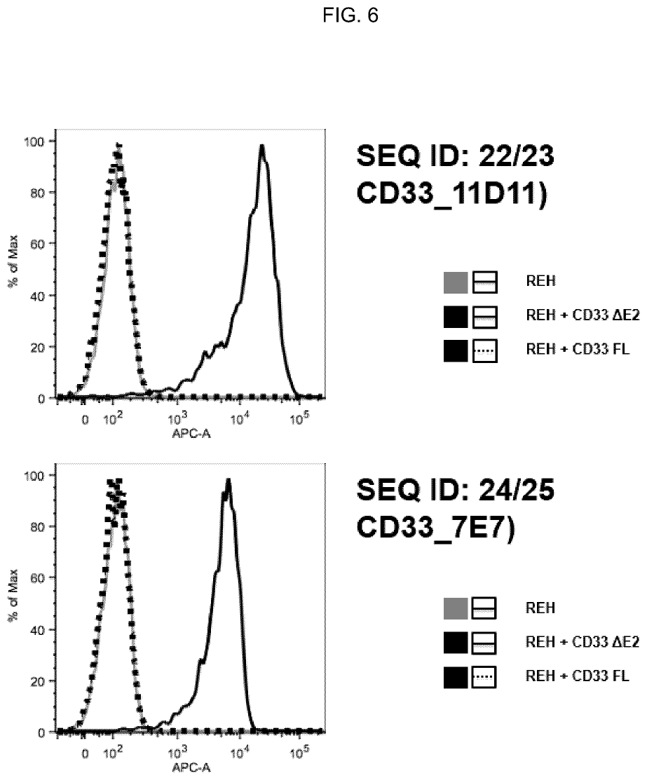

FIG. 6 illustrates flow cytometry binding assay characterization of CD33^{ΔE2} -specific CD33 antibodies. The binding assay shows novel antibodies (11D11 and 7E7) that bind the CD33^{ΔE2} isoform and not CD33^{FL}. Antibodies specific for CD33^{ΔE2} have not previously been described reliably in the literature. The herein-described CD33^{ΔE2} specific antibodies will be useful, including for determining expression profiles of this isoform across healthy and diseased individuals. REH=human CD33^{neg} lymphoid cells.

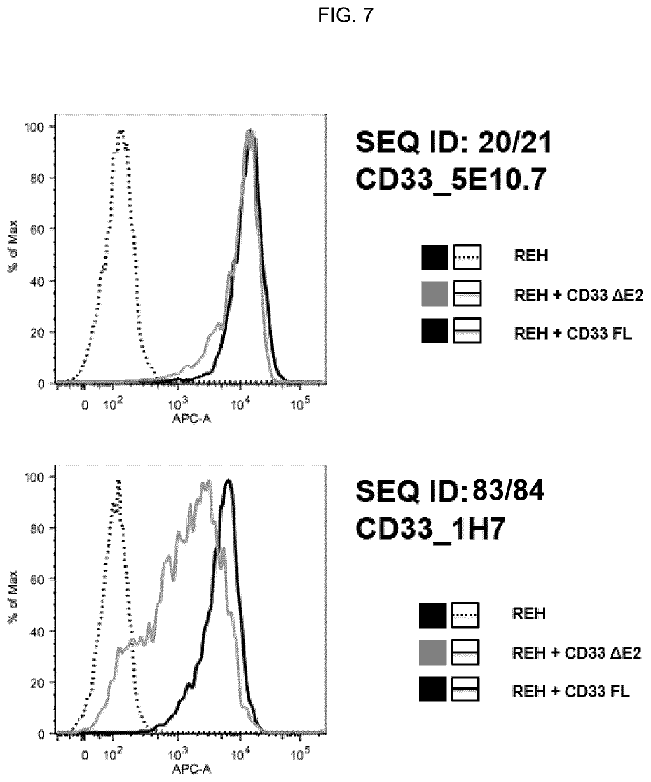

FIG. 7 illustrates flow cytometry binding assay characterization of pan specific CD33 antibodies. The binding assay shows novel antibodies (5E10.7 and 1H7) that bind the C2-set Ig-like domain and therefore recognize both CD33^{FL} and CD33^{ΔE2} . These antibodies and others with similar specificities are superior to those described in the literature because (1) they can target a higher density of CD33 antigen and (2) they bind a membrane proximal epitope which is predicted to yield, for example, more potent bispecific immune cell engaging antibodies and CAR-modified immune cells, which can also benefit from a membrane proximal epitope. REH=human CD33^{neg} lymphoid cells.

### Example 2. Expression and Purification of CD33^{FL} and CD33^{ΔE2} Isoforms

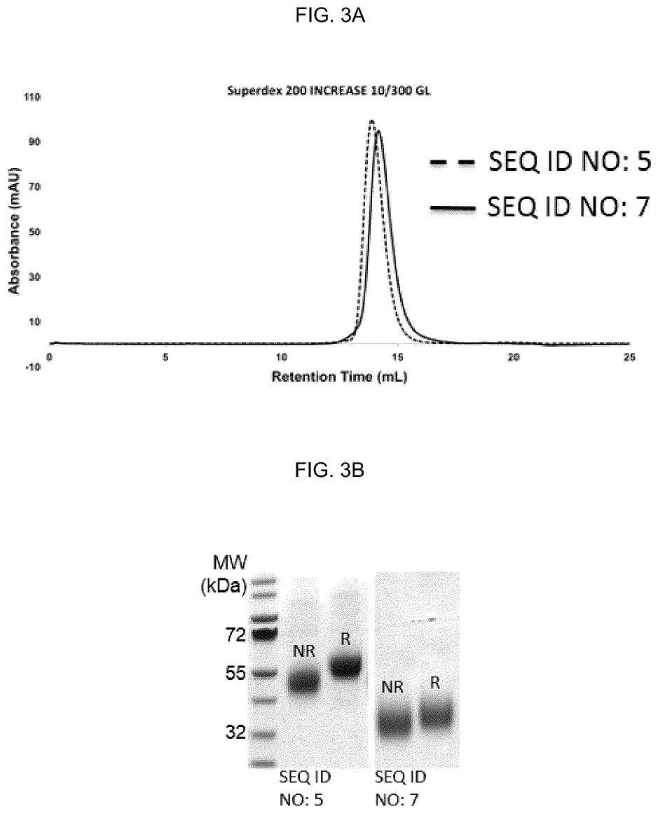

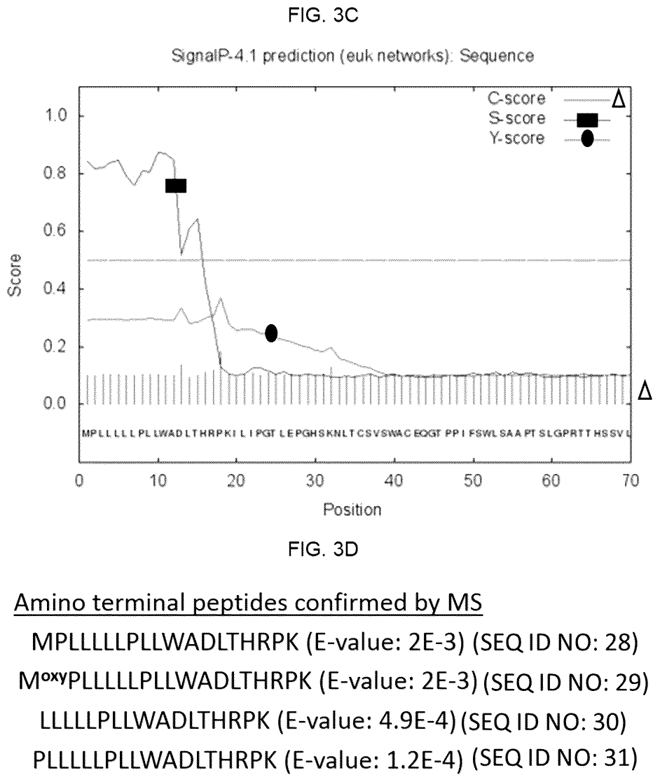

Extracellular versions of CD33^{FL} (SEQ ID NO: 5) and the CD33^{ΔE2} (SEQ ID NO: 7) isoforms were expressed, purified, and characterized. Size exclusion chromatography (FIG. 3A) and SDS-PAGE gel (FIG. 3B) show the purified recombinant proteins. FIG. 3C shows signal peptide prediction using the program SignalP (freely available on the World Wide Web at cbs.dtu.dk/services/SignalP/). This result highlights the fact that the leader peptide present on the CD33^{ΔE2} isoform is not recognizable as such (using SignalP) and therefore this portion of the isoform may represent a novel neoepitope. The sequence shown corresponds to positions 1-70 of SEQ ID NO: 2. FIG. 3D shows peptide sequences (SEQ ID NOs: 28-31) derived from the N-terminus of the recombinant CD33^{ΔE2} isoform as determined by mass spectrometry. Recombinant protein was reduced, alkylated and trypsinized to generate the data set.

### Example 3. Identification and Characterization of Additional CD33-Specific Antibodies

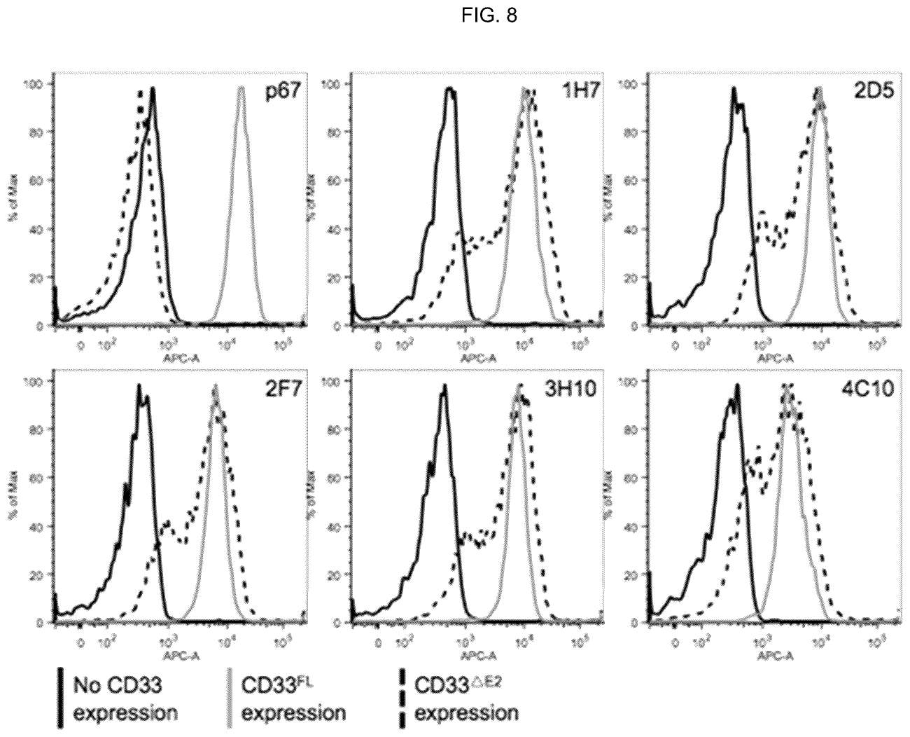

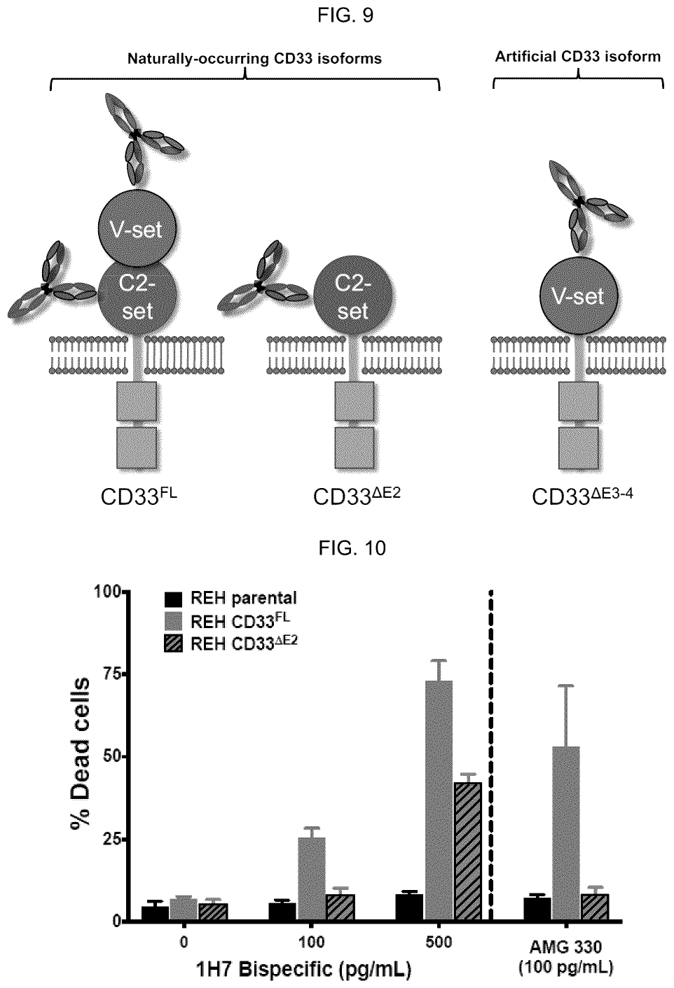

A series of C2-set domain-directed CD33 antibodies in mice were generated. As shown in FIG. 8, in REH (human CD33^{neg} lymphoid) cells and sublines engineered to express CD33^{ΔE2} or CD33^{FL}, five antibody clones (1H7, 2D5, 2F7, 3H10, and 4C10) recognized both CD33^{ΔE2} and CD33^{FL} and are therefore believed to bind the C2-set domain. Additional studies with 1H7 showed lack of binding to cells displaying an artificial CD33 molecule that contains only the V-set domain (CD33^{ΔE3-4} ; FIG. 9), confirming C2-set domain binding. CD334E3-E4 is an artificial, lab-generated CD33 molecule that contains only the V-set but not C2-set domain. This bring the V-set domain closer to the membrane and enables use of a V-set antibody (like AMG 330) to show that membrane proximal binding of therapeutic CD33 antibodies is superior to membrane-distal binding. This data supports the assertion that C2-set Ig-like domain antibodies (as describes herein, whether they bind only the C2-set domain alone or in the presence of V-set domain) have conceptual advantages over previously available antibodies.

Studies with a bispecific 1H7 antibody showed that CD33PAN antibodies indeed can exert cytotoxic properties against cells expressing CD33^{ΔE2} and CD33^{FL}, whereas the V-set domain-directed CD33/CD3 bispecific T-cell engager AMG 330 (Laszlo et al. Blood. 123(4): 554-561, 2014; Harrington et al. PLOS One. 10(8): e0135945, 2015) was only effective against CD33^{FL} -expressing cells (FIG. 10).

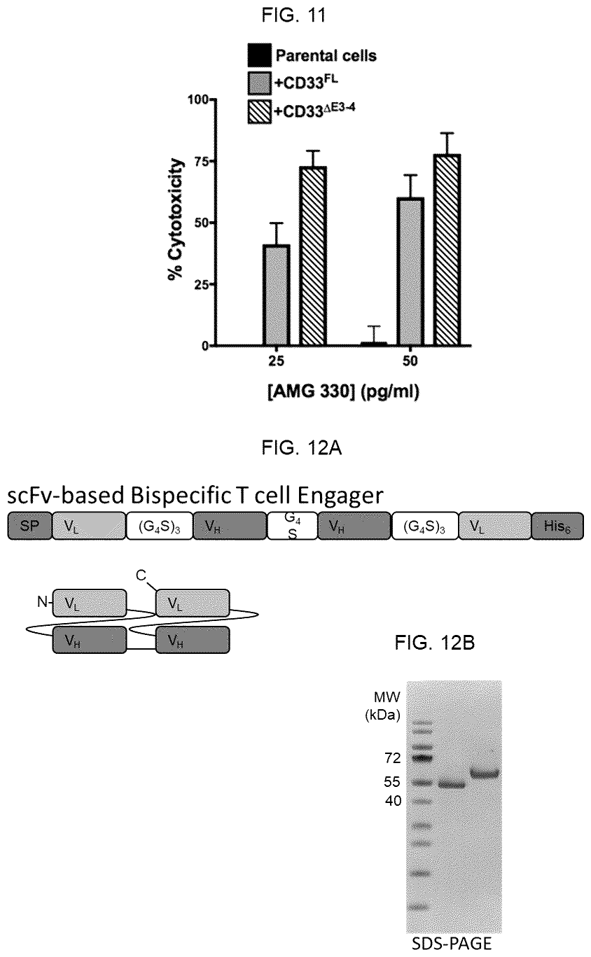

The importance of membrane proximity for therapeutic efficacy was initially suggested by data with CD20, CD22, CD25, and EpCAM antibodies (Cleary et al., J Immunol. 198(10): 3999-4011, 2017; Lin, Pharmgenomics Pers Med. 3:51-59, 2010; Haso et al., Blood. 121(7): 1165-1174, 2013; Bluemel et al., Cancer Immunol Immunother. 59(8): 1197-1209, 2010). Without being bound by any one theory, membrane-proximal targeting of CD33 can enhance the cytolytic activity of CAR T-cells. Consistent with this, preliminary studies in parental (CD33^{neg} ) human acute lymphoblastic RS4;11 cells and sublines engineered to express either CD33^{FL} or CD334E3-4 at the same cell surface density indicate greatly enhanced cytolytic activity AMG 330 (FIG. 11).

### Example 4. CAR with Selected CD33 Specificity

This example describes representative methods that can be used to produce CD33 CAR T-cells.

A lentiviral CAR construct can be assembled using scFv(s) from anti-CD33 antibodies described in the disclosure fused to, for instance, a CD28 transmembrane sequence and containing both 4-1BB and CD3 zeta chain signaling domains. The presence of these components will create a functional CAR receptor with CD33PAN specificity (where a CD33PAN antibody is used, though other anti-CD33 antibodies described herein can be used for different specificities). Lentiviral vectors can subsequently be prepared from these CD33 CAR constructs by transfection of the env, gag, pol and rev plasmids in a packaging cell line and tittered for functional assays.

### Example 5. In Vitro Assessment of CD33PAN CAR T-Cells-Directed Cytotoxicity Against AML and Myeloma Cells

This example describes representative methods that can be used for assessing, in vitro, the cytotoxicity of CD33PAN CAR T-cell.

Once a CD33PAN-directed CAR construct has successfully been created and expression confirmed, the ability of engineered T-cells expressing that construct to specifically kill CD33 expressing leukemia and myeloma cells will be tested, using a panel of acute leukemia and myeloma cell lines both as parental cells and sublines engineered to express either CD33^{ΔE2} or CD33^{FL}. Cell killing assays can be used to show efficacy of disclosed CARs using an assay to demonstrate CAR-T effectiveness, where target cells are adhered as a monolayer within individual wells of a 96-well plate. Each well contains microscopic electrodes covering the well floor with intact cells disrupting a small electrical current. This electrical current is then restored as cells are killed either through incubation with effector CAR modified T-cells at various concentrations or positive controls such as addition of Triton-X. This assay replaces previous versions which utilize flow cytometry-based analysis of cell viability markers or chromium release assays as a single endpoint readout. Alternatively, the assay allows for continuous real-time kinetics of cell killing over the course of days to directly compare head-to-head different constructs or tumor lines to one another and the relative rate of cell killing. Optimal concentration of tumor seeding can be empirically determined for each target line, and then growth kinetics can be tracked. Once standard curves have been established, modified T-cells from human donors expressing the CD33 CAR construct of interest can be added to target wells at concentrations ranging from 1:0.5 up to 1:20 (target: effector cell ratios) to observe for specific target cell killing as compared to either mock transduced or off-target modified T-cells. As confirmation of effective cell killing, secondary readout methods can be used to confirm cell cytotoxicity. including trypan blue exclusion and viability stain by flow cytometry, as well as microscopic imaging of effector wells.

### Example 6. In Vivo Assessment of Anti-Leukemia Activity of CD33^{PAN} CAR T-Cells using the Immunodeficient Mouse Model

This example describes representative methods that can be used for assessing, in vivo, the anti-leukemia activity of CD33^{PAN} CAR T-cells.

Using guidelines previously established for the generation of an immunotherapy humanized mouse model (Haworth et al., Mol Ther Methods Clin Dev. 6:17-30, 2017), neonate mice can first be infused with 1×10⁶ CD34+ HSPCs. Two different donor sources of HSPCs can be used for each experimental condition to account for donor-to-donor engraftment variability. Blood collection can occur every other week and analyzed by flow cytometry for peripheral human cell engraftment and lineage development. After T-cell development is observed in the peripheral blood of mice typically around 14-16 weeks post infusion, a portion of the mice can be sacrificed for total T-cell collection from blood and lymphoid compartments. Collected CD3+ can be stimulated with CD3/CD28 beads for 3-5 days and transduced with the corresponding CD33^{PAN} CAR lentiviral constructs. The first cohort of mice can include 4 mice per donor and can receive 1×10⁶ CAR-transduced CD3+ cells to verify that these cells can persist in the animals for several weeks without inducing GvHD.

Once successful, in cohorts 2 and 3 mice can then be divided into 3 different groups: i) mock mice receiving no cell injections, ii) mice receiving tumor cells alone, iii) mice receiving both tumor and modified T-cells. For this purpose, tumor cell lines can be generated that are transduced with a firefly luciferase to allow in vivo tumor burden monitoring. These tumor cells can be administered through intravenous injection 2-5 days prior to modified T-cell infusion in order to better mimic the clinical situation where tumor cells are already present in the patient. After infusion, mice will be monitored for both tumor burden using direct in vivo luciferase imaging. Additionally, peripheral blood will be analyzed by quantitative PCR to measure modified T-cell levels after infusion. T-cell subset composition will also be determined by flow cytometry phenotypic analysis.

The anti-leukemia activity of CD33^{PAN} CAR CD3+ cells will be verified by overall survival and by dissemination/growth of the tumor cell burden. At time of necropsy, animals can be sacrificed and multiple lymphoid tissues can be collected for analysis including bone marrow, spleen, and thymus. The frequency of modified cells can be quantified in each tissue by flow cytometry and validated by PCR based TaqMan methods. Tissues can also be assessed using both flow cytometry and quantitative PCR for the presence of residual tumor cells using methods for specifically detecting the luciferase gene present in the tumor lines.

### Example 7. Production of 1H7-Based Bi-Specific T-Cell Engaging Antibodies

This example describes the production of a CD33/CD3-directed scFv-based bi-specific T-cell engaging antibody and a CD33/CD3-directed IgG-based bi-specific T-cell engaging antibody.

Representative bi-specific T-cell engaging antibodies based on the 1H7 antibody were produced and expressed. Protein sequences were reverse-translated and codon optimized for gene synthesis. The plasmids encoding the genetic expression constructs were packaged into lentiviral particles, which were then used to transduce HEK293F producer cells. The proteins were secreted into the culture medium and purified using NiNTA affinity chromatography. The proteins were then further purified by size exclusion chromatography and quantitated using standard techniques.

FIG. 12A illustrates the structure of a representative scFv-based bispecific T-cell engaging antibody (linear and folded). FIG. 12B is a SDS-PAGE gel showing expression of the 1H7-CD3 bispecific T-cell engaging antibody shown in SEQ ID NO: 91; the 1H7 antibody sequences used to build this construct are SEQ ID NOs: 83 and 84. In SEQ ID NO: 91, positions 21-259 correspond to the scFv for 1H7, in VL-VH orientation separated by a linker (positions 128-142); and positions 165-513 correspond to the scFv for anti-CD33 (derived from AMG330; including a linker at positions 390-404).

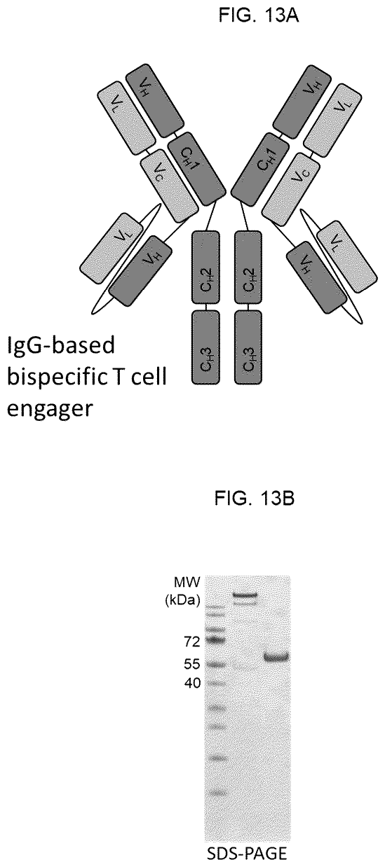

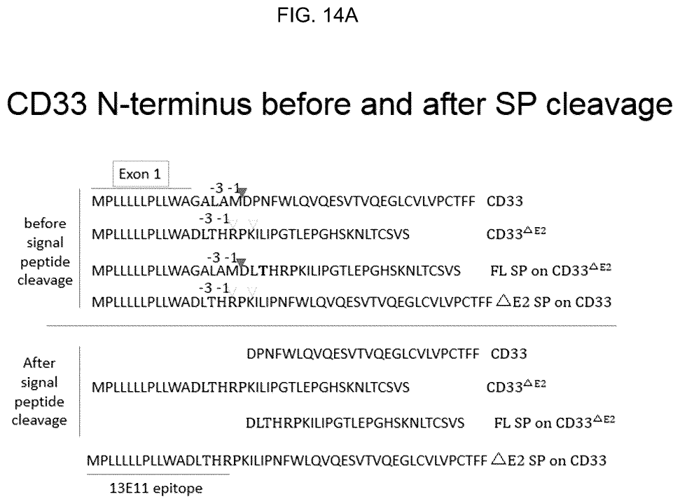

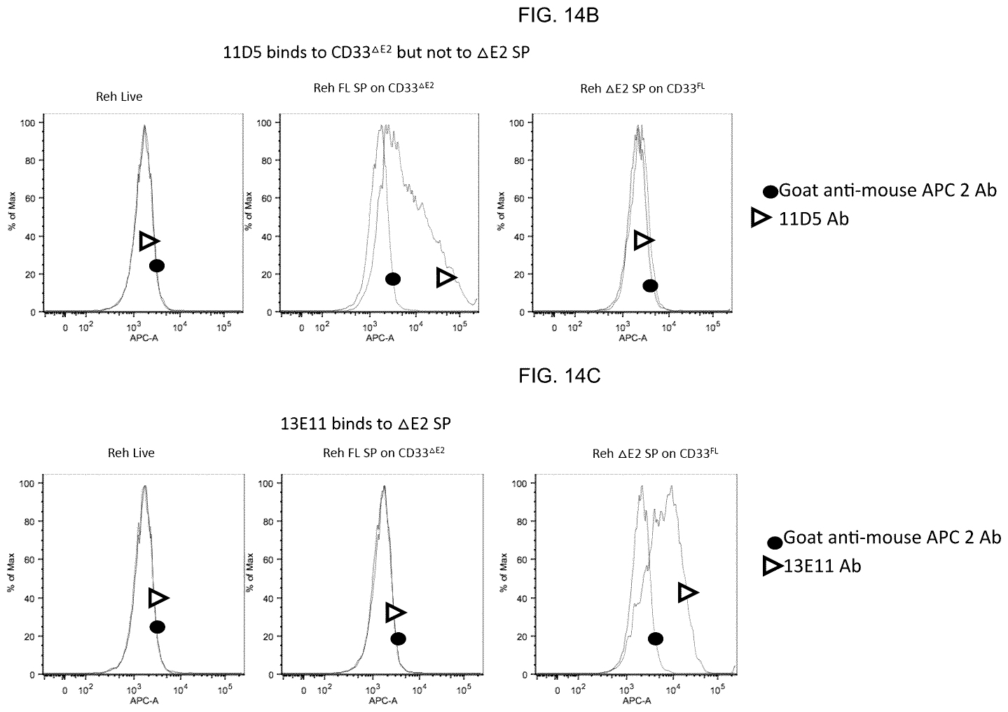

FIG. 13A illustrates the folded and assembled structure of a representative IgG-based bispecific T-cell engaging antibody construct. FIG. 13B is a SDS-PAGE gel showing expression of the 1H7-CD3 bispecific T-cell engaging antibody shown in SEQ ID NOs: 92 and 93; the 1H7 antibody light and heavy chain sequences used to build these constructs are SEQ ID NOs: 84and 83 (respectively). In SEQ ID NO: 92, positions 21-234 correspond to the light chain for 1H7; positions 250-498 correspond to the scFv for anti-CD3 (derived from AMG330, as above); a linker separates these segments. In SEQ ID NO: 93, the heavy chain for 1H7 is positions 21-467.

### Example 8. Characterization of Antibody Binding to CD33 Signal Peptides in Hybrid Proteins

This example describes characterization of antigen binding to neoepitope(s) in CD33^{ΔE2} formed due to a retained signal peptide on the amino terminal of CD33^{ΔE2} .

Hybrid proteins were produced wherein the signal peptide of native CD33 was swapped with that of CD33^{ΔE2} and vice versa. The binding of two antibodies, 11D5 and 13E11, to these hybrid proteins was then analyzed.

FIG. 14A is a sequence illustration of the C-terminal end (including the signal peptide) of native CD33^{FL}, CD33^{ΔE2} , a hybrid protein including the signal peptide of CD33^{FL} fused to the C-terminal end of CD33^{ΔE2} (FL SP on CD33^{ΔE2}), and a hybrid protein including the signal peptide of CD33^{ΔE2} fused to the C-terminal end of CD33 FL (ΔE2 SP on CD33^{FL}). The upper section of FIG. 14A shows the sequences before signal peptide cleavage; the lower section, after such cleavage (as proposed). Signal peptide cleavage are sites indicated with filled triangle. Predicted possible signal peptide cleavage sites indicated with dotted empty triangles. The sequence comprising a 13E11 epitope is indicated. In order, the sequences shown in FIG. 14A correspond to:
residues 1-44 of SEQ ID NO: 1 (exon 1=positions 1-12);
residues 1-39 of SEQ ID NO: 2 (exon 1=positions 1-12);
SEQ ID NO: 156 (exon 1=positions 1-12);
SEQ ID NO: 157 (exon 1=positions 1-12);
residues 18-44 of SEQ ID NO: 1;
residues 1-39 of SEQ ID NO: 2 (exon 1=positions 1-12);
residues 140-166 of SEQ ID NO: 1 (which is also 13-39 of SEQ ID NO: 2); and
SEQ ID NO: 157 (exon 1=positions 1-12).

FIGS. 14B-14C show REH cells engineered to express hybrid CD33 proteins. 11D5, a CD33^{ΔE2} -specific antibody that cannot bind to CD33^{FL}, can bind to CD33^{ΔE2} in the absence of the ΔE2SP, and the signal peptide of CD33^{ΔE2} fused to the C-terminal end of CD33^{FL} (ΔE2 SP on CD33^{FL}) does not confer 11D5 binding. This indicates that 11D5 binds to CD33^{ΔE2} independent of the ΔE2 SP (FIG. 14B). 13E11, a CD334E-specific antibody that cannot bind to CD33^{FL}, also cannot bind to CD33^{ΔE2} in the absence of the ΔE2 SP. Furthermore, the signal peptide of CD33^{ΔE2} fused to the C-terminal end of CD33^{FL} (ΔE2 SP on CD33^{FL}) does confer 11D5 binding to CD33^{FL}, indicating that 13E11 binds to an epitope within the ΔE2 SP.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in binding between an antibody and antigen as compared to the values depicted in the accompanying FIGs.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of +20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

### Supporting Sequences

SEQ ID NO: 1 is the amino acid sequence of human full-length CD33 hsCD33-mmFc used as an immunogen for human FL (MDT000208):

MPLLLLLPLLWAGALAMDPNFWLQVQESVTVQEGLCVLVPCTFFHPIPYY
DKNSPVHGYWFREGAIISRDSPVATNKLDQEVQEETQGRFRLLGDPSRNN
CSLSIVDARRRDNGSYFFRMERGSTKYSYKSPQLSVHVTDLTHRPKILIP
GTLEPGHSKNLTCSVSWACEQGTPPIFSWLSAAPTSLGPRTTHSSVLIIT
PRPQDHGTNLTCQVKFAGAGVTTERTIQLNVTYVPQNPTTGIFPGDGSGK
QETRAGGGSGCKPCICTVPEVSSVFIFPPKPKDVLTITLTPKVTCVVVDI
SKDDPEVQFSWFVDDVEVHTAQTQPREEQFNSTFRSVSELPIMHQDWLNG
KEFKCRVNSAAFPAPIEKTISKTKGRPKAPQVYTIPPPKEQMAKDKVSLT
CMITDFFPEDITVEWQWNGQPAENYKNTQPIMNTNGSYFVYSKLNVQKSN
WEAGNTFTCSVLHEGLHNHHTEKSLSHSPGK

SEQ ID NO: 2 is the amino acid sequence of the human ΔE2 version of CD33 (CD33^{ΔE2}) with a mouse Fc domain (MDT000209; hsCD33_ΔE2-mmFc; Immunogen for human ΔE2):

MPLLLLLPLLWADLTHRPKILIPGTLEPGHSKNLTCSVSWACEQGTPPIF
SWLSAAPTSLGPRTTHSSVLIITPRPQDHGTNLTCQVKFAGAGVTTERTI
QLNVTYVPQNPTTGIFPGDGSGKQETRAGGGSGCKPCICTVPEVSSVFIF
PPKPKDVLTITLTPKVTCVVVDISKDDPEVQFSWFVDDVEVHTAQTQPRE
EQFNSTFRSVSELPIMHQDWLNGKEFKCRVNSAAFPAPIEKTIS

SEQ ID NO: 3 is the amino acid sequence of Macaca fascicularis CD33^{FL} with a mouse Fc domain (mfCD33-mmFc; MDT000210; Immunogen for cyno FL):

MPLLLLPLLWAGALAMDPRVRLEVQESVTVQEGLCVLVPCTFFHPVPYHT
RNSPVHGYWFREGAIVSLDSPVATNKLDQEVQEETQGRFRLLGDPSRNNC
SLSIVDARRRDNGSYFFRMEKGSTKYSYKSTQLSVHVTDLTHRPQILIPG
ALDPDHSKNLTCSVPWACEQGTPPIFSWMSAAPTSLGLRTTHSSVLIITP
RPQDHGTNLTCQVKFPGAGVTTERTIQLNVSYASQNPRTDIFLGDGSGRK
ARKQGGGSGCKPCICTVPEVSSVFIFPPKPKDVLTITLTPKVTCVVVDIS
KDDPEVQFSWFVDDVEVHTAQTQPREEQFNSTFRSVSELPIMHQDWLNGK
EFKCRVNSAAFPAPIEKTISKTKGRPKAPQVYTIPPPKEQMAKDKVSLTC
MITDFFPEDITVEWQWNGQPAENYKNTQPIMNTNGSYFVYSKLNVQKSNW
EAGNTFTCSVLHEGLHNHHTEKSLSHSPGK

SEQ ID NO: 4 is the amino acid sequence of the extracellular domain of mouse CD33^{FL} where the C2-set Ig-like domain from mouse has been replaced with the C2-set Ig-like domain from human CD33; combined with a Fc region from human IgG1 (mmCD33_Vset-mmCD33_C2set-hsCD33_Fc_hslgG1; MDT000524; Immunogen for human ΔE2):

MLWPLPLFLLCAGSLAQDLEFQLVAPESVTVEEGLCVHVPCSVFYPSIKL
TLGPVTGSWLRKGVSLHEDSPVATSDPRQLVQKATQGRFQLLGDPQKHDC
SLFIRDAQKNDTGMYFFRVVREPFVRYSYKKSQLSLHVTDLTHRPKILIP
GTLEPGHSKNLTCSVSWACEQGTPPIFSWLSAAPTSLGPRTTHSSVLIIT
PRPQDHGTNLTCQVKFAGAGVTTERTIQLNVTYVPQNPTTGIFPGDGSGK
QETRAGSEPKSSDKTHTCPPCPAPELLGGPSVFLFPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYS
KLTVDKSRWQQGNVFSCSVMHEALHNHYTQKSLSLSPGK

SEQ ID NO: 5 is the amino acid sequence of the extracellular domain of CD33^{FL} with a 6-histidine-avidin tag (hsCD33-His-Avi; MDT000235; Screening Reagent):

MPLLLLLPLLWAGALAMDPNFWLQVQESVTVQEGLCVLVPCTFFHPIPYY
DKNSPVHGYWFREGAIISRDSPVATNKLDQEVQEETQGRFRLLGDPSRNN
CSLSIVDARRRDNGSYFFRMERGSTKYSYKSPQLSVHVTDLTHRPKILIP
GTLEPGHSKNLTCSVSWACEQGTPPIFSWLSAAPTSLGPRTTHSSVLIIT
PRPQDHGTNLTCQVKFAGAGVTTERTIQLNVTYVPQNPTTGIFPGDGSGK
QETRAGSHHHHHHGSGLNDIFEAQKIEWHE

SEQ ID NO: 7 is the amino acid sequence of the extracellular domain of CD33^{ΔE2} with a 6-histidine-avidin tag (hsCD33_ΔE2-His-Avi; MDT000239; Screening Reagent):

MPLLLLPLLWAGALAMDPRVRLEVQESVTVQEGLCVLVPCTFFHPVPYHT
RNSPVHGYWFREGAIVSLDSPVATNKLDQEVQEETQGRFRLLGDPSRNNC
SLSIVDARRRDNGSYFFRMEKGSTKYSYKSTQLSVHVTDLTHRPQILIPG
ALDPDHSKNLTCSVPWACEQGTPPIFSWMSAAPTSLGLRTTHSSVLIITP
RPQDHGTNLTCQVKFPGAGVTTERTIQLNVSYASQNPRTDIFLGDGSGRK
ARKQGSHHHHHHGSGLNDIFEAQKIEWHE

SEQ ID NO: 7 is the amino acid sequence of hsCD33_ΔE2-His-Avi Screening Reagent (MDT000239):

MPLLLLLPLLWADLTHRPKILIPGTLEPGHSKNLTCSVSWASEQGTPPIF
SWLSAAPTSLGPRTTHSSVLIITPRPQDHGTNLTCQVKFAGAGVTTERTI
QLNVTYVPQNPTTGIFPGDGSGKQETRAGSHHHHHHGSGLNDIFEAQKIE
WHE

SEQ ID NO: 8 is the amino acid sequence of the light chain of IgG2a anti-CD33 antibody 5D12 (Ab_CD33_5D12_mmIgG2a_Light Chain; MDT000444), which is specific for CD33^{FL} :

METDTLLLWVLLLWVPGSTGDIKMTQSPSSIYASLGERVTINCKASQDIK
SYLSWYQQKPWKSPKTLIYYATTLADGVPSRFSGSGSGQDYSLTISSLES
DDTATYYCLHHGESPWTFGEGTKLEIKRADAAPTVSIFPPSSEQLTSGGA
SVVCFLNNFYPKDINVKWKIDGSERQNGVLNSWTDQDSKDSTYSMSSTLT
LTKDEYERHNSYTCEATHKTSTSPIVKSFNRNEC

SEQ ID NO: 9 is the amino acid sequence of the heavy chain of IgG2a anti-CD33 antibody 5D12 (Ab_CD33_5D12_mmIgG2a_Heavy Chain; MDT000445), which is specific for CD33^{FL} :

METDTLLLWVLLLWVPGSTGQVQLQQSGAEVVKPGASVKISCRASGYAFS
NYWMNWVKQRPGKGLEWIGQIYPGNFNTDYNGQFKGKATLTVDKSSNTAY
MQLSSLTSEDSAVYFCARFFDFGAYFTLDYWGQGTSVTVSSAKTTAPSVY
PLAPVCGDTTGSSVTLGCLVKGYFPEPVTLTWNSGSLSSGVHTFPAVLQS
DLYTLSSSVTVTSSTWPSQSITCNVAHPASSTKVDKKIEPRGPTIKPCPP
CKCPAPNLLGGPSVFIFPPKIKDVLMISLSPIVTCVVVDVSEDDPDVQIS
WFVNNVEVHTAQTQTHREDYNSTLRVVSALPIQHQDWMSGKEFKCKVNNK
DLPAPIERTISKPKGSVRAPQVYVLPPPEEEMTKKQVTLTCMVTDFMPED
IYVEWTNNGKTELNYKNTEPVLDSDGSYFMYSKLRVEKKNWVERNSYSCS
VVHEGLHNHHTTKSFSRTPGK

SEQ ID NO: 10 is the amino acid sequence of the light chain of IgG2a anti-CD33 antibody 8F5 (Ab_CD33_8F5_mmIgG2a_Light Chain; MDT000446), which is specific for CD33^{FL} :

METDTLLLWVLLLWVPGSTGDIVMSQSPSSLPVSVGEKVTLSCKSSQSLL
YSRNQYNFLAWYQQRPGQSPKLLIYWASTRESGVPDRFTGSGSGTDFTLT
ISSVKAEDLAVYYCQQYYSYPYTFGGGTKLEIKRADAAPTVSIFPPSSEQ
LTSGGASVVCFLNNFYPKDINVKWKIDGSERQNGVLNSWTDQDSKDSTYS
MSSTLTLTKDEYERHNSYTCEATHKTSTSPIVKSFNRNEC

SEQ ID NO: 11 is the amino acid sequence of the heavy chain of IgG2a anti-CD33 antibody 8F5 (Ab_CD33_8F5_mmIgG2a_Heavy Chain; MDT000446), which is specific for CD33^{FL} :

METDTLLLWVLLLWVPGSTGEVKLVESGGGLVQPGGSLKLSCAASG
FTFSDFYMYWVRQTPEKRLEWVAFISNAGVTTYYPDTVEGRFTISR
DNAKNTLYLQMSRLMSEDTAMYYCTKSDYDGAWFPYWGQGTLVTVS
AAKTTAPSVYPLAPVCGDTTGSSVTLGCLVKGYFPEPVTLTWNSGS
LSSGVHTFPAVLQSDLYTLSSSVTVTSSTWPSQSITCNVAHPASST
KVDKKIEPRGPTIKPCPPCKCPAPNLLGGPSVFIFPPKIKDVLMIS
LSPIVTCVVVDVSEDDPDVQISWFVNNVEVHTAQTQTHREDYNSTL
RVVSALPIQHQDWMSGKEFKCKVNNKDLPAPIERTISKPKGSVRAP
QVYVLPPPEEEMTKKQVTLTCMVTDFMPEDIYVEWTNNGKTELNYK
NTEPVLDSDGSYFMYSKLRVEKKNWVERNSYSCSVVHEGLHNHHTT
KSFSRTPGK

SEQ ID NO: 12 is the amino acid sequence of the light chain of IgG2b anti-CD33 antibody 12B12 (Ab_CD33_12B12_mmIgG2b_Light Chain; MDT000448), which is specific for CD33^{ΔE2}:

METDTLLLWVLLLWVPGSTGDIVMTQAAFSNPVTLGTSASISCRSS
QSLLHSNGITYLYWYLQKPGQSPQLLIYQMSNLASGVPDRESSSGS
GTDFTLRISRVEAEDVGVYYCAQNLELPPTFGGGTKLEIKRADAAP
TVSIFPPSSEQLTSGGASVVCFLNNFYPKDINVKWKIDGSERQNGV
LNSWTDQDSKDSTYSMSSTLTLTKDEYERHNSYTCEATHKTSTSPI
VKSFNRNEC

SEQ ID NO: 13 is the amino acid sequence of the heavy chain of IgG2b anti-CD33 antibody 12B12 (Ab_CD33_12B12_mmIgG2b_Heavy Chain; MDT000449), which is specific for CD33^{ΔE2}:

METDTLLLWVLLLWVPGSTGEVQLQQSGTVLARPGASVKMSCKASG
YTFTTYWMHWIKQSPGQGLEWIGAIYPGNSDTSYNQKFKGKAKLTA
VTSASTAYMELSSLTNEDSAVYYCEIYDGYHFIYWGQGTTLTVSSA
KTTPPSVYPLAPGCGDTTGSSVTLGCLVKGYFPESVTVTWNSGSLS
SSVHTFPALLQSGLYTMSSSVTVPSSTWPSQTVTCSVAHPASSTTV
DKKLEPSGPISTINPCPPCKECHKCPAPNLEGGPSVFIFPPNIKDV
LMISLTPKVTCVVVDVSEDDPDVQISWFVNNVEVHTAQTQTHREDY
NSTIRVVSTLPIQHQDWMSGKEFKCKVNNKDLPSPIERTISKIKGL
VRAPQVYILPPPAEQLSRKDVSLTCLVVGFNPGDISVEWTSNGHTE
ENYKDTAPVLDSDGSYFIYSKLNMKTSKWEKTDSFSCNVRHEGLKN
YYLKKTISRSPGK

SEQ ID NO: 14 is the amino acid sequence of the light chain of anti-CD33 antibody 4H10, isolated as IgG2a and reformatted as IgG1 for recombinant expression (Ab_CD33_4H10_mmIgG1_Light Chain; MDT000492), which is specific for CD33^{ΔE2}:

METDTLLLWVLLLWVPGSTGDVVMTQTPLTLSVTIGQPASISCKSS
QSLLYSNGKTYLHWFLQRPGQSPKRLIYLVSKLDSGVPDRFTGSGS
GTDFTLKISRVEAEDLGVYYCVQGTHFPRTFGGGTKLEIKADAAPT
VSIFPPSSEQLTSGGASVVCFLNNFYPKDINVKWKIDGSERQNGVL
NSWTDQDSKDSTYSMSSTLTLTKDEYERHNSYTCEATHKTSTSPIV
KSFNRNEC

SEQ ID NO: 15 is the amino acid sequence of the heavy chain of anti-CD33 antibody 4H10, isolated as IgG2a and reformatted as IgG1 for recombinant expression (Ab_CD33_4H10_mmIgG1_Heavy Chain; MDT000491), which is specific for CD33^{ΔE2}:

METDTLLLWVLLLWVPGSTGQVQLQQSGAELVRPGTSVKVSCKASG
YAFTNYLIEWVKQRPGQGLEWIGVIHPGNNSTSYNAKFRGKATLTA
DRSSSTAYMQLSSLTSEDSAVYFCARYGYDERNAMDYWGQGTSVTV
SSAKTTPPSVYPLAPGSAAQTNSMVTLGCLVKGYFPEPVTVTWNSG
SLSSGVHTFPAVLQSDLYTLSSSVTVPSSPRPSETVTCNVAHPASS
TKVDKKIVPRDCGCKPCICTVPEVSSVFIFPPKPKDVLTITLTPKV
TCVVVDISKDDPEVQFSWFVDDVEVHTAQTQPREEQFNSTFRSVSE
LPIMHQDWLNGKEFKCRVNSAAFPAPIEKTISKTKGRPKAPQVYTI
PPPKEQMAKDKVSLTCMITDFFPEDITVEWQWNGQPAENYKNTQPI
MNTNGSYFVYSKLNVQKSNWEAGNTFTCSVLHEGLHNHHTEKSLSH
SPGK

SEQ ID NO: 16 is the amino acid sequence of the light chain of IgG1 anti-CD33 antibody 11D5 (Ab_CD33_11D5_mmIgG1_Light Chain; MDT000494), which is specific for CD33^{ΔE2}:

METDTLLLWVLLLWVPGSTGDIVMTQAAFSNPVTLGTSASISCRSN
KSLLHSNGITYLYWYLQKPGQSPQLLIYQMSNLASGVPDRESSSGS
GTDFTLRISRVEAEDVGVYYCAQNLELPPTFGGGTKLEIKRADAAP
TVSIFPPSSEQLTSGGASVVCFLNNFYPKDINVKWKIDGSERQNGV
LNSWTDQDSKDSTYSMSSTLTLTKDEYERHNSYTCEATHKTSTSPI
VKSFNRNEC

SEQ ID NO: 17 is the amino acid sequence of the heavy chain of IgG1 anti-CD33 antibody 11D5 (Ab_CD33_11D5_mmIgG1_Heavy Chain; MDT000493), which is specific for CD33^{ΔE2}:

METDTLLLWVLLLWVPGSTGEVQFQQSETVLARPGTSVKLSCKASG
YTFTSYWMHWLKQRPGQGLEWIGAIYCGNSDTSYNQKFKGKAKLTA
VTSATTAYMELSSLTNEDSAVYYCKIYDGYHFDYWGQGTTLTVSSA
KTTPPSVYPLAPGSAAQTNSMVTLGCLVKGYFPEPVTVTWNSGSLS
SGVHTFPAVLQSDLYTLSSSVTVPSSPRPSETVTCNVAHPASSTKV
DKKIVPRDCGCKPCICTVPEVSSVFIFPPKPKDVLTITLTPKVTCV
VVDISKDDPEVQFSWFVDDVEVHTAQTQPREEQFNSTFRSVSELPI
MHQDWLNGKEFKCRVNSAAFPAPIEKTISKTKGRPKAPQVYTIPPP
KEQMAKDKVSLTCMITDFFPEDITVEWQWNGQPAENYKNTQPIMNT
NGSYFVYSKLNVQKSNWEAGNTFTCSVLHEGLHNHHTEKSLSHSPG
K

SEQ ID NO: 18 is the amino acid sequence of the light chain of IgG1 anti-CD33 antibody 13E11 (Ab_CD33_13E11_mmIgG1_Light Chain; MDT000499), which is specific for CD33^{ΔE2}:

METDTLLLWVLLLWVPGSTGDIVLTQSPVSLAVSLGQRATISCKAS
HGVEYAGAHYMNWYQQKPGQPPKLLIYAASNLGSGTDFTLNIHPVE
EEDSATYYCQQSNEDPRTFGGGTKLEIKRADAAPTVSIFPPSSEQL
TSGGASVVCFLNNFYPKDINVKWKIDGSERQNGVLNSWTDQDSKDS
TYSMSSTLTLTKDEYERHNSYTCEATHKTSTSPIVKSFNRNEC

SEQ ID NO: 19 is the amino acid sequence of the heavy chain of IgG1 anti-CD33 antibody 13E11 (Ab_CD33_13E11_mmIgG1_Heavy Chain; MDT000498), which is specific for CD33^{ΔE2}:

METDTLLLWVLLLWVPGSTGKVQLQQSGAELVKPGASVKLSCKASG
YTFTDYTLHWLKQRSGQGLEWIGWFYPTSGSINYNERFKDKATLTA
DKSSSTVYMELSRLTSVDSAVYFCARHKFGFDYWGQGTTLTVSSAK
TTPPSVYPLAPGSAAQTNSMVTLGCLVKGYFPEPVTVTWNSGSLSS
GVHTFPAVLQSDLYTLSSSVTVPSSPRPSETVTCNVAHPASSTKVD
KKIVPRDCGCKPCICTVPEVSSVFIFPPKPKDVLTITLTPKVTCVV
VDISKDDPEVQFSWFVDDVEVHTAQTQPREEQFNSTFRSVSELPIM
HQDWLNGKEFKCRVNSAAFPAPIEKTISKTKGRPKAPQVYTIPPPK
EQMAKDKVSLTCMITDFFPEDITVEWQWNGQPAENYKNTQPIMNTN
GSYFVYSKLNVQKSNWEAGNTFTCSVLHEGLHNHHTEKSLSHSPGK

SEQ ID NO: 20 is the amino acid sequence of the light chain of IgG1 anti-CD33 antibody 5E10.7 (Ab_CD33_5E10.7_mmIgG1_Light Chain; MDT000551), which is specific for CD33^{FL} and CD33^{ΔE2}:

METDTLLLWVLLLWVPGSTGDIKMTQSPSSIYASLGERVTITCKAS
QDIKSYLSWYQQKPRKSPKTLIYYATTLADGVPSRFSGSGSGQDFS
LTISSLESDDTATYYCLHHSESPWTFGGGTKLEIKRADAAPTVSIF
PPSSEQLTSGGASVVCFLNNFYPKDINVKWKIDGSERQNGVLNSWT
DQDSKDSTYSMSSTLTLTKDEYERHNSYTCEATHKTSTSPIVKSFN
RNEC

SEQ ID NO: 21 is the amino acid sequence of the heavy chain of IgG1 anti-CD33 antibody 5E10.7 (Ab_CD33_5E10.7_mmIgG1_Heavy Chain; MDT000552), which is specific for CD33^{FL} and CD33^{ΔE2}:

METDTLLLWVLLLWVPGSTGQVQLQQPGAEFVKPGASVKLSCKASG
YTFTSYWMQWVKQRPGQGLEWIGEIDSSDSYTNYNQKFKGMATLTV
DRSSSTAYMQLSSLTSEDSAVYYCARSRPYDWFPYWGQGTLVTVST
AKTTPPSVYPLAPGSAAQTNSMVTLGCLVKGYFPEPVTVTWNSGSL
SSGVHTFPAVLQSDLYTLSSSVTVPSSPRPSETVTCNVAHPASSTK
VDKKIVPRDCGCKPCICTVPEVSSVFIFPPKPKDVLTITLTPKVTC
VVVDISKDDPEVQFSWFVDDVEVHTAQTQPREEQFNSTFRSVSELP
IMHQDWLNGKEFKCRVNSAAFPAPIEKTISKTKGRPKAPQVYTIPP
PKEQMAKDKVSLTCMITDFFPEDITVEWQWNGQPAENYKNTQPIMN
TNGSYFVYSKLNVQKSNWEAGNTFTCSVLHEGLHNHHTEKSLSHSP
GK

SEQ ID NO: 22 is the amino acid sequence of the light chain of IgG1 anti-CD33 antibody 11D11 (Ab_CD33_11D11_mmIgG1_Light Chain; MDT000553), which is specific for CD33^{ΔE2}:

METDTLLLWVLLLWVPGSTGDIQMTQTTSSLSASLGDRVTISCRAS
QDISNYLNWYQQKPDGTVKLLIYYTSRLHSGVPSRFSGSGSGTDYS
LTISNLEQEDIATYFCQQGSTLPPTFGGGTKLEIKRADAAPTVSIF
PPSSEQLTSGGASVVCFLNNFYPKDINVKWKIDGSERQNGVLNSWT
DQDSKDSTYSMSSTLTLTKDEYERHNSYTCEATHKTSTSPIVKSFN
RNEC

SEQ ID NO: 23 is the amino acid sequence of the heavy chain of IgG1 anti-CD33 antibody 11D11 (Ab_CD33_11D11_mmIgG1_Heavy Chain; MDT000554), which is specific for CD33^{ΔE2}:

METDTLLLWVLLLWVPGSTGEVNLVESGGGLVQSGRSLRLSCATSG
FTFSDFYMEWVRQAPGKGLEWIAASRNKANDYTTEYKASVKGRFIV
SRDTSQSILYLQMNALRAEDTAIYYCTRDTGPMDYWGQGTSVTVSS
AKTTPPSVYPLAPGSAAQTNSMVTLGCLVKGYFPEPVTVTWNSGSL
SSGVHTFPAVLQSDLYTLSSSVTVPSSPRPSETVTCNVAHPASSTK
VDKKIVPRDCGCKPCICTVPEVSSVFIFPPKPKDVLTITLTPKVTC
VVVDISKDDPEVQFSWFVDDVEVHTAQTQPREEQFNSTFRSVSELP
IMHQDWLNGKEFKCRVNSAAFPAPIEKTISKTKGRPKAPQVYTIPP
PKEQMAKDKVSLTCMITDFFPEDITVEWQWNGQPAENYKNTQPIMN
TNGSYFVYSKLNVQKSNWEAGNTFTCSVLHEGLHNHHTEKSLSHSP
GK

SEQ ID NO: 24 is the amino acid sequence of the light chain of IgG1 anti-CD33 antibody 7E7 (Ab_CD33_7E7_mmIgG1_LC), which is specific for CD33^{ΔE2}:

METDTLLLWVLLLWVPGSTGDVVMTQTPLILSVTIGQPASISCKSS
QSLLDSDGKTYLSWLLQRPGQSPKRLIHLVSKLDSGVPDRFTGSGS
GTDFTLKISRVEAEDLGVYYCWQGTHFPLTFGAGTKLELKRADAAP
TVSIFPPSSEQLTSGGASVVCFLNNFYPKDINVKWKIDGSERQNGV
LNSWTDQDSKDSTYSMSSTLTLTKDEYERHNSYTCEATHKTSTSPI
VKSFNRNEC

SEQ ID NO: 25 is the amino acid sequence of the heavy chain of IgG1 anti-CD33 antibody 7E7 (Ab_CD33_7E7_mmIgG1_HC), which is specific for CD33^{ΔE2}:

METDTLLLWVLLLWVPGSTGQVTLKESGPGILQPSQTLSLTCSFSG
FSLNSYGMGIGWIRQPSGKGLEWLAHIWWDDNKYYKPDLKSRLTVS
KDTSKNQVFLKIANVDTTDTATYFCARDGGYSLFAYWGQGTLVTVS
VAKTTPPSVYPLAPGSAAQTNSMVTLGCLVKGYFPEPVTVTWNSGS
LSSGVHTFPAVLQSDLYTLSSSVTVPSSPRPSETVTCNVAHPASST
KVDKKIVPRDCGCKPCICTVPEVSSVFIFPPKPKDVLTITLTPKVT
CVVVDISKDDPEVQFSWFVDDVEVHTAQTQPREEQFNSTFRSVSEL
PIMHQDWLNGKEFKCRVNSAAFPAPIEKTISKTKGRPKAPQVYTIP
PPKEQMAKDKVSLTCMITDFFPEDITVEWQWNGQPAENYKNTQPIM
NTNGSYFVYSKLNVQKSNWEAGNTFTCSVLHEGLHNHHTEKSLSHS
PGK

SEQ ID NO: 26 is the amino acid sequence of human V-set Ig-like domain:

PNFWLQVQESVTVQEGLCVLVPCTFFHPIPYYDKNSPVHGYWFRE
GAIISRDSPVATNKLDQEVQEETQGRFRLLGDPSRNNCSLSIVDA
RRRDNGSYFFRMERG-STKYSYKSPQLS

SEQ ID NO: 27 is the amino acid sequence of human C2-set Ig-like domain:

PKILIPGTLEPGHSKNLTCSVSWACEQGTPPIFSWLSAAPTSLGP
RTTHSSVLIITPRPQDHGTNLTCQVKFAGAGVTTERTIQ

SEQ ID NOs: 28-31 are amino terminal peptides (neoepitopes) in CD33 confirmed by mass spectrophotometry (MS):

SEQ ID NO: 28:
MPLLLLLPLLWADLTHRPK
(E-value: 2E−3)
SEQ ID NO: 29:
M^{oxy}PLLLLLPLLWADLTHRPK
(E-value: 2E−3)
SEQ ID NO: 30:
LLLLLPLLWADLTHRPK
(E-value: 4.9E−4)
SEQ ID NO: 31:
PLLLLLPLLWADLTHRPK
(E-value: 1.2E−4)

SEQ ID NO: 32 is the amino acid sequence of human full-length CD33 (CD33^{FL}, signal peptide underline):

MPLLLLLPLLWAGALAMDPNFWLQVQESVTVQEGLCVLVPCTFFH
PIPYYDKNSPVHGYWFREGAIISRDSPVATNKLDQEVQEETQGRF
RLLGDPSRNNCSLSIVDARRRDNGSYFFRMERGSTKYSYKSPQLS
VHVTDLTHRPKILIPGTLEPGHSKNLTCSVSWACEQGTPPIFSWL
SAAPTSLGPRTTHSSVLIITPRPQDHGTNLTCQVKFAGAGVTTER
TIQLNVTYVPQNPTTGIFPGDGSGKQETRAGVVHGAIGGAGVTAL
LALCLCLIFFIVKTHRRKAARTAVGRNDTHPTTGSASPKHQKKSK
LHGPTETSSCSGAAPTVEMDEELHYASLNFHGMNPSKDTSTEYSE
VRTQ

SEQ ID NO: 33 is the amino acid sequence of the ΔE2 version of human CD33 (CD33^{ΔE2}) (signal peptide underlined):

MPLLLLLPLLWADLTHRPKILIPGTLEPGHSKNLTCSVSWACEQG
TPPIFSWLSAAPTSLGPRTTHSSVLIITPRPQDHGTNLTCQVKFA
GAGVTTERTIQLNVTYVPQNPTTGIFPGDGSGKQETRAGVVHGAI
GGAGVTALLALCLCLIFFIVKTHRRKAARTAVGRNDTHPTTGSAS
PKHQKKSKLHGPTETSSCSGAAPTVEMDEELHYASLNFHGMNPSK
DTSTEYSEVRTQ

SEQ ID NO: 34 is the amino acid sequence of CD33^{ΔE7a} which lacks most of the intracellular domain of human CD33 (C-terminal truncation) (signal peptide underlined):

MPLLLLLPLLWAGALAMDPNFWLQVQESVTVQEGLCVLVPCTFFH
PIPYYDKNSPVHGYWFREGAIISRDSPVATNKLDQEVQEETQGRF
RLLGDPSRNNCSLSIVDARRRDNGSYFFRMERGSTKYSYKSPQLS
VHVTDLTHRPKILIPGTLEPGHSKNLTCSVSWACEQGTPPIFSWL
SAAPTSLGPRTTHSSVLIITPRPQDHGTNLTCQVKFAGAGVTTER
TIQLNVTYVPQNPTTGIFPGDGSGKQETRAGVVHGAIGGAGVTAL
LALCLCLIFFIVKTHRRKAARTAVGRNDTHPTTGSASPVR

SEQ ID NO: 35 is the amino acid sequence of cynomolgus CD33 (MfCD33_G7PYHO) shown in the alignment in FIG. 2:

MPLLLLPLLWAGALAMDPRVRLEVQESVTVQEGLCVLVPCTFFHP
VPYHTRNSPVHGYWFREGAIVSLDSPVATNKLDQEVQEETQGRFR
LLGDPSRNNCSLSIVDARRRDNGSYFFRMEKGSTKYSYKSTQLSV
HVTDLTHRPQILIPGALDPDHSKNLTCSVPWACEQGTPPIFSWMS
AAPTSLGLRTTHSSVLIITPRPQDHGTNLTCQVKFPGAGVTTERT
IQLNVSYASQNPRTDIFLGDGSGRKARKQGVVQGAIGGAGVTVLL
ALCLCLIFFTVQ

SEQ ID NO: 36 is the amino acid sequence of human CD33 (HsCD33_P20138) shown in the alignment in FIG. 2:

MPLLLLLPLLWAGALAMDPNFWLQVQESVTVQEGLCVLVPCTFFH
PIPYYDKNSPVHGYWFREGAIISRDSPVATNKLDQEVQEETQGRF
RLLGDPSRNNCSLSIVDARRRDNGSYFFRMERGSTKYSYKSPQLS
VHVTDLTHRPKILIPGTLEPGHSKNLTCSVSWACEQGTPPIFSWL
SAAPTSLGPRTTHSSVLIITPRPQDHGTNLTCQVKFAGAGVTTER
TIQLNVTYVPQNPTTGIFPGDGSGKQETRAGVVHGAIGGAGVTAL
LALCLCLIFFIVQ

SEQ ID NO: 37 is the amino acid sequence of murine CD33 (MmCD33_Q63994) shown in the alignment in FIG. 2:

MLWPLPLFLLCAGSLAQDLEFQLVAPESVTVEEGLCVHVPCSVFY
PSIKLTLGPVTGSWLRKGVSLHEDSPVATSDPRQLVQKATQGRFQ
LLGDPQKHDCSLFIRDAQKNDTGMYFFRVVREPFVRYSYKKSQLS
LHVTSLSRTPDIIIPGTLEAGYPSNLTCSVPWACEQGTPPTFSWM
STALTSLSSRTTDSSVLTFTPQPQDHGTKLTCLVTFSGAGVTVER
TIQLNVTRKSGQMRELVLVAVG-EATVKLLILGLCLVFLIVMF

From the anti-CD3 antibody OKT3:

CDRL1:
(SEQ ID NO: 38)
SASSSVSYMN
CDRL2:
(SEQ ID NO: 39)
RWIYDTSKLAS
CDRL3:
(SEQ ID NO: 40)
QQWSSNPFT
CDRH1:
(SEQ ID NO: 41)
KASGYTFTRYTMH
CDRH2:
(SEQ ID NO: 42)
INPSRGYTNYNQKFKD
CDRH3:
(SEQ ID NO: 43)
YYDDHYCLDY

SEQ ID NO: 44 is the amino acid sequence of a scFv derived from OKT3 which retains the capacity to bind CD3:

QVQLQQSGAELARPGASVKMSCKASGYTFTRYTMHWVKQRPGQGL
EWIGYINPSRGYTNYNQKFKDKATLTTDKSSSTAYMQLSSLTSED
SAVYYCARYYDDHYCLDYWGQGTTLTVSSSGGGGSGGGGSGGGGS
QIVLTQSPAIMSASPGEKVTMTCSASSSVSYMNWYQQKSGTSPKR
WIYDTSKLASGVPAHFRGSGSGTSYSLTISGMEAEDAATYYCQQW
SSNPFTFGSGTKLEINR

From the anti-CDS antibody 20G6-F3

CDRL1:
(SEQ ID NO: 45)
QSLVHNNGNTY
DRL2:
(SEQ ID NO: 46)
KVS
DRL3:
(SEQ ID NO: 47)
GQGTQYPFT
DRH1:
(SEQ ID NO: 48)
GFTFTKAW
DRH2:
(SEQ ID NO: 49)
IKDKSNSYAT
DRH3:
(SEQ ID NO: 50)
RGVYYALSPFDY

From the anti-CDS antibody 484-D7

CDRL1:
(SEQ ID NO: 51)
QSLVHDNGNTY
DRL2:
(SEQ ID NO: 52)
KVS
DRL3:
(SEQ ID NO: 53)
GQGTQYPFT
DRH1:
(SEQ ID NO: 54)
GFTFSNAW
DRH2:
(SEQ ID NO: 55)
IKARSNNYAT
DRH3:
(SEQ ID NO: 56)
RGTYYASKPFDY

From the anti-CDS antibody 4E7-C9

CDRL1:
(SEQ ID NO: 57)
QSLEHNNGNTY
DRL2:
(SEQ ID NO: 58)
KVS
DRL3:
(SEQ ID NO: 59)
GQGTQYPFT
DRH1:
(SEQ ID NO: 60)
GFTFSNAW
DRH2:
(SEQ ID NO: 61)
IKDKSNNYAT
DRH3:
(SEQ ID NO: 62)
RYVHYGIGYAMDA

From the anti-CD3 18F5-H10 antibody:

CDRL1:
(SEQ ID NO: 63)
QSLVHTNGNTY
DRL2:
(SEQ ID NO: 64)
KVS
DRL3:
(SEQ ID NO: 65)
GQGTHYPFT
DRH1:
(SEQ ID NO: 66)
GFTFTNAW
DRH2:
(SEQ ID NO: 67)
KDKSNNYAT
DRH3:
(SEQ ID NO: 68)
RYVHYRFAYALDA

From the anti-CD8 antibody OKT8:

CDRL1:
(SEQ ID NO: 69)
RTSRSISQYLA
CDRL2:
(SEQ ID NO: 70)
SGSTLQS
CDRL3:
(SEQ ID NO: 71)
QQHNENPLT
CDRH1:
(SEQ ID NO: 72)
GFNIKD
CDRH2:
(SEQ ID NO: 73)
RIDPANDNT
CDRH3:
(SEQ ID NO: 74)
GYGYYVFDH

SEQ ID NO: 75 is the amino acid sequence of a variable light chain region of the sequence of a binding domain that binds and blocks the NK cell inhibitory receptors KIR2DL1 and KIR2DL2/3:

EIVLTQSPVTLSLSPGERATLSCRASQSVSSYLAWYQQKPGQAPRLLIYD
ASNRATGIPARFSGSGSGTDFTLTISSLEPEDFAVYYCQQRSNWMYTFGQ
GTKLEIKRT

SEQ ID NO: 76 is the amino acid sequence of a variable heavy chain region of the sequence of a binding domain that binds and blocks the NK cell inhibitory receptors KIR2DL1 and KIR2DL2/3:

QVQLVQSGAEVKKPGSSVKVSCKASGGTFSFYAISWVRQAPGQGLEWMGG
FIPIFGAANYAQKFQGRVTITADESTSTAYMELSSLRSDDTAVYYCARIP
SGSYYYDYDMDVWGQGTTVTVSS

SEQ ID NOs: 77-82 are linkers:

(SEQ ID NO: 77)
GGSGGGSGGSG
(SEQ ID NO: 78)
GGSGGGSGSG
(SEQ ID NO: 79)
GGSGGGSG
(SEQ ID NO: 80)
GGGGSGGGGS
(SEQ ID NO: 81)
GGGSGGGS
(SEQ ID NO: 82)
GGSGGS.

SEQ ID NO: 83 is the amino acid sequence of the heavy chain of anti-CD33 antibody 1H7 (Ab_CD33_1H7_HC):

METDTLLLWVLLLWVPGSTGQVQLQQSGAELVKPGASVKISCKASGYAFS
NYWMNWVKQRPGKGLEWIGQINPGDGDTNYNGKFKGKATLTADKSSSTAY
MQLSSLTSEDSAVYFCAREDRDYFDYWGQGTTLTVSSAKTTAPSVYPLAP
VCGDTTGSSVTLGCLVKGYFPEPVTLTWNSGSLSSGVHTFPAVLQSDLYT
LSSSVTVTSSTWPSQSITCNVAHPASSTKVDKKIEPRGPTIKPCPPCKCP
APNLLGGPSVFIFPPKIKDVLMISLSPIVTCVVVDVSEDDPDVQISWFVN
NVEVHTAQTQTHREDYNSTLRVVSALPIQHQDWMSGKEFKCKVNNKDLPA
PIERTISKPKGSVRAPQVYVLPPPEEEMTKKQVTLTCMVTDFMPEDIYVE
WTNNGKTELNYKNTEPVLDSDGSYFMYSKLRVEKKNWVERNSYSCSVVHE
GLHNHHTTKSFSRTPGK

SEQ ID NO: 84 is the amino acid sequence of the light chain of anti-CD33 antibody 1H7(Ab_CD33_1H7_LC)

METDTLLLWVLLLWVPGSTGDIQMTQTTSSLSASLGDRVTISCRASQDIN
YYLNWYQQKPDGTVKLLIYYSSRLHSGVPSRFSGSGSGTDFSLTISNLEQ
EDIATYFCQQDDALPYTFGGGTKLEIKRADAAPTVSIFPPSSEQLTSGGA
SVVCFLNNFYPKDINVKWKIDGSERQNGVLNSWTDQDSKDSTYSMSSTLT
LTKDEYERHNSYTCEATHKTSTSPIVKSFNRNEC

In anti-CD33 antibody 1H7:

CDRL1:
(SEQ ID NO: 85)
RASQDINYYLN
CDRL2:
(SEQ ID NO: 86)
YSSRLHSG
CDRL3:
(SEQ ID NO: 87)
QQDDALPYT
CDRH1:
(SEQ ID NO: 88)
GYAFSNYWMN
CDRH2:
(SEQ ID NO: 89)
QINPGDGDTNYNG
CDRH3:
(SEQ ID NO: 90)
EDRDYFDY

SEQ ID NO: 91 is the amino acid sequence of a 1H7-CD3 bispecific T-cell engaging antibody:

METDTLLLWVLLLWVPGSTGDIQMTQTTSSLSASLGDRVTISCRASQDIN
YYLNWYQQKPDGTVKLLIYYSSRLHSGVPSRFSGSGSGTDFSLTISNLEQ
EDIATYFCQQDDALPYTFGGGTKLEIKGGGGSGGGGSGGGGSQVQLQQSG
AELVKPGASVKISCKASGYAFSNYWMNWVKQRPGKGLEWIGQINPGDGDT
NYNGKFKGKATLTADKSSSTAYMQLSSLTSEDSAVYFCAREDRDYFDYWG
QGTTLTVSSGGGGSEVQLVESGGGLVQPGGSLKLSCAASGFTFNKYAMNW
VRQAPGKGLEWVARIRSKYNNYATYYADSVKDRFTISRDDSKNTAYLQMN
NLKTEDTAVYYCVRHGNFGNSYISYWAYWGQGTLVTVSSGGGGSGGGGSG
GGGSQTVVTQEPSLTVSPGGTVTLTCGSSTGAVTSGNYPNWVQQKPGQAP
RGLIGGTKFLAPGTPARFSGSLLGGKAALTLSGVQPEDEAEYYCVLW
YSNRWVFGGGTKLTVLHHHHHH

SEQ ID NO: 92 is the amino acid sequence of a light chain 1H7-CD3 engager:

METDTLLLWVLLLWVPGSTGDIQMTQTTSSLSASLGDRVTISCRASQDIN
YYLNWYQQKPDGTVKLLIYYSSRLHSGVPSRFSGSGSGTDFSLTISNLEQ
EDIATYFCQQDDALPYTFGGGTKLEIKRADAAPTVSIFPPSSEQLTSGGA
SVVCFLNNFYPKDINVKWKIDGSERQNGVLNSWTDQDSKDSTYSMSSTLT
LTKDEYERHNSYTCEATHKTSTSPIVKSFNRNECGGGGSGGGGSGGGGSE
VQLVESGGGLVQPGGSLKLSCAASGFTFNKYAMNWVRQAPGKGLEWVARI
RSKYNNYATYYADSVKDRFTISRDDSKNTAYLQMNNLKTEDTAVYYCVRH
GNFGNSYISYWAYWGQGTLVTVSSGGGGSGGGGSGGGGSQTVVTQEPSLT
VSPGGTVTLTCGSSTGAVTSGNYPNWVQQKPGQAPRGLIGGTKFLAPGTP
ARFSGSLLGGKAALTLSGVQPEDEAEYYCVLWYSNRWVFGGGTKLTVLHH
HHHH

SEQ ID NO: 93 is the amino acid sequence of a heavy chain 1H7-CD3 engager:

METDTLLLWVLLLWVPGSTGQVQLQQSGAELVKPGASVKISCKASGYAFS
NYWMNWVKQRPGKGLEWIGQINPGDGDTNYNGKFKGKATLTADKSSSTAY
MQLSSLTSEDSAVYFCAREDRDYFDYWGQGTTLTVSSAKTTAPSVYPLAP
VCGDTTGSSVTLGCLVKGYFPEPVTLTWNSGSLSSGVHTFPAVLQSDLYT
LSSSVTVTSSTWPSQSITCNVAHPASSTKVDKKIEPRGPTIKPCPPCKCP
APNLLGGPSVFIFPPKIKDVLMISLSPIVTCVVVDVSEDDPDVQISWFVN
NVEVHTAQTQTHREDYNSTLRVVSALPIQHQDWMSGKEFKCKVNNKDLPA
PIERTISKPKGSVRAPQVYVLPPPEEEMTKKQVTLTCMVTDFMPEDIYVE
WTNNGKTELNYKNTEPVLDSDGSYFMYSKLRVEKKNWVERNSYSCSVVHE
GLHNHHTTKSFSRTPGK

SEQ ID NOs: 94-153 are CDR sequences from anti-CD33 antibodies, as follows:

| SEQ | | Antibody |
|---|---|---|
| ID | | and |
| NO: | Sequence | CDR |
| 94 | GYAFSNYWMN | 5D12_CDRH1 |
| 95 | QIYPGNFNTDYNGQFKG | 5D12_CDRH2 |
| 96 | FFDFGAYFTLDY | 5D12_CDRH3 |
| 97 | KASQDIKSYLS | 5D12_CDRL1 |
| 98 | YATTLAD | 5D12_CDRL2 |
| 99 | LHHGESPWT | 5D12_CDRL3 |
| 100 | SGFTFSDFYMY | 8F5_CDRH1 |
| 101 | FISNAGVTTYYPDTVEG | 8F5_CDRH2 |
| 102 | SDYDGAWFPY | 8F5_CDRH3 |
| 103 | KSSQSLLYSRNQYNFLA | 8F5_CDRL1 |
| 104 | WASTRES | 8F5_CDRL2 |
| 105 | QQYYSYPYT | 8F5_CDRL3 |
| 106 | GYTFTTYWMH | 12B12_CDRH1 |
| 107 | AIYPGNSDTSYNQ | 12B12_CDRH2 |
| 108 | YDGYHFI | 12B12_CDRH3 |
| 109 | RSSQSLLHSNGITYLY | 12B12_CDRL1 |
| 110 | QMSNLAS | 12B12_CDRL2 |
| 111 | AQNLELPPT | 12B12_CDRL3 |
| 112 | GYAFTNYLIE | 4H10_CDRH1 |
| 113 | VIHPGNNSTSYNA | 4H10_CDRH2 |
| 114 | YGYDERNAMDY | 4H10_CDRH3 |
| 115 | QSLLYSNGKTYLH | 4H10_CDRL1 |
| 116 | VPDRFTGSGSGTDFTL | 4H10_CDRL2 |
| 117 | GTHFPRTFG | 4H10_CDRL3 |
| 118 | GYTFTSYWMH | 11D5_CDRH1 |
| 119 | AIYCGNSDTSYNQ | 11D5_CDRH2 |
| 120 | YDGYHFDY | 11D5_CDRH3 |
| 121 | RSNKSLLHSNGITYLY | 11D5_CDRL1 |
| 122 | QMSNLAS | 11D5_CDRL2 |
| 123 | AQNLELPPT | 11D5_CDRL3 |
| 124 | GYTFTDYTLH | 13E11_CDRH1 |
| 125 | WFYPTSGSINYNE | 13E11_CDRH2 |
| 126 | HKFGFDY | 13E11_CDRH3 |
| 127 | KASHGVEYAGAHYMN | 13E11_CDRL1 |
| 128 | AASNLGS | 13E11_CDRL2 |
| 129 | QQSNEDPRT | 13E11_CDRL3 |
| 136 | SGFTFSDFYME | 11D11_CDRH1 |
| 137 | ASRNKANDYTTEY | 11D11_CDRH2 |
| 138 | DTGPMDY | 11D11_CDRH3 |
| 139 | RASQDISNYLN | 11D11_CDRL1 |
| 140 | YTSRLHS | 11D11_CDRL2 |
| 141 | QQGSTLPPT | 11D11_CDRL3 |
| 142 | GFSLNSYGMGIG | 7E7_CDRH1 |
| 143 | HIWWDDNKYYKPDLKS | 7E7_CDRH2 |
| 144 | DGGYSLFAY | 7E7_CDRH3 |
| 145 | KSSQSLLDSDGKTYLS | 7E7_CDRL1 |
| 146 | VSKLDSG | 7E7_CDRL2 |
| 147 | WQGTHFPLT | 7E7_CDRL3 |
| 130 | GYTFTSYWMQ | 5E10.7_CDRH1 |
| 131 | EIDSSDSYTNYNQ | 5E10.7_CDRH2 |
| 132 | SRPYDWFPY | 5E10.7_CDRH3 |
| 133 | KASQDIKSYLS | 5E10.7_CDRL1 |
| 134 | YATTLAD | 5E10.7_CDRL2 |
| 135 | LHHSESPWT | 5E10.7_CDRL3 |
| 148 | GYTFTDYDMH | 2D5_CDRH1 |
| 149 | AIDPETGGTAYNQNF | 2D5_CDRH2 |
| 150 | DYDYFGV | 2D5_CDRH3 |
| 151 | KASQNVGTNVV | 2D5_CDRL1 |
| 152 | SASDRYS | 2D5_CDRL2 |
| 153 | QQYNIYPYT | 2D5_CDRL3 |

SEQ ID NO: 154 is the amino acid sequence of the light chain of anti-CD33 antibody 2D5:

MESQTQVFVYMLLWLSGVDGDIVMTQSQKFMSTSVGDRVSVTCKASQNVG
TNVVWYHKKPGQSPKGLIYSASDRYSGVPDRFTGSGSGTDFTLTINNVQS
EDLAEYFCQQYNIYPYTFGGGTKLEIKR

SEQ ID NO: 155 is the amino acid sequence of the heavy chain of anti-CD33 antibody 2D5:

MEWSWVCLFLLSVIAGVQSQVQLQQSGAELVRPGASVTLSCKASGYTFTD
YDMHWVKQTPVHGLEWIGAIDPETGGTAYNQNFKGKAILTVDKSSRIAYM
ELRSLTSEDSAVFYCTSDYDYFGVWGTGTTVTVSS

SEQ ID NO: 156 is the amino acid sequence of CD33^{FL} signal peptide fused to the C-terminal end of CD33^{ΔE2} , from FIG. 14A:

MPLLLLLPLLWAGALAMDLTHRPKILIPGTLEPGHSKNLTCSVS

SEQ ID NO: 157 is the amino acid sequence of CD33^{ΔE2} signal peptide fused to the C-terminal end of CD33 FL, from FIG. 14A:

MPLLLLLPLLWADLTHRPKILIPNFWLQVQESVTVQEGLCVLVPCTFF

What is claimed is:

1. A CD33-binding isolated monoclonal antibody or CD33-binding fragment thereof comprising a variable light chain comprising a complementarity determining region light (CDRL) 1, a CDRL2, and a CDRL3 and a variable heavy chain comprising a CDR heavy (H) 1, a CDRH2, and a CDRH3, wherein
the CDRL1 has a sequence as set forth in SEQ ID NO: 85, the CDRL2 has a sequence as set forth in SEQ ID NO: 86, the CDRL3 has a sequence as set forth in SEQ ID NO: 87, the CDRH1 has a sequence as set forth in SEQ ID NO: 88, the CDRH2 has a sequence as set forth in SEQ ID NO: 89, and the CDRH3 has a sequence as set forth in SEQ ID NO: 90;
the CDRL1 has a sequence as set forth in SEQ ID NO: 151, the CDRL2 has a sequence as set forth in SEQ ID NO: 152, the CDRL3 has a sequence as set forth in SEQ ID NO: 153, the CDRH1 has a sequence as set forth in SEQ ID NO: 148, the CDRH2 has a sequence as set forth in SEQ ID NO: 149, and the CDRH3 has a sequence as set forth in SEQ ID NO: 150
the CDRL1 has a sequence as set forth in SEQ ID NO: 109, the CDRL2 has a sequence as set forth in SEQ ID NO: 110, the CDRL3 has a sequence as set forth in SEQ ID NO: 111, the CDRH1 has a sequence as set forth in SEQ ID NO: 106, the CDRH2 has a sequence as set forth in SEQ ID NO: 107, and the CDRH3 has a sequence as set forth in SEQ ID NO: 108;
the CDRL1 has a sequence as set forth in SEQ ID NO: 115, the CDRL2 has a sequence as set forth in SEQ ID NO: 116, the CDRL3 has a sequence as set forth in SEQ ID NO: 117, the CDRH1 has a sequence as set forth in SEQ ID NO: 112, the CDRH2 has a sequence as set forth in SEQ ID NO: 113, and the CDRH3 has a sequence as set forth in SEQ ID NO: 114;
the CDRL1 has a sequence as set forth in SEQ ID NO: 121, the CDRL2 has a sequence as set forth in SEQ ID NO: 122, the CDRL3 has a sequence as set forth in SEQ ID NO: 123, the CDRH1 has a sequence as set forth in SEQ ID NO: 118, the CDRH2 has a sequence as set forth in SEQ ID NO: 119, and the CDRH3 has a sequence as set forth in SEQ ID NO: 120;
the CDRL1 has a sequence as set forth in SEQ ID NO: 127, the CDRL2 has a sequence as set forth in SEQ ID NO: 128, the CDRL3 has a sequence as set forth in SEQ ID NO: 129, the CDRH1 has a sequence as set forth in SEQ ID NO: 124, the CDRH2 has a sequence as set forth in SEQ ID NO: 125, and the CDRH3 has a sequence as set forth in SEQ ID NO: 126;
the CDRL1 has a sequence as set forth in SEQ ID NO: 139, the CDRL2 has a sequence as set forth in SEQ ID NO: 140, the CDRL3 has a sequence as set forth in SEQ ID NO: 141, the CDRH1 has a sequence as set forth in SEQ ID NO: 136, the CDRH2 has a sequence as set forth in SEQ ID NO: 137, and the CDRH3 has a sequence as set forth in SEQ ID NO: 138;
the CDRL1 has a sequence as set forth in SEQ ID NO: 145, the CDRL2 has a sequence as set forth in SEQ ID NO: 146, the CDRL3 has a sequence as set forth in SEQ ID NO: 147, the CDRH1 has a sequence as set forth in SEQ ID NO: 142, the CDRH2 has a sequence as set forth in SEQ ID NO: 143, and the CDRH3 has a sequence as set forth in SEQ ID NO: 144;
the CDRL1 has a sequence as set forth in SEQ ID NO: 97, the CDRL2 has a sequence as set forth in SEQ ID NO: 98, the CDRL3 has a sequence as set forth in SEQ ID NO: 99, the CDRH1 has a sequence as set forth in SEQ ID NO: 94, the CDRH2 has a sequence as set forth in SEQ ID NO: 95, and the CDRH3 has a sequence as set forth in SEQ ID NO: 96; or
the CDRL1 has a sequence as set forth in SEQ ID NO: 103, the CDRL2 has a sequence as set forth in SEQ ID NO: 104, the CDRL3 has a sequence as set forth in SEQ ID NO: 105, the CDRH1 has a sequence as set forth in SEQ ID NO: 100, the CDRH2 has a sequence as set forth in SEQ ID NO: 101, and the CDRH3 has a sequence as set forth in SEQ ID NO: 102.

2. The CD33-binding isolated monoclonal antibody or CD33-binding fragment thereof of claim 1, wherein
the variable light chain has the sequence as set forth in SEQ ID NO: 84 or a sequence having at least 90% sequence identity thereto and the variable heavy chain has the sequence as set forth in SEQ ID NO: 83 or a sequence having at least 90% sequence identity thereto;
the variable light chain has the sequence as set forth in SEQ ID NO: 154 or a sequence having at least 90% sequence identity thereto and the variable heavy chain has the sequence as set forth in SEQ ID NO: 155 or a sequence having at least 90% sequence identity thereto;
the variable light chain has the sequence as set forth in SEQ ID NO: 12 or a sequence having at least 90% sequence identity thereto and the variable heavy chain has the sequence as set forth in SEQ ID NO: 13 or a sequence having at least 90% sequence identity thereto;
the variable light chain has the sequence as set forth in SEQ ID NO: 14 or a sequence having at least 90% sequence identity thereto and the variable heavy chain has the sequence as set forth in SEQ ID NO: 15 or a sequence having at least 90% sequence identity thereto;
the variable light chain has the sequence as set forth in SEQ ID NO: 16 or a sequence having at least 90% sequence identity thereto and the variable heavy chain has the sequence as set forth in SEQ ID NO: 17 or a sequence having at least 90% sequence identity thereto;
the variable light chain has the sequence as set forth in SEQ ID NO: 18 or a sequence having at least 90% sequence identity thereto and the variable heavy chain has the sequence as set forth in SEQ ID NO: 19 or a sequence having at least 90% sequence identity thereto;
the variable light chain has the sequence as set forth in SEQ ID NO: 22 or a sequence having at least 90% sequence identity thereto and the variable heavy chain has the sequence as set forth in SEQ ID NO: 23 or a sequence having at least 90% sequence identity thereto;
the variable light chain has the sequence as set forth in SEQ ID NO: 24 or a sequence having at least 90% sequence identity thereto and the variable heavy chain has the sequence as set forth in SEQ ID NO: 25 or a sequence having at least 90% sequence identity thereto;
the variable light chain has the sequence as set forth in SEQ ID NO: 8 or a sequence having at least 90% sequence identity thereto and the variable heavy chain has the sequence as set forth in SEQ ID NO: 9 or a sequence having at least 90% sequence identity thereto;
the variable light chain has the sequence as set forth in SEQ ID NO: 10 or a sequence having at least 90% sequence identity thereto and the variable heavy chain has the sequence as set forth in SEQ ID NO: 11 or a sequence having at least 90% sequence identity thereto.

3. The CD33-binding isolated monoclonal antibody or CD33-binding fragment thereof of claim 1, in a form of a whole antibody, a single chain variable fragment (scFv), or an immunoconjugate.

4. The CD33-binding isolated monoclonal antibody or CD33-binding fragment thereof of claim 3, wherein the immunoconjugate comprises the antibody or binding fragment thereof conjugated to a toxin, radioactive isotope, or a drug.

5. The CD33-binding isolated monoclonal antibody or CD33-binding fragment thereof of claim 4, wherein the radioactive isotope comprises iodine-131, indium-111, yttrium-90, lutetium-177, astatine-211, bismuth-212 or bismuth-213.

6. The CD33-binding isolated monoclonal antibody or CD33-binding fragment thereof of claim 1, wherein the CD33-binding isolated monoclonal antibody or CD33-binding fragment thereof is covalently conjugated to polyethylene glycol (PEG).

7. The CD33-binding isolated monoclonal antibody or CD33-binding fragment thereof of claim 1, wherein the CD33-binding isolated monoclonal antibody or CD33-binding fragment thereof is part of an antibody-drug conjugate comprising maytansinoid, dolastatin, auristatin, calicheamicin, pyrrolobenzodiazepine (PBD), nemorubicin, anthracycline, duocarmycin, vinca alkaloid, taxane, trichothecene, camptothecin, or elinafide.

8. The CD33-binding isolated monoclonal antibody or CD33-binding fragment thereof of claim 1, wherein the antibody or binding fragment thereof further comprises a human heavy chain constant region and/or a human light chain constant region.

9. The CD33-binding isolated monoclonal antibody or CD33-binding fragment thereof of claim 8, wherein the human heavy chain constant region comprises an IgG, IgM, IgD, IgA, or IgE heavy chain constant region.

10. The CD33-binding isolated monoclonal antibody or CD33-binding fragment thereof of claim 8, wherein the human light chain constant region comprises a human kappa (lgk) light chain constant region or a human lambda (lgA) light chain constant region.

11. The CD33-binding isolated monoclonal antibody or CD33-binding fragment thereof of claim 1, wherein the antibody or binding fragment thereof is humanized.

12. A bispecific antibody molecule or bispecific antigen binding fragment thereof comprising the variable light chain and the variable heavy chain of the CD33-binding monoclonal antibody or antigen binding fragment thereof of claim 1.

13. The bispecific antibody molecule or bispecific antigen binding fragment thereof of claim 12, wherein the bispecific antibody molecule or bispecific antigen binding fragment thereof further comprises a CD3 binding domain or a CD8 binding domain.

14. The bispecific antibody molecule or bispecific antigen binding fragment thereof of claim 13, wherein the CD3 binding domain comprises
a variable light chain comprising
a CDRL1 having the sequence as set forth in SEQ ID NO: 38,
a CDRL2 having the sequence as set forth in SEQ ID NO: 39, and
a CDRL3 having the sequence as set forth in SEQ ID NO: 40, and
a variable heavy chain comprising
a CDRH1 having the sequence as set forth in SEQ ID NO: 41,
a CDRH2 having the sequence as set forth in SEQ ID NO: 42, and
a CDRH3 having the sequence as set forth in SEQ ID NO: 43.

15. The bispecific antibody molecule or bispecific antigen binding fragment thereof of claim 13, wherein the CD8 binding domain comprises
a variable light chain comprising
a CDRL1 having the sequence as set forth in SEQ ID NO: 69,
a CDRL2 having the sequence as set forth in SEQ ID NO: 70, and
a CDRL3 having the sequence as set forth in SEQ ID NO: 71, and
a variable heavy chain comprising
a CDRH1 having the sequence as set forth in SEQ ID NO: 72,
a CDRH2 having the sequence as set forth in SEQ ID NO: 73, and
a CDRH3 having the sequence as set forth in SEQ ID NO: 74.

16. A chimeric antigen receptor (CAR) comprising
an extracellular component comprising a CD33-binding isolated monoclonal antibody or CD33-binding fragment thereof of claim 1,
a transmembrane domain, and
an effector domain,
wherein the transmembrane domain links the extracellular component to the effector domain.

17. The CAR of claim 16, wherein the effector domain comprises one or more of: 4-1BB, CD3ε, CD3δ, CD3ζ, CD27, CD28, CD79A, CD79B, CARD11, DAP10, FcRα, FcRβ, FcRγ, Fyn, HVEM, ICOS, Lck, LAG3, LAT, LRP, NOTCH1, Wnt, NKG2D, OX40, ROR2, Ryk, SLAMF1, Slp76, pTα, TCRα, TCRβ, TRIM, Zap70, or PTCH2.

18. The CAR of claim 16, wherein the transmembrane domain comprises at least a transmembrane region(s) of) an alpha, beta, or zeta chain of a T-cell receptor, CD28, CD3, CD45, CD4, CD8, CD9, CD16, CD22, CD33, CD37, CD64, CD80, CD86, CD134, CD137, or CD154.

19. The CAR of claim 16, further comprising a spacer region.

20. A composition comprising the CD33-binding isolated monoclonal antibody or CD33-binding fragment thereof of claim 1 and a pharmaceutically acceptable carrier.
